(19) United States
(12) Patent Application Publication
(10) Pub. No.: US 2023/0121116 A1
(43) Pub. Date: Apr. 20, 2023

(54) USE OF QUINAZOLINE-BASED TYROSINE KINASE INHIBITORS FOR THE TREATMENT OF CANCERS WITH NRG1 FUSIONS

(71) Applicant: Board of Regents, The University of Texas System, Austin, TX (US)

(72) Inventors: John HEYMACH, Houston, TX (US); Jacqulyne ROBICHAUX, Houston, TX (US)

(73) Assignee: Board of Regents, The University of Texas System, Austin, TX (US)

(21) Appl. No.: 17/759,817

(22) PCT Filed: Jan. 29, 2021
(86) PCT No.: PCT/US21/15704

§ 371 Date: Jul. 29, 2022

Related U.S. Application Data
(60) Provisional application No. 62/967,282, filed on Jan. 29, 2020.

(51) Int. Cl. A61K 31/519; A61K 45/06; A61P 35/00

(52) U.S. Cl. CPC A61K 31/519; A61K 45/06; A61P 35/00

(57) ABSTRACT

Provided herein are methods of selecting cancer patients for treatment with quinazoline-based tyrosine kinase inhibitors, either alone or in combination with anti-HER2/HER3 antibodies, as well as methods of treating cancer patients so selected. Cancer patients are selected for treatment if their cancer has an NRG1 fusion. Selected patients are then treated with quinazoline-based tyrosine kinase inhibitors alone or in combination with anti-HER2/HER3 antibodies.

### REFERENCE TO RELATED APPLICATIONS

The present application claims the priority benefit of U.S. provisional application No. 62/967,282, filed Jan. 29, 2020, the entire contents of which is incorporated herein by reference.

### BACKGROUND

### 1. Field

The present invention relates generally to the fields of medicine and oncology. More particularly, it concerns methods for selecting cancer patients for treatment with quinazoline-based tyrosine kinase inhibitors (TKIs), or with a combination of quinazoline-based TKIs and anti-HER2/HER3 antibodies, as well as methods of treating cancer patients so selected.

### 2. Description of Related Art

NRG1 fusions occur in 0.3% of non-small cell lung cancer (NSCLC), and have been observed in several other cancer types including gallbladder (0.5%), breast (0.2%), ovarian (0.4%), and colorectal (0.1%) cancers (Jonna et al., 2019). Common NRG1 fusions partners are CD74 (29% of NRG1 fusions), ATP1B1 (10% of NRG1 fusions), and SDC4 (7% of NRG1 fusions) (Jonna et al., 2019). NRG1 binds the HER3 receptor to cause preferential hetero-dimerization with HER2 (Shin et al., 2018; Jung et al., 2015; Fernandez-Cuesta et al., 2014), one of the most potent forms of ERBB family signaling (Holbro et al., 2003). Previous reports have shown that targeting the HER2/HER3 signaling pathway with single agents can be effective in inhibiting NRG1-fusion driven ErbB signaling (Shin et al., 2018; Fernandez-Cuesta et al., 2014; Drilon et al., 2018). However, there are no approved targeted therapies for patients with NRG1 fusions.

### SUMMARY

In one embodiment, provided herein are methods of treating a patient having a cancer, the methods comprising (a) determining or having determined whether the patient's cancer has an NRG1 fusion; (b) selecting or having selected the patient for treatment with a quinazoline-based tyrosine kinase inhibitor (TKI) when the patient's cancer has an NRG1 fusion; and (c) administering or having administered to the selected patient a therapeutically effective amount of the quinazoline-based TKJ. In some aspects, step (a) comprises (i) obtaining or having obtained a biological sample from the patient; and (ii) performing or having performed an assay on the biological sample to determine the patient's cancer has an NRG1 fusion.

In one embodiment, provided herein are methods of treating a patient having a cancer, the methods comprising administering to the patient a therapeutically effective amount of a quinazoline-based TKI, wherein the cancer has an NRG1 fusion. In one embodiment, provided herein are compositions comprising a therapeutically effective amount of a quinazoline-based TKI, for use in the treatment of cancer in a patient, wherein the patient's cancer has an NRG1 fusion.

In one embodiment, provided herein are methods of selecting a patient having a cancer for treatment with a quinazoline-based TKI, the methods comprising (a) determining or having determined whether the patient's cancer has an NRG1 fusion; (b) selecting or having selected the patient for treatment with a quinazoline-based TKI when the patient's cancer has an NRG1 fusion. In some aspects, step (a) comprises (i) obtaining or having obtained a biological sample from the patient; and (ii) performing or having performed an assay on the biological sample to determine the patient's cancer has an NRG1 fusion. In some aspects, the methods further comprise (c) administering or having administered to the selected patient a therapeutically effective amount of a quinazoline-based TKI.

In one embodiment, provided herein are methods of treating a patient having a cancer, the methods comprising (a) determining or having determined whether the patient's cancer has an NRG1 fusion; (b) selecting or having selected the patient for treatment with a quinazoline-based TKI and an anti-HER2/HER3 antibody when the patient's cancer has an NRG1 fusion; and (c) administering or having administered to the selected patient a combined therapeutically effective amount of a quinazoline-based TKI and an anti-HER2/HER3 antibody. In some aspects, step (a) comprises (i) obtaining or having obtained a biological sample from the patient; and (ii) performing or having performed an assay on the biological sample to determine the patient's cancer has an NRG1 fusion.

In one embodiment, provided herein are methods of treating a patient having a cancer, the methods comprising administering to the patient a combined therapeutically effective amount of a quinazoline-based TKI and an anti-HER2/HER3 antibody, wherein the cancer has an NRG1 fusion. In one embodiment, provided herein are compositions comprising a therapeutically effective amount of a quinazoline-based TKI and an anti-HER2/IER3 antibody, for use in the treatment of cancer in a patient, wherein the patient's cancer has an NRG1 fusion.

In one embodiment, provided herein are methods of selecting a patient having a cancer for treatment with a quinazoline-based TKI and an anti-HER2/IER3 antibody, the methods comprising (a) determining or having determined whether the patient's cancer has an NRG1 fusion; (b) selecting or having selected the patient for treatment with a quinazoline-based TKI and an anti-HER2/HER3 antibody when the patient's cancer has an NRG1 fusion. In some aspects, step (a) comprises (i) obtaining or having obtained a biological sample from the patient; and (ii) performing or having performed an assay on the biological sample to determine the patient's cancer has an NRG1 fusion. In some aspects, the methods further comprise (c) administering or having administered to the selected patient a combined therapeutically effective amount of a quinazoline-based TKI and an anti-HER2/IER3 antibody.

In some aspects of the embodiments, the NRG1 fusion is an NRG1-DOC4 fusion, an NRG1-VAMP2 fusion, an NRG1-CLU fusion, an NRG1-SLC3A2 fusion, an NRG1-CD74 fusion, an NRG1-ATP1B1 fusion, or an NRG1-SDC4 fusion.

In some aspects of the embodiments, the quinazoline-based TKI is IACS-015285, IACS-015296, IACS-070979, IACS-015293, IACS-070982, IACS-070863, IACS-070864, IACS-070871, IACS-070980, IACS-070968, IACS-070709, IACS-070989, or IACS-052336.

In some aspects of the embodiments, the methods further comprise administering to the patient an anti-HER2/IER3 antibody. In some aspects, the anti-HER2/IER3 antibody comprises trastuzumab, pertuzumab, or T-DM1.

In some aspects of the embodiments, the methods further comprise administering a further anti-cancer therapy to the patient. In some aspects, the further anti-cancer therapy is a surgical therapy, a chemotherapy, a radiation therapy, a cryotherapy, a hormonal therapy, a toxin therapy, an immunotherapy, or a cytokine therapy.

In some aspects of the embodiments, the cancer is a breast cancer, a lung cancer, a colorectal cancer, a neuroblastoma, a pancreatic cancer, a brain cancer, a stomach cancer, a skin cancer, a testicular cancer, a prostate cancer, an ovarian cancer, a liver cancer, an esophageal cancer, a cervical cancer, a head and neck cancer, a melanoma, or a glioblastoma. In some aspects, the cancer is a breast cancer or a lung cancer.

In some aspects of the embodiments, the patient has previously undergone at least one round of anti-cancer therapy. In some aspects of the embodiments, the methods further comprise reporting the presence of an NRG1 fusion in the patient's cancer. In some aspects, reporting comprises preparing a written or electronic report. In some aspects, the methods further comprise providing the report to the subject, a doctor, a hospital, or an insurance company.

As used herein, “essentially free,” in terms of a specified component, is used herein to mean that none of the specified component has been purposefully formulated into a composition and/or is present only as a contaminant or in trace amounts. The total amount of the specified component resulting from any unintended contamination of a composition is therefore well below 0.05%, preferably below 0.01%. Most preferred is a composition in which no amount of the specified component can be detected with standard analytical methods.

As used herein the specification, “a” or “an” may mean one or more. As used herein in the claim(s), when used in conjunction with the word “comprising,” the words “a” or “an” may mean one or more than one.

The use of the term “or” in the claims is used to mean “and/or” unless explicitly indicated to refer to alternatives only or the alternatives are mutually exclusive, although the disclosure supports a definition that refers to only alternatives and “and/or.” As used herein “another” may mean at least a second or more.

Throughout this application, the term “about” is used to indicate that a value includes the inherent variation of error for the device, the method being employed to determine the value, the variation that exists among the study subjects, or a value that is within 10% of a stated value.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1A. Representative dose response curves of MDA175-VII (NRG1-DOC4 fusion) treated with the indicated IACS novel quinazoline-based TKIs for 72 hours. Cell viability was determined by Cell Titer Glo Assay.

FIG. 1B. Bar graph of average ±SEM IC₅₀ values for MDA17-VII cell line treated with the indicated inhibitors for 72 hours.

FIG. 2. Bar graph of the mutant/WT EGFR ratio of MDA175-VII cells and Ba/F3 cells expressing WT EGFR.

FIG. 3A. Representative dose response curves of MDA175-VII (NRG1-DOC4 fusion) treated with the indicated anti-HER2 therapy with or without low dose treatment of IACS inhibitors for 72 hours. Cell viability was determined by Cell Titer Glo Assay.

FIG. 3B. Bar graph of average ±SEM IC₅₀ values for MDA17-VII cell line treated with the indicated inhibitors for 72 hours. Combinations with anti-HER2 antibody trastuzumab were not included in the bar graph because IC₅₀ values could not be calculated.

### DETAILED DESCRIPTION

Provided herein are methods for treating cancer patients with NRG1 fusions. In particular, the present methods comprise the administration of a quinazoline-based TKI, or a combination of a quinazoline-based TKI and an anti-HER2/IER3 antibody, to cancer patients identified as having an NRG1 fusion. In addition, the present methods comprise the identification and selection of cancer patients likely to benefit from the administration of a quinazoline-based TKI, or a combination of a quinazoline-based TKI and an anti-HER2/IER3 antibody, by determining whether the patient's cancer has an NRG1 fusion.

### I. NRG1 Fusions

An NRG1 fusion gene comprises at least a portion of the NRG1-gene fused to a sequence from a different chromosomal location. “At least a portion” indicates that the entire NRG1 gene may be present in a fusion or a portion thereof. The fusion may have at least the coding sequence of exons 6, 7, and 8 of NRG1. Another way to define the NRG1 portion in the NRG1-fusion gene is that it comprises the EGF-like domain of NRG1. The EGF-like domain is encoded by the 3′ end of the gene and is necessary for binding to ErbB-3. The NRG1-fusions retain an in-frame coding region for the EGF-like domain. The portion of the NRG1 gene may be fused to a sequence from a different chromosomal location such that the said sequence is located 5′ or 3′ to the portion of the NRG1 gene.

Preferably, the 3′ end of the NRG1-gene may be fused to a sequence from a different chromosomal location. In particular, the NRG1 fusion gene may be a fusion of the 3′ end of the NRG1-gene with the 5′ sequence of one of the genes selected from the group consisting of DOC4 (also known as Teneurin Transmembrane Protein 4 (TENM4); Protein Odd Oz/Ten-M Homolog 4; Tenascin-M4; Ten-M4; Ten-4; ODZ4; TNM4; Odz, Odd Oz/Ten-M Homolog 4 (Drosophila); Odz, Odd Oz\hat{ }en-M Homolog 4; Teneurin-4; KIAA1302; Doc4; ETM5; HGNC: 29945; Entrez Gene: 26011; Ensembl: ENSG00000149256; OMIM: 610084; and UniProtKB: Q6N022); CD74 (also known as CD74 Molecule; CD74 Antigen (Invariant Polypeptide Of Major Histocompatibility Complex, Class II Antigen-Associated); CD74 Molecule, Major Histocompatibility Complex, Class II Invariant Chain; HLA-DR Antigens-Associated Invariant Chain; Gamma Chain Of Class II Antigens; 1a-Associated Invariant Chain; MHC HLA-DR Gamma Chain; HLA-DR-Gamma; DHLAG; P33; HLA Class II Histocompatibility Antigen Gamma Chain; 1a Antigen-Associated Invariant Chain; 1a-GAMMA; HLADG; HGNC: 1697; Entrez Gene: 972; Ensembl: ENSG00000019582; OMIM: 142790, and UniProtKB: P04233); TNFRSF10B (also known as TNF Receptor Superfamily Member 10b; Tumor Necrosis Factor Receptor Superfamily, Member 10b; TNF-Related Apoptosis-Inducing Ligand Receptor 2; Death Receptor 5; TRAIL-R2; TRAILR2; KILLER; TRICK2; ZTNFR9; DR5; P53-Regulated DNA Damage inducible Cell Death Receptor (Killer); Tumor Necrosis Factor Receptor Superfamily Member 10B; Tumor Necrosis Factor Receptor-Like Protein ZTNFR9; Death Domain Containing Receptor For TRAIL/Apo-2L; Apoptosis Inducing Protein TRICK2A/2B; Apoptosis Inducing Receptor TRAIL-R2; Cytotoxic TRAIL Receptor-2; Fas-Like Protein; TRAIL Receptor 2; CD262 Antigen; KILLER/DR5; TRICK2A; TRICK2B; TRICKB; CD262; HGNC: 11905; Entrez Gene: 8795; Ensembl: ENSG00000120889; OMIM: 603612; and UniProtKB: 014763); CLU (also known as Clusterin; Testosterone-Repressed Prostate Message 2; Apolipoprotein J; Complement-Associated Protein SP-40,40; Complement Cytolysis Inhibitor; Complement Lysis Inhibitor; Sulfated Glycoprotein 2; Ku70-Binding Protein 1; NA1/NA2; TRPM-2; APO-J; APOJ; KUB1; CLI; Clusterin (Complement Lysis Inhibitor, SP-40,40, Sulfated Glycoprotein 2, Testosterone-Repressed Prostate Message 2, Apolipoprotein J); Aging-Associated Gene 4 Protein; Aging-Associated Protein 4; SGP-2; SP-40; TRPM2; AAG4; CLU1; CLU2; SGP2; HGNC: 2095; Entrez Gene: 1191; Ensembl: ENSG00000120885; OMIM: 185430; and UniProtKB: P10909); VAMP2 (also known as Vesicle Associated Membrane Protein 2; synaptobrevin 2; SYB2; Vesicle-Associated Membrane Protein 2; Synaptobrevin-2; HGNC: 12643; Entrez Gene: 6844; Ensembl: ENSG00000220205; OMIM: 185881; and UniProtKB: P63027); SLC3A2 (also known as Solute Carrier Family 3 Member 2; Lymphocyte Activation Antigen 4F2 Large Subunit; Solute Carrier Family 3 (Activators Of Dibasic And Neutral Amino Acid Transport), Member 2; Antigen Identified By Monoclonal Antibodies 4F2, TRA1.10, TROP4, And T43; Solute Carrier Family 3 (Amino Acid Transporter Heavy Chain), Member 2; 4F2 Cell-Surface Antigen Heavy Chain; CD98 Heavy Chain; 4F2HC; MDU1; Antigen Defined By Monoclonal Antibody 4F2, Heavy Chain; Antigen Defined By Monoclonal Antibody 4F2; 4F2 Heavy Cham Antigen; 4F2 Heavy Chain; CD98 Antigen; CD98HC; 4T2HC; NACAE; CD98; 4F2; HGNC: 11026; Entrez Gene: 6520; Ensembl: ENSG00000168003; OMIM: 158070; and UniProtKB: P08195); RBPMS (also known as RNA Binding Protein With Multiple Splicing; Heart And RRM Expressed Sequence; HERMES; RNA-Binding Protein With Multiple Splicing; RBP-MS; HGNC: 19097; Entrez Gene: 11030; Ensembl: ENSG00000157110; OMIM: 601558; and UniProtKB: Q93062); WRN (also known as Werner Syndrome RecQ Like Helicase; DNA Helicase, RecQ-Like Type 3; RecQ Protein-Like 2; Exonuclease WRN; RECQL2; RECQ3; Werner Syndrome ATP-Dependent Helicase; Werner Syndrome, RecQ Helicase-Like; Werner Syndrome; EC 3.6.4.12; EC 3.1.-.-; EC 3.6.1; RECQL3; HGNC: 12791; Entrez Gene: 7486; Ensembl: ENSG00000165392; OMIM: 604611 and UniProtKB: Q14191); SDC4 (also known as Syndecan 4 (Amphiglycan, Ryudocan); Syndecan Proteoglycan 4; Ryudocan Core Protein; Amphiglycan; SYND4; Ryudocan Amphiglycan; Syndecan-4; HGNC: 10661; Entrez Gene: 6385; Ensembl: ENSG00000124145; OMIM: 600017; and UniProtKB: P31431); KIF13B; SLECA2; PDE7A; ATP1B1; CDK1; BMPRIB; MCPH1; and RAB2IL1.

Certain embodiments of the present disclosure concern determining if a subject has an NRG1 fusion. Detection methods are known the art including PCR analyses, nucleic acid sequencing, fluorescence in situ hybridization (FISH), chromogenic in situ hybridization (CISH), and comparative genomic hybridization (CGH).

Samples that are suitable for use in the methods described herein contain genetic material, e.g., genomic DNA (gDNA). Genomic DNA is typically extracted from biological samples such as blood or mucosal scrapings of the lining of the mouth, but can be extracted from other biological samples including urine, tumor, or expectorant. The sample itself will typically include nucleated cells (e.g., blood or buccal cells) or tissue removed from the subject, including tumor tissue. Methods and reagents are known in the art for obtaining, processing, and analyzing samples. In some embodiments, the sample is obtained with the assistance of a health care provider, e.g., to draw blood or take a tumor biopsy. In some embodiments, the sample is obtained without the assistance of a health care provider, e.g., where the sample is obtained non-invasively, such as a sample comprising buccal cells that is obtained using a buccal swab or brush, or a mouthwash sample.

In particular, the patient sample can be any bodily tissue or fluid that includes nucleic acids from the cancer in the subject. In certain embodiments, the sample will be a blood sample comprising circulating tumor cells or cell-free DNA. In other embodiments, the sample can be a tissue, such as a tumor tissue. The tumor tissue may be fresh frozen or formalin-fixed, paraffin-embedded (FFPE).

In some cases, a biological sample may be processed for DNA isolation. For example, DNA in a cell or tissue sample can be separated from other components of the sample. Cells can be harvested from a biological sample using standard techniques known in the art. For example, cells can be harvested by centrifuging a cell sample and resuspending the pelleted cells. The cells can be resuspended in a buffered solution such as phosphate-buffered saline (PBS). After centrifuging the cell suspension to obtain a cell pellet, the cells can be lysed to extract DNA, e.g., gDNA. The sample can be concentrated and/or purified to isolate DNA. All samples obtained from a subject, including those subjected to any sort of further processing, are considered to be obtained from the subject. Routine methods can be used to extract genomic DNA from a biological sample, including, for example, phenol extraction. Alternatively, genomic DNA can be extracted with kits such as the QIAamp® Tissue Kit (Qiagen, Chatsworth, Calif.) or the Wizard® Genomic DNA purification kit (Promega).

Amplification of nucleic acids, where desirable, can be accomplished using methods known in the art, e.g., PCR. In one example, a sample (e.g., a sample comprising genomic DNA), is obtained from a subject. The DNA in the sample is then examined to determine the identity of an NRG1 fusion as described herein. An NRG1 fusion can be detected by any method described herein, e.g., by sequencing or by hybridization of the gene in the genomic DNA, RNA, or cDNA to a nucleic acid probe, e.g., a DNA probe (which includes cDNA and oligonucleotide probes) or an RNA probe. The nucleic acid probe can be designed to specifically or preferentially hybridize with a particular NRG1 fusion.

A set of probes typically refers to a set of primers, usually primer pairs, and/or detectably labeled probes that are used to detect the target genetic variations (e.g., NRG1 fusions) used in the actionable treatment recommendations of the present disclosure. The primer pairs are used in an amplification reaction to define an amplicon that corresponds to an NRG1 fusion. The set of amplicons are detected by a set of matched probes. In an exemplary embodiment, the present methods may use TaqMan™ (Roche Molecular Systems, Pleasanton, Calif.) assays that are used to detect a set of target genetic variations, such as NRG1 fusions. In one embodiment, the set of probes are a set of primers used to generate amplicons that are detected by a nucleic acid sequencing reaction, such as a next generation sequencing reaction. In these embodiments, for example, AmpliSEQ™ (Life Technologies/Ion Torrent, Carlsbad, Calif.) or TruSEQ™ (Illumina, San Diego, Calif.) technology can be employed.

Analysis of nucleic acid markers can be performed using techniques known in the art including, without limitation, sequence analysis, and electrophoretic analysis. Non-limiting examples of sequence analysis include Maxam-Gilbert sequencing, Sanger sequencing, capillary array DNA sequencing, thermal cycle sequencing, solid-phase sequencing, sequencing with mass spectrometry such as matrix-assisted laser desorption/ionization time-of-flight mass spectrometry (MALDI-TOF/MS), and sequencing by hybridization. Non-limiting examples of electrophoretic analysis include slab gel electrophoresis such as agarose or polyacrylamide gel electrophoresis, capillary electrophoresis, and denaturing gradient gel electrophoresis. Additionally, next generation sequencing methods can be performed using commercially available kits and instruments from companies such as the Life Technologies/Ion Torrent PGM or Proton, the Illumina HiSEQ or MiSEQ, and the Roche/454 next generation sequencing system.

Other methods of nucleic acid analysis can include direct manual sequencing (U.S. Pat. No. 5,288,644); automated fluorescent sequencing; single-stranded conformation polymorphism assays (SSCP); clamped denaturing gel electrophoresis (CDGE); two-dimensional gel electrophoresis (2DGE or TDGE); conformational sensitive gel electrophoresis (CSGE); denaturing gradient gel electrophoresis (DGGE); denaturing high performance liquid chromatography (DHPLC); infrared matrix-assisted laser desorption/ionization (IR-MALDI) mass spectrometry; mobility shift analysis; restriction enzyme analysis; quantitative real-time PCR; heteroduplex analysis; chemical mismatch cleavage (CMC); RNase protection assays; use of polypeptides that recognize nucleotide mismatches, e.g., E. coli mutS protein; allele-specific PCR, and combinations of such methods. See, e.g., U.S. Patent Publication No. 2004/0014095, which is incorporated herein by reference in its entirety.

In one example, a method of identifying an NRG1 fusion in a sample comprises contacting a nucleic acid from said sample with a nucleic acid probe that is capable of specifically hybridizing to a nucleic acid encoding an NRG1 fusion and detecting said hybridization. In a particular embodiment, said probe is detectably labeled such as with a radioisotope (³H, ³²P, or ³³P), a fluorescent agent (rhodamine, or fluorescein) or a chromogenic agent. In a particular embodiment, the probe is an antisense oligomer, for example PNA, morpholino-phosphoramidates, LNA or 2′-alkoxyalkoxy. The probe may be from about 8 nucleotides to about 100 nucleotides, or about 10 to about 75, or about 15 to about 50, or about 20 to about 30. In another aspect, said probes of the present disclosure are provided in a kit for identifying NRG1 fusions in a sample, said kit comprising oligonucleotides that specifically hybridize to specific NRG1 fusions. The kit may further comprise instructions for treating patients having tumors that contain NRG1 fusion with a quinazoline-based TKI, or a combination of a quinazoline-based TKI and an anti-HER2/IER3 antibody, based on the result of a hybridization test using the kit.

### I. Quinazoline-Based Tyrosine Kinase Inhibitors

Previous reports have also disclosed the design of novel quinazoline-based TKIs for inhibition of ErbB family members; however, these inhibitors have not been explored for use in inhibiting NRG-fusion cell lines. Exemplary quinazoline-based TKIs can be found in, for example, U.S. Ser. No. 62/838,702 and U.S. Ser. No. 62/838,696, each of which is incorporated herein by reference in its entirety.

The quinazoline-based tyrosine kinase inhibitor may be a compound having structural Formula (I):

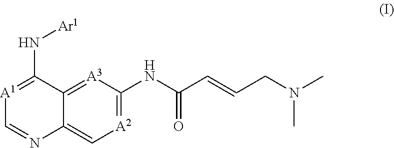

or a salt thereof, wherein:
A¹ is chosen from C(R¹) and N;
A² is chosen from C(R²) and N;
A³ is chosen from C(R³) and N;
Ar¹ is chosen from aryl and heteroaryl, either of which is optionally substituted with one or two R⁴ groups, and either of which is optionally substituted with one, two, or three R⁵ groups;
R¹ is chosen from halo, —CN, —OR⁶, —NR^{7a}R^{7b}, —COOR⁸, and —CONR^{9a}R^{9b};
R² and R³ are independently chosen from H, alkyl, and alkoxy;
each R⁴ is independently chosen from alkyl, haloalkyl, cycloalkyl, heterocycloalkyl, aryl, and heteroaryl, any of which is optionally substituted with one or two R¹⁰ groups;
each R⁵ is independently chosen from halo, —CN, —OR¹¹, —NR^{12a}R^{12b}, —COOR¹³, and —CONR^{14a}R^{14b};
each R⁶, R^{7a}, and R^{7b} is independently chosen from H, alkyl, haloalkyl, and C(═O)alkyl;
each R¹, R^{9a}, and R^{9b} is independently chosen from H and alkyl;
each R¹⁰ is independently chosen from halo, hydroxy, and alkoxy;
each R¹¹, R^{12a}, and R^{12b} is independently chosen from H, C₁₋₆alkyl, C₁₋₆halolkyl, and C(═O)C₁₋₆alkyl; and
each R¹³, R^{14a}, and R^{14b} is independently chosen from H and C₁₋₆alkyl.

In some cases, the compound has structural Formula (II):

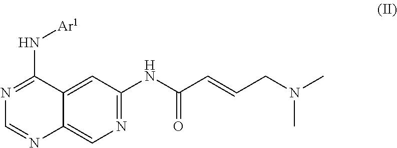

or a salt thereof, wherein:
Ar¹ is chosen from aryl and heteroaryl, either of which is optionally substituted with one or two R⁴ groups, and either of which is optionally substituted with one, two, or three R⁵ groups;
each R⁴ is independently chosen from alkyl, haloalkyl, cycloalkyl, heterocycloalkyl, aryl, and heteroaryl, any of which is optionally substituted with one or two R¹⁰ groups;
each R⁵ is independently chosen from halo, —CN, —OR¹¹, —NR^{12a}R^{12b}, —COOR¹³, and —CONR^{14a}R^{14b};
each R¹⁰ is independently chosen from halo, hydroxy, and alkoxy;
each R¹¹, R^{12a}, and R^{12b} is independently chosen from H, C₁₋₆alkyl, C₁₋₆halolkyl, and C(═O)C₁₋₆alkyl;
each R¹³, R^{14a}, and R^{14b} is independently chosen from H and C₁₋₆alkyl;
m and n are independently chosen from 1, 2, and 3; and
Y¹ is chosen from —NH— and —O—.

In some cases, Ar¹ is chosen from phenyl and monocyclic heteroaryl, either of which is optionally substituted with one or two R⁴ groups, and either of which is substituted with one, two, or three R⁵ groups. In some cases, Ar¹ is chosen from phenyl and monocyclic 6-membered heteroaryl, either of which is optionally substituted with one or two R⁴ groups, and either of which is substituted with one, two, or three R⁵ groups. In some cases, Ar¹ is chosen from phenyl, pyridyl, pyrimidyl, pyridazyl, and pyrazyl, any of which is optionally substituted with one or two R⁴ groups, and any of which is substituted with one, two, or three R⁵ groups. In some cases, Ar¹ is phenyl, and is optionally substituted with one or two R⁴ groups, and is substituted with one, two, or three R⁵ groups. In some cases, Ar¹ is chosen from pyridyl, pyrimidyl, pyridazyl, and pyrazyl, any of which is optionally substituted with one or two R⁴ groups, and any of which is substituted with one, two, or three R⁵ groups. In some cases, Ar¹ is pyridyl, and is optionally substituted with one or two R⁴ groups, and is substituted with one, two, or three R⁵ groups. In some cases, Ar¹ is chosen from pyrimidyl, pyridazyl, and pyrazyl, any of which is optionally substituted with one or two R⁴ groups, and any of which is substituted with one, two, or three R⁵ groups. In some cases, Ar¹ is chosen from naphthyl and bicyclic heteroaryl, either of which is optionally substituted with one or two R⁴ groups, and either of which is substituted with one, two, or three R⁵ groups. In some cases, Ar¹ is bicyclic heteroaryl, and is optionally substituted with one or two R⁴ groups, and is substituted with one, two, or three R⁵ groups. In some cases, Ar¹ is bicyclic 10-membered heteroaryl, and is optionally substituted with one or two R⁴ groups, and is substituted with one, two, or three R⁵ groups. In some cases, Ar¹ is chosen from quinolinyl and isoquinolinyl, either of which is optionally substituted with one or two R⁴ groups, and either of which is substituted with one, two, or three R⁵ groups. In some cases, Ar¹ is bicyclic 9-membered heteroaryl, and is optionally substituted with one or two R⁴ groups, and is substituted with one, two, or three R⁵ groups. In some cases, Ar¹ is chosen from indolyl, benzimidazolyl, benzopyrrolyl, benzoxazolyl, and benzisoxazolyl, any of which is optionally substituted with one or two R⁴ groups, and any of which is substituted with one, two, or three R⁵ groups. In some cases, Ar¹ is chosen from indolyl, benzimidazolyl, and benzopyrrolyl, any of which is optionally substituted with one or two R⁴ groups, and any of which is substituted with one, two, or three R⁵ groups.

In some cases, Ar¹ is optionally substituted with one R⁴ group. In some cases, Ar¹ is substituted with one or two R⁴ groups. In some cases, Ar¹ is substituted with one R⁴ group. In some cases, Ar¹ is substituted with two R⁴ groups. In some cases, each R⁴ is independently chosen from C₁₋₆alkyl, C₃₋₇cycloalkyl, 4- to 7-membered heterocycloalkyl, C₆₋₁₀aryl, and 6- to 10-membered heteroaryl, any of which is optionally substituted with one or two R¹⁰ groups. In some cases, each R⁴ is C₃₋₇cycloalkyl, and is optionally substituted with one or two R¹⁰ groups. In some cases, each R⁴ is C₁₋₆alkyl, and is optionally substituted with one or two R¹⁰ groups. In some cases, each R⁴ is C₁₋₆alkyl, and is optionally substituted with one or two R¹⁰ groups. In some cases, each R⁴ is independently chosen from C₃₋₇cycloalkyl and 4- to 7-membered heterocycloalkyl, either of which is optionally substituted with one or two R¹⁰ groups. In some cases, each R⁴ is independently chosen from C₆₋₁₀aryl and 6- to 10-membered heteroaryl, either of which is optionally substituted with one or two R¹⁰ groups. In some cases, each R⁴ is 6- to 10-membered heteroaryl and is optionally substituted with one or two R¹⁰ groups. In some cases, each R⁴ is monocyclic 5- to 7-membered heteroaryl and is optionally substituted with one or two R¹⁰ groups. In some cases, each R⁴ is chosen from pyrrolyl, pyrazolyl, imidazolyl, triazolyl, oxazolyl, and isoxazolyl, and is optionally substituted with one or two R¹⁰ groups. In some cases, each R⁴ is oxazolyl and is optionally substituted with one or two R¹⁰ groups. In some cases, each R⁴ is optionally substituted with one R¹⁰ group. In some cases, each R⁴ is substituted with one or two R¹⁰ groups. In some cases, each R⁴ is substituted with one R¹⁰ group. In some cases, R¹⁰ is halo. In some cases, R¹⁰ is hydroxy. In some cases, R¹⁰ is alkoxy. In some cases, R¹⁰ is C₁₋₆alkoxy. In some cases, each R⁴ is not substituted with an R¹⁰ group. In some cases, each R⁴ is cyclopropyl. In some cases, each R⁴ is cyclobutyl. In some cases, each R⁴ is C₁₋₆alkyl. In some cases, each R⁴ is methyl. In some cases, each R⁴ is hydroxyalkyl. In some cases, each R⁴ is hydroxymethyl. In some cases, Ar¹ is not substituted with an R⁴ group.

In some cases, Ar¹ is optionally substituted with one or two R⁵ groups. In some cases, Ar¹ is optionally substituted with one R⁵ group. In some cases, Ar¹ is substituted with one, two, or three R⁵ groups. In some cases, Ar¹ is substituted with one or two R⁵ groups. In some cases, Ar¹ is substituted with one R⁵ group. In some cases, each R⁵ is independently chosen from halo and cyano. In some cases, each R⁵ is independently chosen from —OR¹¹ and —NR^{12a}R^{12b}. In some cases, each R¹¹, R^{12a}, and R^{12b} is H. In some cases, each R⁵ is —OR¹¹. In some cases, each R¹¹ is alkyl. In some cases, each R¹¹ is C₁₋₆alkyl. In some cases, each R¹¹ is C₁₋₆haloalkyl. In some cases, each R¹¹ is halomethyl. In some cases, each R¹¹ is difluoromethyl. In some cases, each R¹¹ is trifluoromethyl. In some cases, each R¹¹, R^{12a}, and R^{12b} is C(═O)alkyl. In some cases, each R¹¹, R^{12a}, and R^{12b} is C(═O)C₁₋₆alkyl. In some cases, each R⁵ is independently chosen from —COOR¹³, and —CONR^{14a}R^{14b}. In some cases, each R¹³, R^{14a}, and R^{14b} is H. In some cases, each R¹³, R^{14a}, and R^{14b} is alkyl. In some cases, each R¹³, R^{14a}, and R^{14b} is C₁₋₆alkyl. In some cases, R⁵ is —COOR¹³. In some cases, R⁵ is —CONR^{14a}R^{14b}. In some cases, Ar¹ is not substituted with an R⁵ group. In some cases, Ar¹ is chosen from:

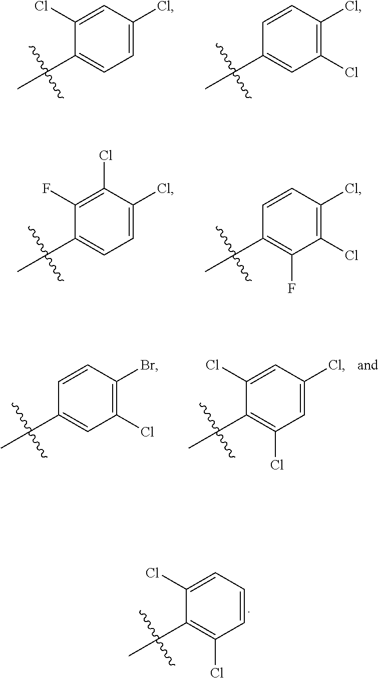

In some cases, Ar¹ is chosen from

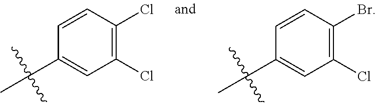 [SMILES: *c1ccc(Cl)c(Cl)c1 ; *c1ccc(Br)c(Cl)c1]

Also provided are embodiments wherein any embodiment above may be combined with any one or more of these embodiments, provided the combination is not mutually exclusive.

As used herein, two embodiments are “mutually exclusive” when one is defined to be something which is different than the other. For example, an embodiment wherein two groups combine to form a cycloalkyl is mutually exclusive with an embodiment in which one group is ethyl the other group is hydrogen. Similarly, an embodiment wherein one group is CH₂ is mutually exclusive with an embodiment wherein the same group is NH.

Also provided is a compound chosen from:

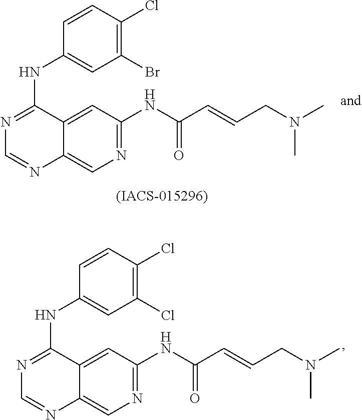 [SMILES: CN(C)C/C=C/C(=O)Nc1cc2c(Nc3ccc(Cl)c(Br)c3)ncnc2cn1 ; CN(C)C/C=C/C(=O)Nc1cc2c(Nc3ccc(Cl)c(Cl)c3)ncnc2cn1]

or a salt thereof.

The following schemes can be used to makes these compounds.

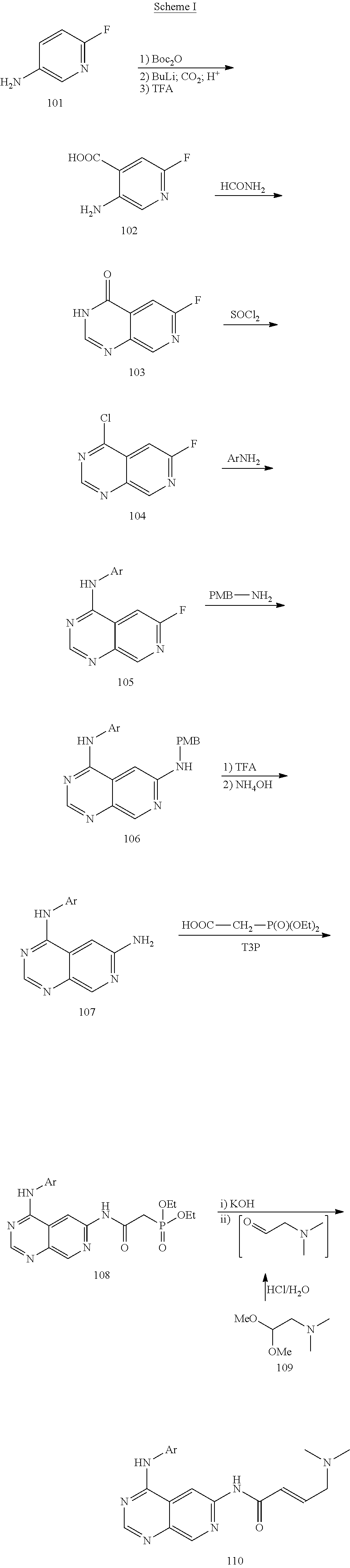

Pyridine derivative 101 is converted to isonicotinic acid derivative 102 via a three step protection/carboxylation/deprotection sequence. The bicyclic pyrido[3,4-d]-pyrimidine structure of 103 is then formed by condensation with formamide, which is then chlorinated to give dihalo compound 104. Sequential reaction of this intermediate with first ArNH₂, and then PMB-NH₂ (PMB=p-methoxybenzyl) gives disubstituted pyrido[3,4-d]-pyrimidine 106. The PMB group is removed under acidic conditions, and the free primary amine is then coupled with 2-(diethoxyphosphoryl)acetic acid to give amide 108. Reaction with 2-(dimethylamino)acetaldehyde (generated in situ from the acetal precursor 109) gives butenamide product 110.

For example, (E)-N-(4-((3-Bromo-4-chlorophenyl)amino)pyrido[3,4-d]pyrimidin-6-yl)-4-(dimethylamino)but-2-enamide can be made as follows.

### Step 1

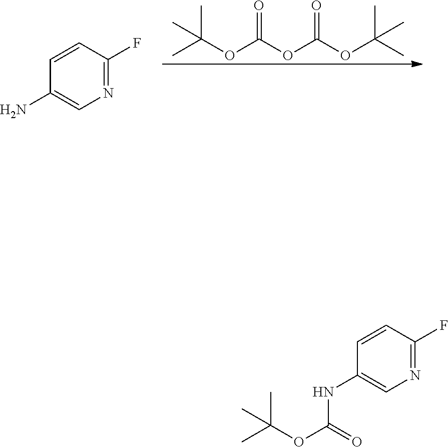

tert-Butyl (6-fluoropyridin-3-yl)carbamate To a solution of 6-fluoropyridin-3-amine (2.8 g, 25 mmol) in 6 mL of MTBE was added di-tert-butyl dicarbonate (21.8 g, 100 mmol) at room temperature. The mixture was stirred at 45° C. for 16 hrs. 1 gram of activated carbon was added and the mixture was stirred briefly and then filtered. The filtrate was purified by flash column chromatography eluting with PE/EA (2/1) to afford the title compound as a white solid (4.8 g, 90.6%). MS (ES+) C₁₀H₁₃FN₂O₂ requires: 212, found: 213 [M+H]⁺.

### Step 2

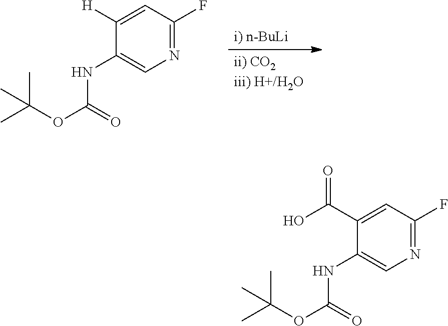 [SMILES: CC(C)(C)OC(=O)Nc1ccc(F)nc1 ; CC(C)(C)OC(=O)Nc1cnc(F)cc1C(=O)O]

5-((tert-Butoxycarbonyl)amino)-2-fluoroisonicotinic acid To a mixture of the product from the previous step (500 mg, 2.36 mmol), TMEDA (0.88 mL) and MTBE (7 mL) was added a solution of n-BuLi (2.5 M in hexane, 2.36 mL) at −70° C. After completion of the addition, the mixture was allowed to warm to −10° C. to −15° C. and held at this temperature for 3 h. Dry CO₂ gas was sparged at −70° C. for 2 h. The mixture was heated to 5° C. and then water (6 mL) was added. The aqueous phase was collected and the organic phase was extracted with 1 M NaOH. To the combined aqueous layers was added 6 M HCl slowly to adjust the pH to 2.5-3.0 The resulting mixture was extracted with EtOAc. The organic layer was dried and concentrated. The crude product was washed with a small amount of EtOAc to afford the title compound (340 mg, 56.2%) as a white solid. MS (ES+) C₁₁H₁₃FN₂O₄ requires: 256, found: 257 [M+H]⁺.

### Step 3

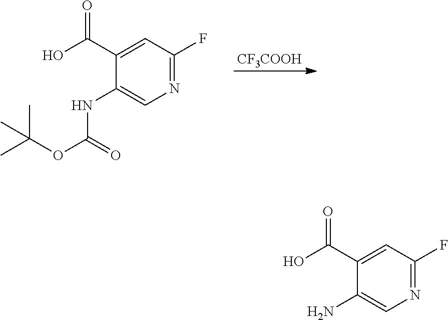 [SMILES: CC(C)(C)OC(=O)Nc1cnc(F)cc1C(=O)O ; "CF3COOH" ; Nc1cnc(F)cc1C(=O)O]

5-Amino-2-fluoroisonicotinic acid To a solution of the product from the previous step (1.9 g, 7.4 mmol) in DCM (8 mL) was added CF₃COOH (3.5 mL) at 0° C. The resulting solution was stirred at room temperature for 3 h. The mixture was concentrated under vacuum to afford the title compound as a yellow solid (900 mg, 77.8%). MS (ES+) C₆H₅FN₂O₂ requires: 156, found: 157 [M+H]⁺.

### Step 4

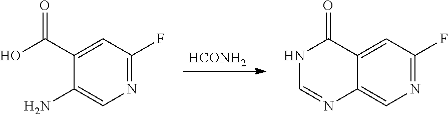

6-Fluoropyrido[3,4-d]pyrimidin-4(3H)-one A suspension of the product from the previous step (450 mg, 2.88 mmol) in formamide (5 mL) was heated at an internal temperature of 140° C. overnight with stirring. The mixture was cooled to room temperature, diluted with water (20 mL), and extracted with EtOAc. The organic layer was dried and concentrated. Water (5 mL) was added and the precipitate that formed was collected by filtration to afford the title compound (250 mg, 50.3%) as a yellow solid. MS (ES+) C₇H₄FN₃O, requires: 165, found: 166 [M+H]⁺.

### Step 5

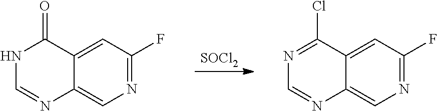

4-Chloro-6-fluoropyrido[3,4-d]pyrimidine A suspension of the product from the previous step (250 mg, 1.52 mmol) in SOCl₂ (5 mL) and DMF (1 drop) was refluxed for 2 h. The reaction mixture was evaporated to afford the title compound, which was used directly in the next step. MS (ES+) C₇H₃ClFN₃ requires: 183, found: 184 [M+H]⁺.

### Step 6

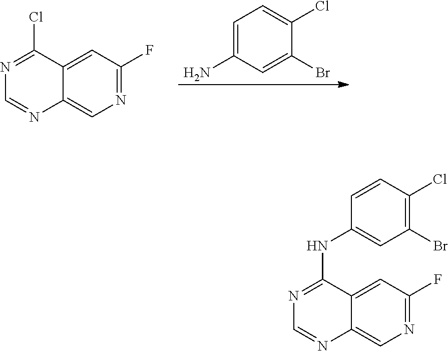 [SMILES: Fc1cc2c(Cl)ncnc2cn1 ; Nc1ccc(Cl)c(Br)c1 ; Fc1cc2c(Nc3ccc(Cl)c(Br)c3)ncnc2cn1]

N-(3-Bromo-4-chlorophenyl)-6-fluoropyrido[3,4-d]pyrimidin-4-amine A mixture of the product from the previous step (244 mg, 1.33 mmol) and 3-bromo-4-chloroaniline (301 mg, 1.46 mmol) in DMA (3 mL) was stirred at 30° C. for 16 hrs. The reaction was diluted with water and the pH was adjusted to ˜8 with sat. Na₂CO₃. PE was added and the mixture was stirred for 10 minutes. The solid was removed by filtration to afford the title compound as a brown solid (400 mg, 85.5%). MS (ES+) C₁₃H₇BrClFN₄ requires: 352, found: 353 [M+H]⁺.

### Step 7

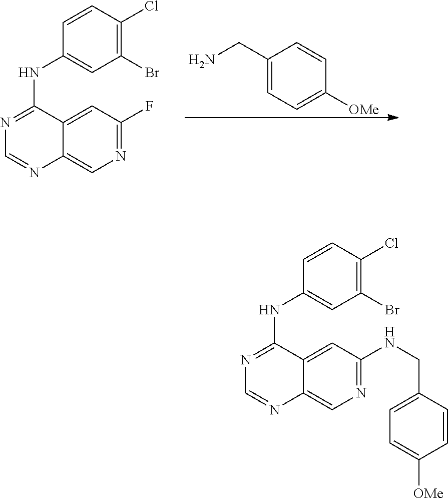 [SMILES: Fc1cc2c(Nc3ccc(Cl)c(Br)c3)ncnc2cn1 ; COc1ccc(CN)cc1 ; COc1ccc(CNc2cc3c(Nc4ccc(Cl)c(Br)c4)ncnc3cn2)cc1]

N⁴-(3-Bromo-4-chlorophenyl)-N⁶-(4-methoxybenzyl)pyrido[3,4-d]pyrimidine-4,6-diamine A mixture of the product from the previous step (365 mg, 1 mmol) and p-methoxybenzylamine (1.37 g, 10 mmol) in DMSO (5 mL) was stirred at 100° C. for 16 h. The reaction was then diluted with H₂O and extracted with EtOAc (20 mL×3). The combined organic layer was washed with brine, dried, and concentrated. The crude material was purified by flash column chromatography eluting with PE/EtOAc from 0% to 100% to afford the title compound as a yellow solid (280 mg, 52.5%). MS (ES+) C₂₁H₁₇BrClN₅O requires: 469, found: 470 [M+H]⁺.

### Step 8

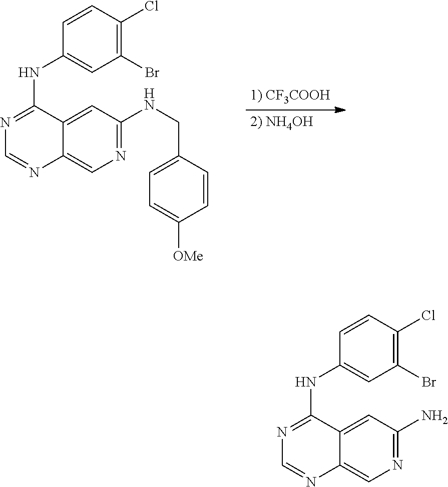 [SMILES: COc1ccc(CNc2cc3c(Nc4ccc(Cl)c(Br)c4)ncnc3cn2)cc1 ; Nc1cc2c(Nc3ccc(Cl)c(Br)c3)ncnc2cn1]

N⁴-(3-Bromo-4-chlorophenyl)pyrido[3,4-d]pyrimidine-4,6-diamine To a solution of the product from the previous step (280 mg, 0.6 mmol) in DCM (3 mL) was added CF₃COOH (1 mL). The resulting solution was stirred at room temperature for 16 h and then evaporated under vacuum to dryness. The residue was taken up in NH₄OH (2 mL) and stirred for 5 min. The solid was collected by filtration to afford the title compound as a yellow solid (160 mg, 76.9%). MS (ES+) C₁₃H₉BrClN₅ requires: 349, found: 350 [M+H]⁺.

### Step 9

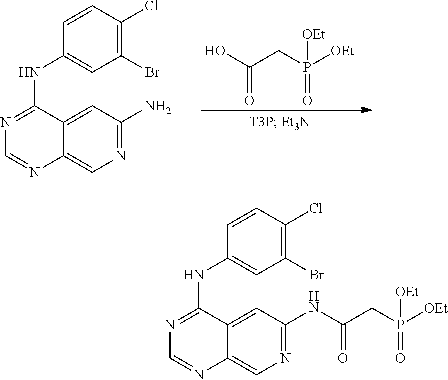 [SMILES: Nc1cc2c(Nc3ccc(Cl)c(Br)c3)ncnc2cn1 ; CCOP(=O)(CC(=O)O)OCC ; CCOP(=O)(CC(=O)Nc1cc2c(Nc3ccc(Cl)c(Br)c3)ncnc2cn1)OCC]

Diethyl (2-((4-((3-bromo-4-chlorophenyl)amino)pyrido[3,4-d]pyrimidin-6-yl)amino)-2-oxoethyl)phosphonate A mixture of the product from the previous step (150 mg, 0.43 mmol), 2-(diethoxyphosphoryl)acetic acid (126 mg, 0.64 mmol), T3P (409 mg, 0.64 mmol) and Et₃N (132 mg, 1.31 mmol) in EtOAc (3 mL) was stirred at 30° C. for 16 h. The reaction was diluted with H₂O. The solid that formed was removed by filtration and washed with EtOAc to afford the title compound as a beige solid (200 mg, 88.5%). MS (ES+) C₁₉H₂₀BrClN₅O₄P requires: 527, found: 528 [M+H]⁺.

### Step 10

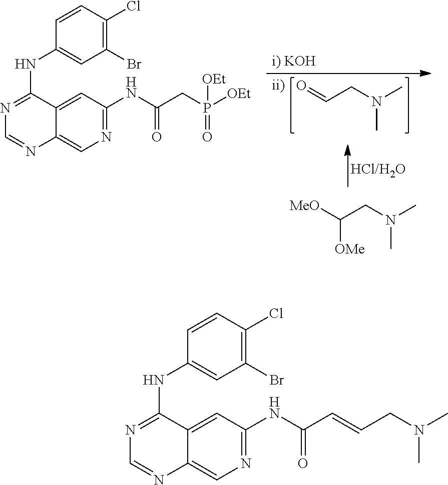

(E)-N-(4-((3-Bromo-4-chlorophenyl)amino)pyrido[3,4-d]pyrimidin-6-yl)-4-(dimethylamino)but-2-enamide To a solution of 2,2-dimethoxy-N,N-dimethylethan-1-amine (80 mg, 0.6 mmol) in 0.08 mL of H₂O was added 0.08 mL of 37% HCl. The solution was stirred at 40° C. for 20 hrs and then cooled to 0° C. This is called solution A. KOH (90 mg, 1.6 mmol) was dissolved in 0.4 mL of H₂O and cooled to 0° C. This is called solution B. To a solution of the product from the previous step (106 mg, 0.2 mmol) in 0.8 mL of THE and 0.4 mL of DMA was added LiCl (8 mg, 0.2 mmol) at 0° C. under Ar. The mixture was stirred at 0° C. for 15 min. Solution B was added and stirred at 0° C. for 2 min. Solution A was added and stirred for 2 hrs. H₂O (5 mL) and PE (5 mL) were added and the mixture was filtered to afford the title compound as a beige solid (70 mg, 60.9%).

MS (ES+) C₁₉H₁₈BrClN₆O requires: 460, found: 461 [M+H]⁺.

¹H NMR (500 MHz, DMSO) δ 10.98 (s, 1H), 10.39 (s, 1H), 9.03 (d, J=17.6 Hz, 2H), 8.68 (s, 1H), 8.28 (d, J=2.3 Hz, 1H), 7.92 (dd, J=8.8, 2.3 Hz, 1H), 7.67 (d, J=8.8 Hz, 1H), 6.88 (dt, J=15.4, 6.0 Hz, 1H), 6.53 (d, J=15.5 Hz, 1H), 3.11 (d, J=5.6 Hz, 2H), 2.20 (s, 6H).

In some embodiments, the quinazoline-based tyrosine kinase inhibitor may be a compound having structural Formula (I):

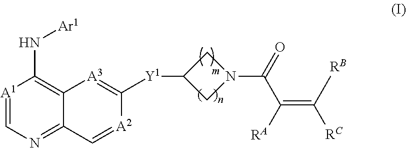

or a salt thereof, wherein:
A¹ is chosen from C(R¹) and N;
A² is chosen from C(R²) and N;
A³ is chosen from C(R³) and N;
Ar¹ is chosen from aryl and heteroaryl, either of which is optionally substituted with one or two R⁴ groups, and either of which is optionally substituted with one, two, or three R⁵ groups;
R^{A} and R^{B} are independently chosen from H and alkyl;
R^{C} is chosen from H, CH₃, and CH₂NR¹⁵R¹⁶;
R¹ is chosen from halo, —CN, —OR⁶, —NR^{7a}R^{7b}, —COOR⁸, and —CONR^{9a}R^{9b};
R² and R³ are independently chosen from H, alkyl, and alkoxy;
each R⁴ is independently chosen from alkyl, haloalkyl, cycloalkyl, heterocycloalkyl, aryl, and heteroaryl, any of which is optionally substituted with one or two R¹⁰ groups;
each R⁵ is independently chosen from halo, —CN, —OR¹¹, —NR^{12a}R^{12b}, —COOR¹³, and —CONR^{14a}R^{14b};
each R⁶, R^{7a}, and R^{7b} is independently chosen from H, alkyl, haloalkyl, and C(═O)alkyl;
each R¹, R^{9a}, and R^{9b} is independently chosen from H and alkyl;
each R¹⁰ is independently chosen from halo, hydroxy, and alkoxy;
each R¹¹, R^{12a}, and R^{12b} is independently chosen from H, C₁₋₆alkyl, C₁₋₆halolkyl, and C(═O)C₁₋₆alkyl;
each R¹³, R^{14a}, and R^{14b} is independently chosen from H and C₁₋₆alkyl;
R¹⁵ and R¹⁶ are independently chosen from H and C₁₋₆alkyl,
or R¹⁵ and R¹⁶, together with the nitrogen to which that they are both attached, combine to form a 5-7 membered heterocycloalkyl;
m and n are independently chosen from 1, 2, and 3; and
Y¹ is chosen from —NH— and —O—.

In some embodiments, the quinazoline-based tyrosine kinase inhibitor may be a compound having structural Formula (II):

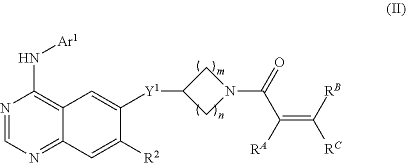

or a salt thereof, wherein:
Ar¹ is chosen from aryl and heteroaryl, either of which is optionally substituted with one or two R⁴ groups, and either of which is optionally substituted with one, two, or three R⁵ groups;
R^{A} and R^{B} are independently chosen from H and alkyl;
R^{C} is chosen from H, CH₃, and CH₂NR¹⁵R¹⁶;
R² is chosen from H, alkyl, and alkoxy;
each R⁴ is independently chosen from alkyl, haloalkyl, cycloalkyl, heterocycloalkyl, aryl, and heteroaryl, any of which is optionally substituted with one or two R¹⁰ groups;
each R⁵ is independently chosen from halo, —CN, —OR¹¹, —NR^{12a}R^{12b}, —COOR¹³, and —CONR^{14a}R^{14b};
each R¹⁰ is independently chosen from halo, hydroxy, and alkoxy;
each R¹¹, R^{12a}, and R^{12b} is independently chosen from H, C₁₋₆alkyl, C₁₋₆halolkyl, and C(═O)C₁₋₆alkyl;
each R¹³, R^{14a}, and R^{14b} is independently chosen from H and C₁₋₆alkyl;
R¹⁵ and R¹⁶ are independently chosen from H and C₁₋₆alkyl,
or R¹⁵ and R¹⁶, together with the nitrogen to which that they are both attached, combine to form a 5-7 membered heterocycloalkyl;
m and n are independently chosen from 1, 2, and 3; and
Y¹ is chosen from —NH— and —O—.

In some embodiments, the quinazoline-based tyrosine kinase inhibitor may be a compound having structural Formula (III):

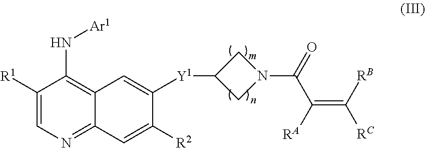

or a salt thereof, wherein:
Ar¹ is chosen from aryl and heteroaryl, either of which is optionally substituted with one or two R⁴ groups, and either of which is optionally substituted with one, two, or three R⁵ groups;
R^{A} and R^{B} are independently chosen from H and alkyl;
R^{C} is chosen from H, CH₃, and CH₂NR¹⁵R¹⁶;
R¹ is chosen from halo, —CN, —OR⁶, —NR^{7a}R^{7b}, —COOR⁸, and —CONR^{9a}R^{9b};
R² is chosen from H, alkyl, and alkoxy;
each R⁴ is independently chosen from alkyl, haloalkyl, cycloalkyl, heterocycloalkyl, aryl, and heteroaryl, any of which is optionally substituted with one or two R¹⁰ groups;
each R⁵ is independently chosen from halo, —CN, —OR¹¹, —NR^{12a}R^{12b}, —COOR¹³, and —CONR^{14a}R^{14b};
each R⁶, R^{7a}, and R^{7b} is independently chosen from H, alkyl, haloalkyl, and C(═O)alkyl;
each R¹, R^{9a}, and R^{9b} is independently chosen from H and alkyl;
each R¹⁰ is independently chosen from halo, hydroxy, and alkoxy;
each R¹¹, R^{12a}, and R^{12b} is independently chosen from H, C₁₋₆alkyl, C₁₋₆halolkyl, and C(═O)C₁₋₆alkyl;
each R¹³, R^{14a}, and R^{14b} is independently chosen from H and C₁₋₆alkyl;
R¹⁵ and R¹⁶ are independently chosen from H and C₁₋₆alkyl,
or R¹⁵ and R¹⁶, together with the nitrogen to which that they are both attached, combine to form a 5-7 membered heterocycloalkyl;
m and n are independently chosen from 1, 2, and 3; and
Y¹ is chosen from —NH— and —O—.

In some embodiments, the quinazoline-based tyrosine kinase inhibitor may be a compound having structural Formula (IV):

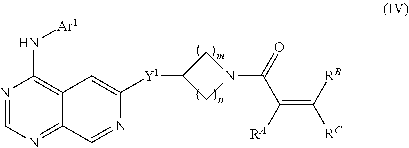

or a salt thereof, wherein:
Ar¹ is chosen from aryl and heteroaryl, either of which is optionally substituted with one or two R⁴ groups, and either of which is optionally substituted with one, two, or three R⁵ groups;
R^{A} and R^{B} are independently chosen from H and alkyl;
R^{C} is chosen from H, CH₃, and CH₂NR¹⁵R¹⁶;
each R⁴ is independently chosen from alkyl, haloalkyl, cycloalkyl, heterocycloalkyl, aryl, and heteroaryl, any of which is optionally substituted with one or two R¹⁰ groups;
each R⁵ is independently chosen from halo, —CN, —OR¹¹, —NR^{12a}R^{12b}, —COOR¹³, and —CONR^{14a}R^{14b};
each R¹⁰ is independently chosen from halo, hydroxy, and alkoxy;
each R¹¹, R^{12a}, and R^{12b} is independently chosen from H, C₁₋₆alkyl, C₁₋₆halolkyl, and C(═O)C₁₋₆alkyl;
each R¹³, R^{14a}, and R^{14b} is independently chosen from H and C₁₋₆alkyl;
R¹⁵ and R¹⁶ are independently chosen from H and C₁₋₆alkyl,
or R¹⁵ and R¹⁶, together with the nitrogen to which that they are both attached, combine to form a 5-7 membered heterocycloalkyl;
m and n are independently chosen from 1, 2, and 3; and
Y¹ is chosen from —NH— and —O—.

In some embodiments, the quinazoline-based tyrosine kinase inhibitor may be a compound having structural Formula (V):

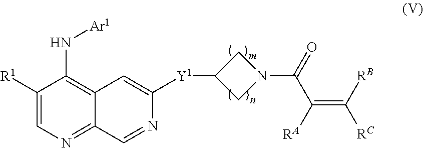

or a salt thereof, wherein:
Ar¹ is chosen from aryl and heteroaryl, either of which is optionally substituted with one or two R⁴ groups, and either of which is optionally substituted with one, two, or three R⁵ groups;
R^{A} and R^{B} are independently chosen from H and alkyl;
R^{C} is chosen from H, CH₃, and CH₂NR¹⁵R¹⁶;
R¹ is chosen from halo, —CN, —OR⁶, —NR^{7a}R^{7b}, —COOR⁸, and —CONR^{9a}R^{9b};
R² is chosen from H, alkyl, and alkoxy;
each R⁴ is independently chosen from alkyl, haloalkyl, cycloalkyl, heterocycloalkyl, aryl, and heteroaryl, any of which is optionally substituted with one or two R¹⁰ groups;
each R⁵ is independently chosen from halo, —CN, —OR¹¹, —NR^{12a}R^{12b}, —COOR¹³, and —CONR^{14a}R^{14b};
each R⁶, R^{7a}, and R^{7b} is independently chosen from H, alkyl, haloalkyl, and C(═O)alkyl;
each R¹, R^{9a}, and R^{9b} is independently chosen from H and alkyl;
each R¹⁰ is independently chosen from halo, hydroxy, and alkoxy;
each R¹¹, R^{12a}, and R^{12b} is independently chosen from H, C₁₋₆alkyl, C₁₋₆halolkyl, and C(═O)C₁₋₆alkyl;
each R¹³, R^{14a}, and R^{14b} is independently chosen from H and C₁₋₆alkyl;
R¹⁵ and R¹⁶ are independently chosen from H and C₁₋₆alkyl,
or R¹ and R¹⁶, together with the nitrogen to which that they are both attached, combine to form a 5-7 membered heterocycloalkyl;
m and n are independently chosen from 1, 2, and 3; and
Y¹ is chosen from —NH— and —O—.

In some cases, Ar¹ is chosen from phenyl and monocyclic heteroaryl, either of which is optionally substituted with one or two R⁴ groups, and either of which is optionally substituted with one, two, or three R⁵ groups. In some cases, Ar¹ is chosen from phenyl and monocyclic 6-membered heteroaryl, either of which is optionally substituted with one or two R⁴ groups, and either of which is optionally substituted with one, two, or three R⁵ groups. In some cases, Ar¹ is chosen from phenyl, pyridyl, pyrimidyl, pyridazyl, and pyrazyl, any of which is optionally substituted with one or two R⁴ groups, and any of which is optionally substituted with one, two, or three R⁵ groups. In some cases, Ar¹ is phenyl, and is optionally substituted with one or two R⁴ groups, and is optionally substituted with one, two, or three R⁵ groups. In some cases, Ar¹ is chosen from pyridyl, pyrimidyl, pyridazyl, and pyrazyl, any of which is optionally substituted with one or two R⁴ groups, and any of which is optionally substituted with one, two, or three R⁵ groups. In some cases, Ar¹ is pyridyl, and is optionally substituted with one or two R⁴ groups, and is optionally substituted with one, two, or three R⁵ groups. In some cases, Ar¹ is chosen from pyrimidyl, pyridazyl, and pyrazyl, any of which is optionally substituted with one or two R⁴ groups, and any of which is optionally substituted with one, two, or three R⁵ groups. In some cases, Ar¹ is chosen from naphthyl and bicyclic heteroaryl, either of which is optionally substituted with one or two R⁴ groups, and either of which is optionally substituted with one, two, or three R⁵ groups. In some cases, Ar¹ is bicyclic heteroaryl, and is optionally substituted with one or two R⁴ groups, and is optionally substituted with one, two, or three R⁵ groups. In some cases, Ar¹ is bicyclic 10-membered heteroaryl, and is optionally substituted with one or two R⁴ groups, and is optionally substituted with one, two, or three R⁵ groups. In some cases, Ar¹ is chosen from quinolinyl and isoquinolinyl, either of which is optionally substituted with one or two R⁴ groups, and either of which is optionally substituted with one, two, or three R⁵ groups. In some cases, Ar¹ is bicyclic 9-membered heteroaryl, and is optionally substituted with one or two R⁴ groups, and is optionally substituted with one, two, or three R⁵ groups. In some cases, Ar¹ is chosen from indolyl, benzimidazolyl, benzopyrrolyl, benzoxazolyl, and benzisoxazolyl, any of which is optionally substituted with one or two R⁴ groups, and any of which is optionally substituted with one, two, or three R⁵ groups. In some cases, Ar¹ is chosen from indolyl, benzimidazolyl, and benzopyrrolyl, any of which is optionally substituted with one or two R⁴ groups, and any of which is optionally substituted with one, two, or three R⁵ groups. In some cases, Ar¹ is optionally substituted with one R⁴ group. In some cases, Ar¹ is substituted with one or two R⁴ groups. In some cases, Ar¹ is substituted with one R⁴ group. In some cases, Ar¹ is substituted with two R⁴ groups. In some cases, each R⁴ is independently chosen from C₁₋₆alkyl, C₃₋₇ cycloalkyl, 4- to 7-membered heterocycloalkyl, C₆₋₁₀aryl, and 6- to 10-membered heteroaryl, any of which is optionally substituted with one or two R¹⁰ groups. In some cases, each R⁴ is C₃₋₇cycloalkyl, and is optionally substituted with one or two R¹⁰ groups. In some cases, each R⁴ is C₁₋₆alkyl, and is optionally substituted with one or two R¹⁰ groups. In some cases, each R⁴ is C₁₋₆alkyl, and is optionally substituted with one or two R¹⁰ groups. In some cases, each R⁴ is independently chosen from C₃₋₇cycloalkyl and 4- to 7-membered heterocycloalkyl, either of which is optionally substituted with one or two R¹⁰ groups. In some cases, each R⁴ is independently chosen from C₆₋₁₀aryl and 6- to 10-membered heteroaryl, either of which is optionally substituted with one or two R¹⁰ groups. In some cases, each R⁴ is 6- to 10-membered heteroaryl and is optionally substituted with one or two R¹⁰ groups. In some cases, each R⁴ is monocyclic 5- to 7-membered heteroaryl and is optionally substituted with one or two R¹⁰ groups. In some cases, each R⁴ is chosen from pyrrolyl, pyrazolyl, imidazolyl, triazolyl, oxazolyl, and isoxazolyl, and is optionally substituted with one or two R¹⁰ groups. In some cases, each R⁴ is oxazolyl and is optionally substituted with one or two R¹⁰ groups. In some cases, each R⁴ is optionally substituted with one R¹⁰ group. In some cases, each R⁴ is substituted with one or two R¹⁰ groups. In some cases, each R⁴ is substituted with one R¹⁰ group. In some cases, R¹⁰ is halo. In some cases, R¹⁰ is hydroxy. In some cases, R¹⁰ is alkoxy. In some cases, R¹⁰ is C₁₋₆alkoxy. In some cases, each R⁴ is not substituted with an R¹⁰ group. In some cases, each R⁴ is cyclopropyl. In some cases, each R⁴ is cyclobutyl. In some cases, each R⁴ is C₁₋₆alkyl. In some cases, each R⁴ is methyl. In some cases, each R⁴ is hydroxyalkyl. In some cases, each R⁴ is hydroxymethyl. In some cases, Ar¹ is not substituted with an R⁴ group. In some cases, Ar¹ is optionally substituted with one or two R⁵ groups. In some cases, Ar¹ is optionally substituted with one R⁵ group. In some cases, Ar¹ is optionally substituted with one, two, or three R⁵ groups. In some cases, Ar¹ is substituted with one or two R⁵ groups. In some cases, Ar¹ is substituted with one R⁵ group. In some cases, each R⁵ is independently chosen from halo and cyano. In some cases, each R⁵ is independently chosen from —OR¹¹ and —NR^{12a}R^{12b}. In some cases, each R¹¹, R^{12a}, and R^{12b} is H. In some cases, each R⁵ is —OR¹¹. In some cases, each R¹¹ is alkyl. In some cases, each R¹¹ is C₁₋₆alkyl. In some cases, each R¹¹ is C₁₋₆haloalkyl. In some cases, each R¹¹ is halomethyl. In some cases, each R¹¹ is difluoromethyl. In some cases, each R¹¹ is trifluoromethyl. In some cases, each R¹¹, R^{12a}, and R^{12b} is C(═O)alkyl. In some cases, each R¹¹, R^{12a}, and R^{12b} is C(═O)C₁₋₆alkyl. In some cases, each R⁵ is independently chosen from —COOR¹³, and —CONR^{14a}R^{14b}. In some cases, each R¹³, R^{14a}, and R^{14b} is H. In some cases, each R¹³, R^{14a}, and R^{14b} is alkyl. In some cases, each R¹³, R^{14a}, and R^{14b} is C₁₋₆alkyl. In some cases, R⁵ is —COOR¹³. In some cases, R⁵ is —CONR^{14a}R^{14b}. In some cases, Ar¹ is not substituted with an R⁵ group. In some cases, Ar¹ is chosen from:

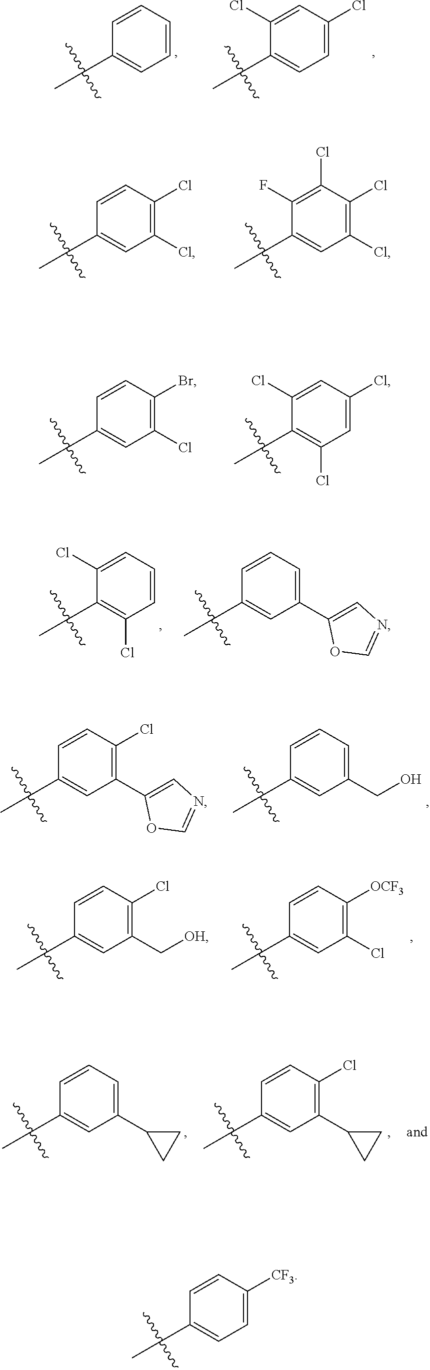

In some cases, Ar¹ is chosen from:

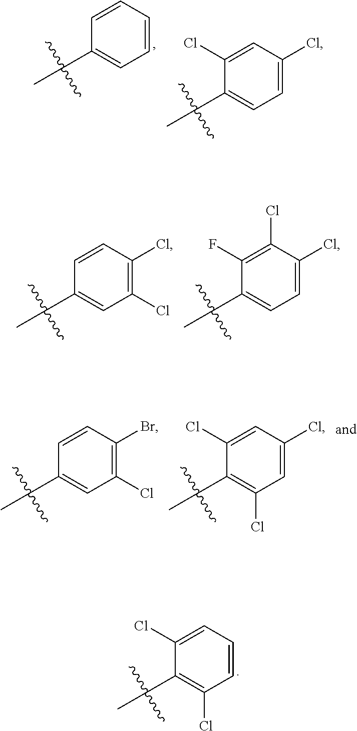

In some cases, Ar¹ is

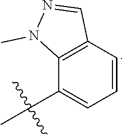 [SMILES: *c1cccc2cnn(C)c12]

In some cases, m is 1 and n is 1, m is 2 and n is 2, or m is 1 and n is 3. In some cases, m is 1 and n is 1, or m is 2 and n is 2. In some cases, m is 1. In some cases, m is 2. In some cases, m is 3. In some cases, n is 1. In some cases, n is 2. In some cases, n is 3.

In some cases, Y¹ is —NH—. In some cases, Y¹ is —O—. In some cases, R^{A} and R^{B} are independently chosen from H and C₁₋₆alkyl. In some cases, R^{A} is H. In some cases, R^{A} is C₁₋₆alkyl. In some cases, R^{B} is H. In some cases, R^{B} is C₁₋₆alkyl. In some cases, R^{C} is H. In some cases, R^{C} is CH₃. In some cases, R^{C} is CH₂NR¹⁵R¹⁶. In some cases, R¹⁵ and R¹⁶ are independently chosen from H and C₁₋₆alkyl. In some cases, R¹⁵ and R¹⁶ are independently chosen from H and methyl. In some cases, R¹⁵ and R¹⁶ are C₁₋₆alkyl. In some cases, R¹⁵ and R¹⁶ are methyl. In some cases, at least one of R¹⁵ and R¹⁶ is H. In some cases, R¹⁵ and R¹⁶ are H. In some cases, R¹⁵ and R¹⁶, together with the nitrogen to which that they are both attached, combine to form a 5-7 membered heterocycloalkyl. In some cases, R¹⁵ and R¹⁶, together with the nitrogen to which that they are both attached, combine to form a 5-7 membered heterocycloalkyl chosen from pyrrolidine, piperidine, piperazine, and morpholine.

In some embodiments, the quinazoline-based tyrosine kinase inhibitor may be a compound having structural Formula (VI):

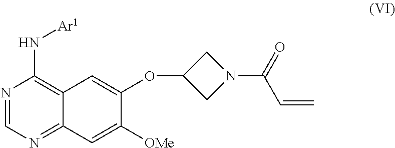

or a salt thereof, wherein:
Ar¹ is chosen from aryl and heteroaryl, either of which is optionally substituted with one, two, or three R⁵ groups;
each R⁵ is independently chosen from halo, —CN, —OR¹¹, —NR^{12a}R^{12b}, —COOR¹³, and —CONR^{14a}R^{14b};
each R¹¹, R^{12a}, and R^{12b} is independently chosen from H, C₁₋₆alkyl, C₁₋₆halolkyl, and C(═O)C₁₋₆alkyl; and
each R¹³, R^{14a}, and R^{14b} is independently chosen from H and C₁₋₆alkyl.

In some cases, Ar¹ is phenyl, and is substituted with one, two, or three R⁵ groups. In some cases, R⁵ is halo. In some cases, Ar¹ is chosen from

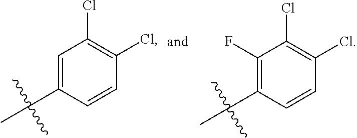 [SMILES: *c1ccc(Cl)c(Cl)c1 ; *c1ccc(Cl)c(Cl)c1F]

In some embodiments, the quinazoline-based tyrosine kinase inhibitor may be a compound having structural Formula (VII):

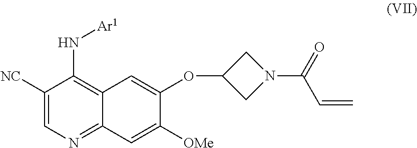

or a salt thereof, wherein:
Ar¹ is chosen from aryl and heteroaryl, either of which is optionally substituted with one, two, or three R⁵ groups;
each R⁵ is independently chosen from halo, —CN, —OR¹¹, —NR^{12a}R^{12b}, —COOR¹³, and —CONR^{14a}R^{14b};
each R¹¹, R^{12a}, and R^{12b} is independently chosen from H, C₁₋₆alkyl, C₁₋₆halolkyl, and C(═O)C₁₋₆alkyl; and
each R¹³, R^{14a}, and R^{14b} is independently chosen from H and C₁₋₆alkyl.

In some cases, Ar¹ is phenyl, and is substituted with one, two, or three R⁵ groups. In some cases, R⁵ is halo. In some cases, Ar¹ is chosen from

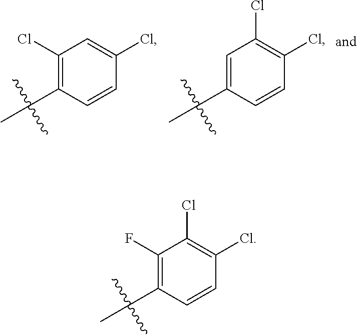 [SMILES: *c1ccc(Cl)cc1Cl ; *c1ccc(Cl)c(Cl)c1 ; *c1ccc(Cl)c(Cl)c1F]

In some embodiments, the quinazoline-based tyrosine kinase inhibitor may be a compound having structural Formula (VIII):

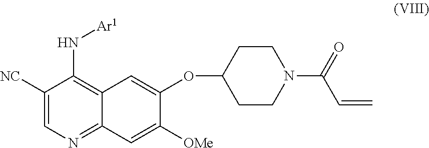

or a salt thereof, wherein:
Ar¹ is chosen from aryl and heteroaryl, either of which is optionally substituted with one, two, or three R⁵ groups;
each R⁵ is independently chosen from halo, —CN, —OR¹¹, —NR^{12a}R^{12b}, —COOR¹³, and —CONR^{14a}R^{14b};
each R¹¹, R^{12a}, and R^{12b} is independently chosen from H, C₁₋₆alkyl, C₁₋₆halolkyl, and C(═O)C₁₋₆alkyl; and
each R¹³, R^{14a}, and R^{14b} is independently chosen from H and C₁₋₆alkyl.

In some cases, Ar¹ is phenyl, and is substituted with one, two, or three R⁵ groups. In some cases, R⁵ is halo. In some cases, Ar¹ is

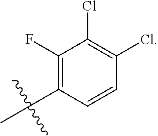 [SMILES: *c1ccc(Cl)c(Cl)c1F]

In some cases, each R⁵ is independently chosen from halo, —CN, and —OR¹¹. In some cases, each R⁵ is independently chosen from halo and —CN. In some cases, each R⁵ is halo. In some cases, Ar¹ is chosen from phenyl and monocyclic heteroaryl, either of which is optionally substituted with one, two, or three R⁵ groups. In some cases, Ar¹ is chosen from phenyl, pyridyl, pyrimidyl, pyridazyl, and pyrazyl, any of which is optionally substituted with one, two, or three R⁵ groups. In some cases, Ar¹ is phenyl, and is substituted with one, two, or three R⁵ groups. In some cases, Ar¹ is substituted with one or two R⁵ groups. In some cases, Ar¹ is substituted with one R⁵ group. In some cases, Ar¹ is chosen from

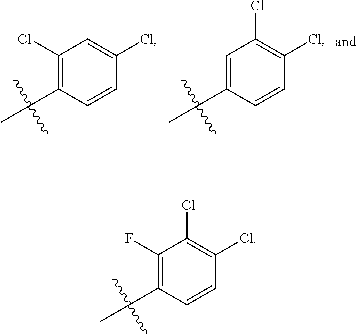 [SMILES: *c1ccc(Cl)cc1Cl ; *c1ccc(Cl)c(Cl)c1 ; *c1ccc(Cl)c(Cl)c1F]

In some embodiments, the quinazoline-based tyrosine kinase inhibitor may be a compound chosen from:

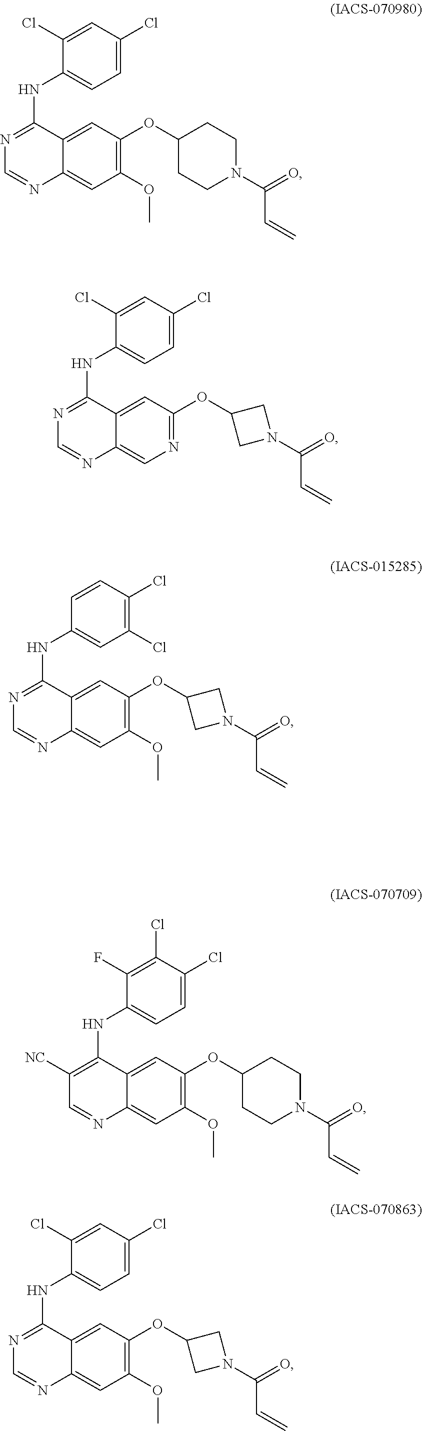 [SMILES: C=CC(=O)N1CCC(Oc2cc3c(Nc4ccc(Cl)cc4Cl)ncnc3cc2OC)CC1 ; C=CC(=O)N1CC(Oc2cc3c(Nc4ccc(Cl)cc4Cl)ncnc3cn2)C1 ; C=CC(=O)N1CC(Oc2cc3c(Nc4ccc(Cl)c(Cl)c4)ncnc3cc2OC)C1 ; C=CC(=O)N1CCC(Oc2cc3c(Nc4ccc(Cl)c(Cl)c4F)c(C#N)cnc3cc2OC)CC1 ; C=CC(=O)N1CC(Oc2cc3c(Nc4ccc(Cl)cc4Cl)ncnc3cc2OC)C1]

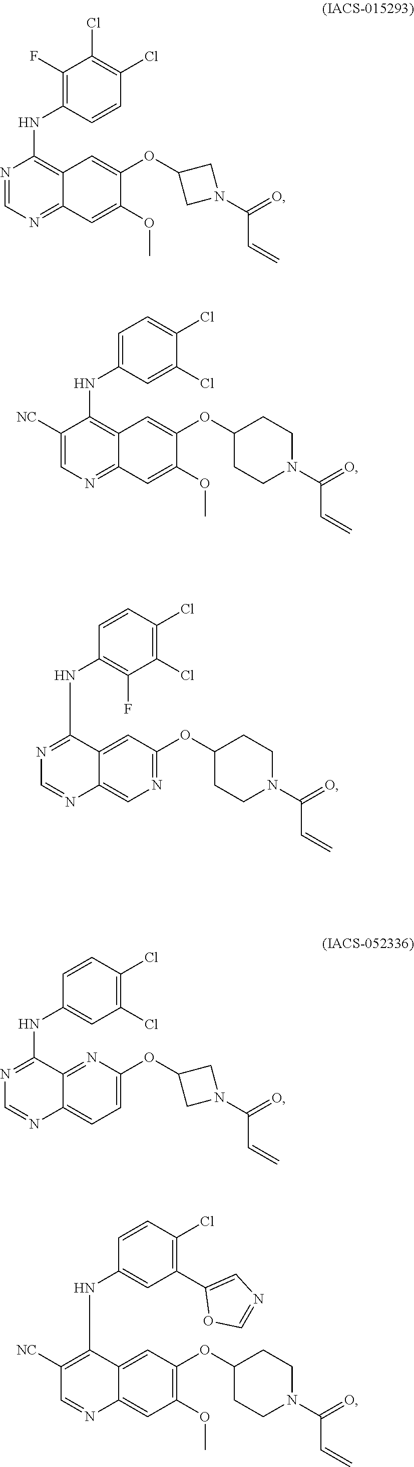 [SMILES: C=CC(=O)N1CC(Oc2cc3c(Nc4ccc(Cl)c(Cl)c4F)ncnc3cc2OC)C1 ; C=CC(=O)N1CCC(Oc2cc3c(Nc4ccc(Cl)c(Cl)c4)c(C#N)cnc3cc2OC)CC1 ; C=CC(=O)N1CCC(Oc2cc3c(Nc4ccc(Cl)c(Cl)c4F)ncnc3cn2)CC1 ; C=CC(=O)N1CC(Oc2ccc3ncnc(Nc4ccc(Cl)c(Cl)c4)c3n2)C1 ; C=CC(=O)N1CCC(Oc2cc3c(Nc4ccc(Cl)c(-c5cnco5)c4)c(C#N)cnc3cc2OC)CC1]

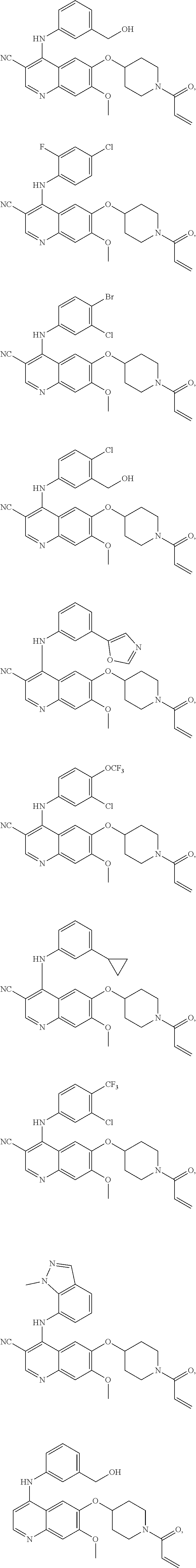

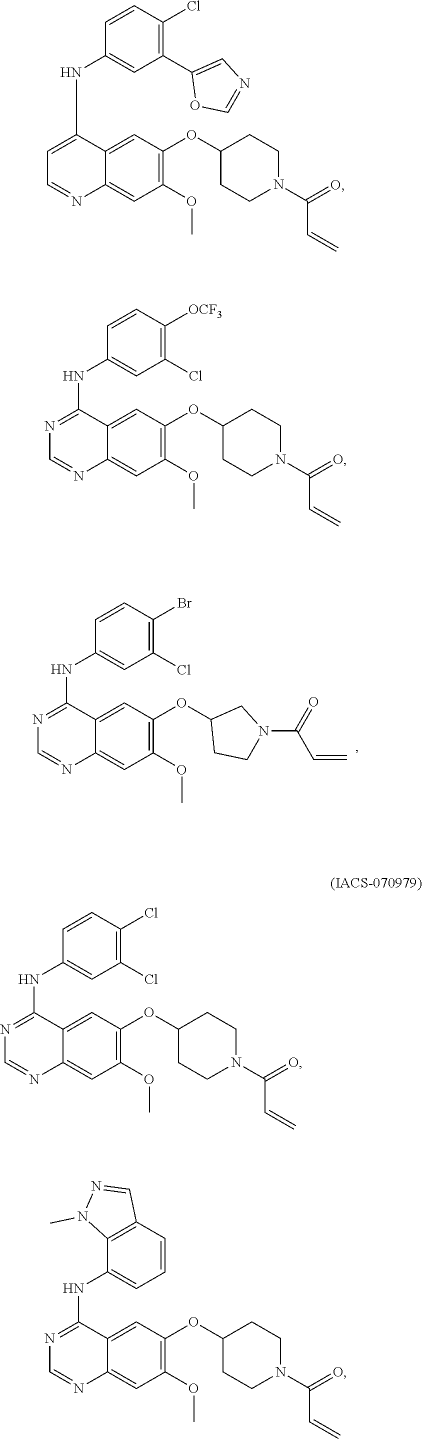 [SMILES: C=CC(=O)N1CCC(Oc2cc3c(Nc4ccc(Cl)c(-c5cnco5)c4)ccnc3cc2OC)CC1 ; C=CC(=O)N1CCC(Oc2cc3c(Nc4ccc(OC(F)(F)F)c(Cl)c4)ncnc3cc2OC)CC1 ; C=CC(=O)N1CCC(Oc2cc3c(Nc4ccc(Br)c(Cl)c4)ncnc3cc2OC)C1 ; C=CC(=O)N1CCC(Oc2cc3c(Nc4ccc(Cl)c(Cl)c4)ncnc3cc2OC)CC1 ; C=CC(=O)N1CCC(Oc2cc3c(Nc4cccc5cnn(C)c45)ncnc3cc2OC)CC1]

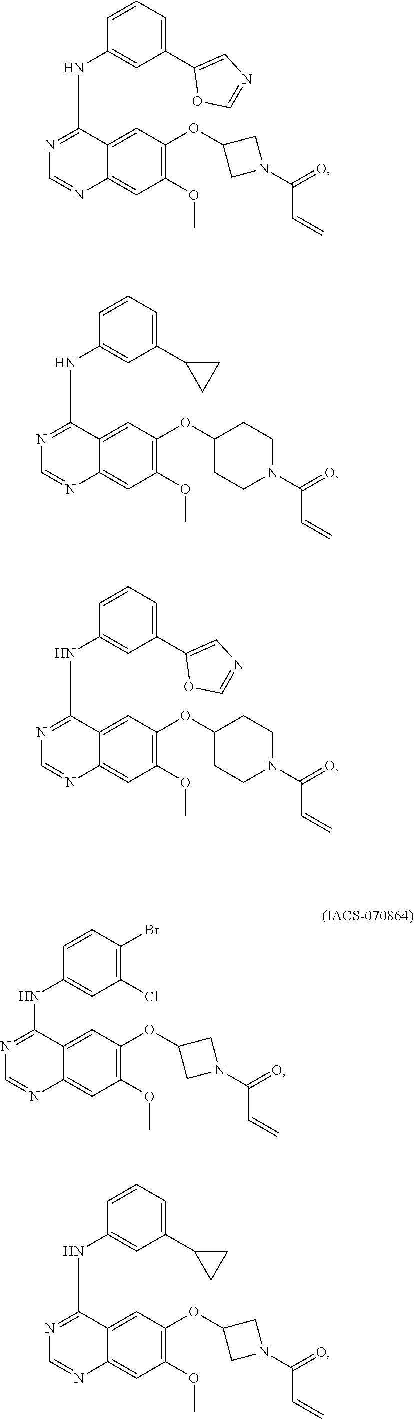 [SMILES: C=CC(=O)N1CC(Oc2cc3c(Nc4cccc(-c5cnco5)c4)ncnc3cc2OC)C1 ; C=CC(=O)N1CCC(Oc2cc3c(Nc4cccc(C5CC5)c4)ncnc3cc2OC)CC1 ; C=CC(=O)N1CCC(Oc2cc3c(Nc4cccc(-c5cnco5)c4)ncnc3cc2OC)CC1 ; C=CC(=O)N1CC(Oc2cc3c(Nc4ccc(Br)c(Cl)c4)ncnc3cc2OC)C1 ; C=CC(=O)N1CC(Oc2cc3c(Nc4cccc(C5CC5)c4)ncnc3cc2OC)C1]

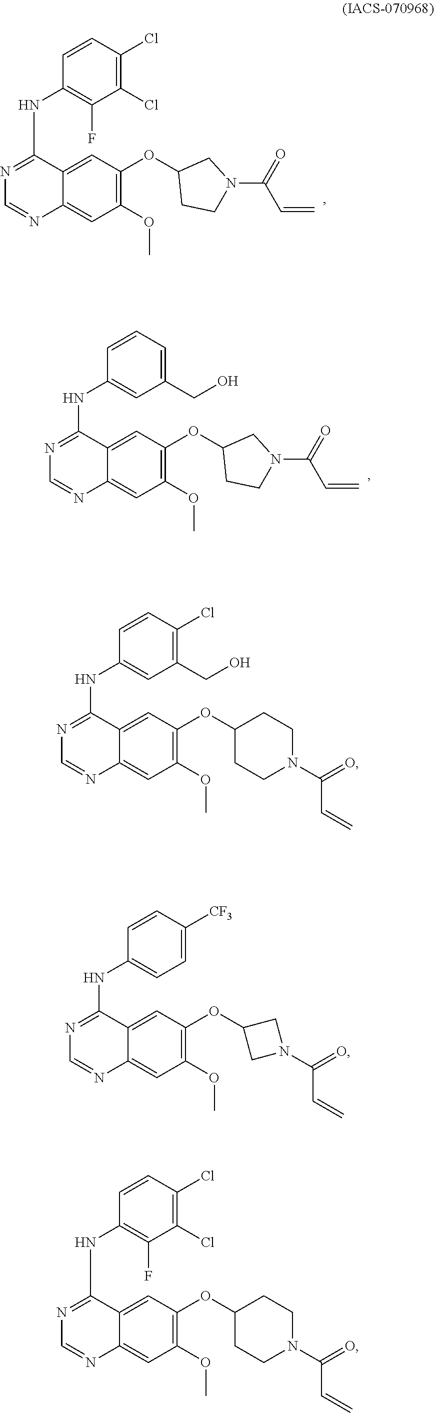 [SMILES: C=CC(=O)N1CCC(Oc2cc3c(Nc4ccc(Cl)c(Cl)c4F)ncnc3cc2OC)C1 ; C=CC(=O)N1CCC(Oc2cc3c(Nc4cccc(CO)c4)ncnc3cc2OC)C1 ; C=CC(=O)N1CCC(Oc2cc3c(Nc4ccc(Cl)c(CO)c4)ncnc3cc2OC)CC1 ; C=CC(=O)N1CC(Oc2cc3c(Nc4ccc(C(F)(F)F)cc4)ncnc3cc2OC)C1 ; C=CC(=O)N1CCC(Oc2cc3c(Nc4ccc(Cl)c(Cl)c4F)ncnc3cc2OC)CC1]

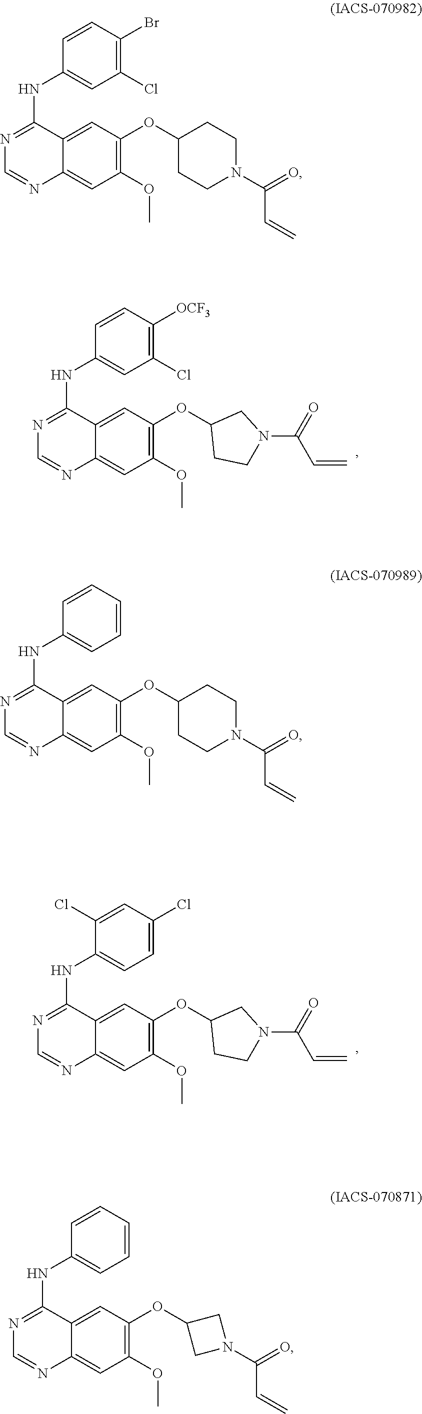 [SMILES: C=CC(=O)N1CCC(Oc2cc3c(Nc4ccc(Br)c(Cl)c4)ncnc3cc2OC)CC1 ; C=CC(=O)N1CCC(Oc2cc3c(Nc4ccc(OC(F)(F)F)c(Cl)c4)ncnc3cc2OC)C1 ; C=CC(=O)N1CCC(Oc2cc3c(Nc4ccccc4)ncnc3cc2OC)CC1 ; C=CC(=O)N1CCC(Oc2cc3c(Nc4ccc(Cl)cc4Cl)ncnc3cc2OC)C1 ; C=CC(=O)N1CC(Oc2cc3c(Nc4ccccc4)ncnc3cc2OC)C1]

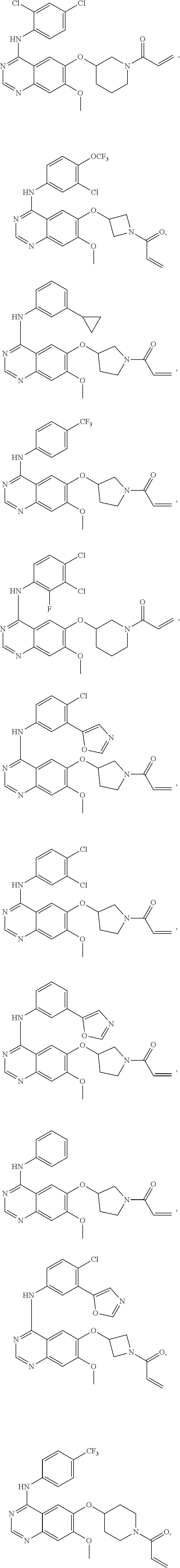

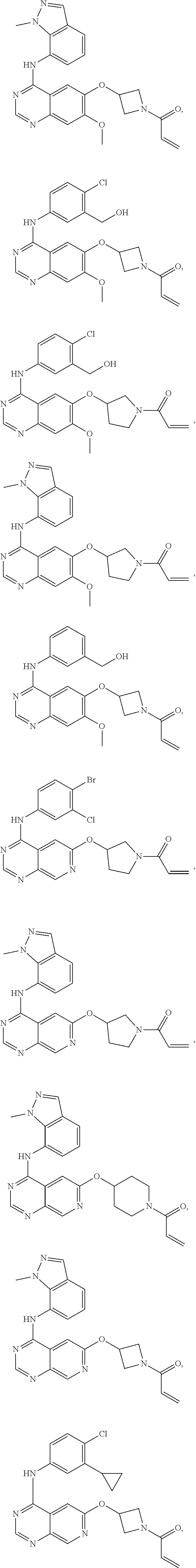

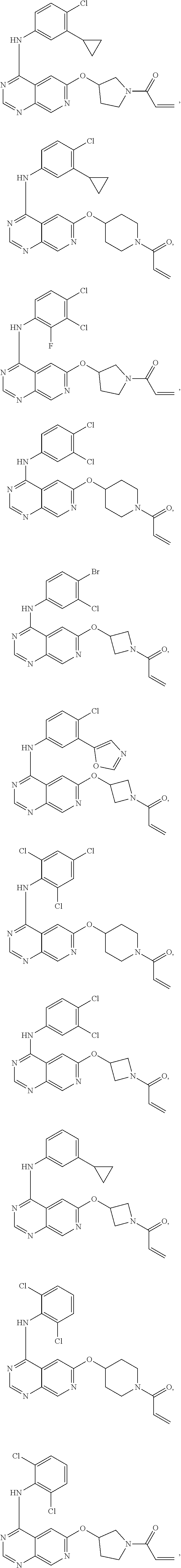

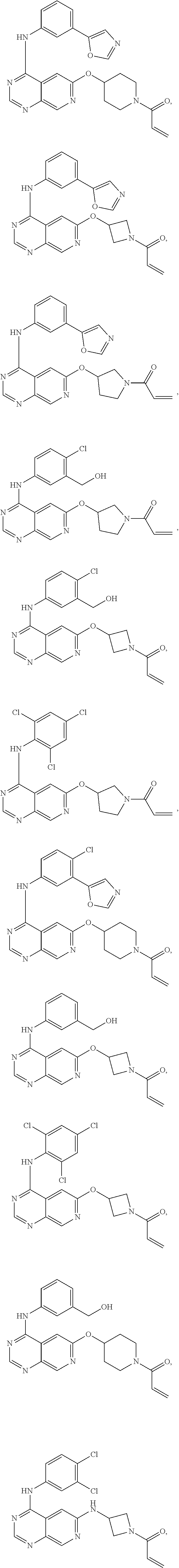

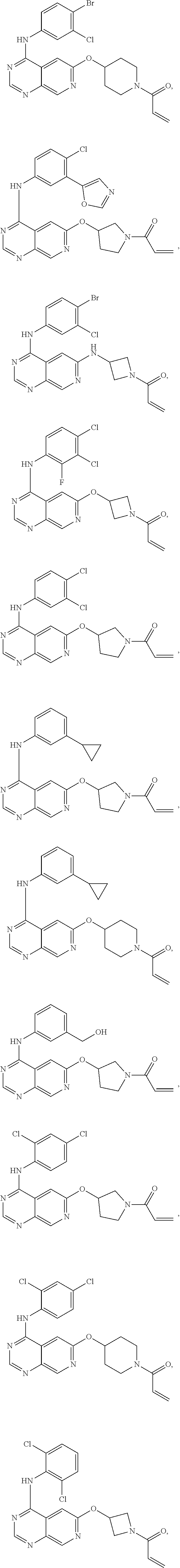

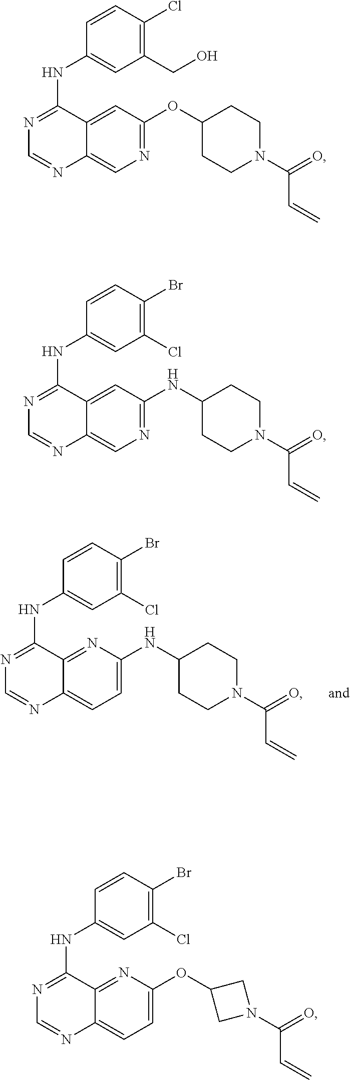 [SMILES: C=CC(=O)N1CCC(Oc2cc3c(Nc4ccc(Cl)c(CO)c4)ncnc3cn2)CC1 ; C=CC(=O)N1CCC(Nc2cc3c(Nc4ccc(Br)c(Cl)c4)ncnc3cn2)CC1 ; C=CC(=O)N1CCC(Nc2ccc3ncnc(Nc4ccc(Br)c(Cl)c4)c3n2)CC1 ; "and" ; C=CC(=O)N1CC(Oc2ccc3ncnc(Nc4ccc(Br)c(Cl)c4)c3n2)C1]

or a salt thereof.

In some embodiments, the quinazoline-based tyrosine kinase inhibitor may be a compound chosen from:

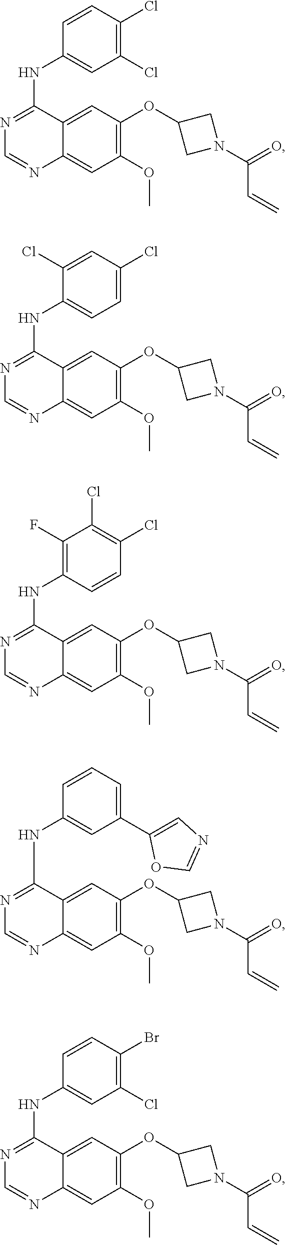

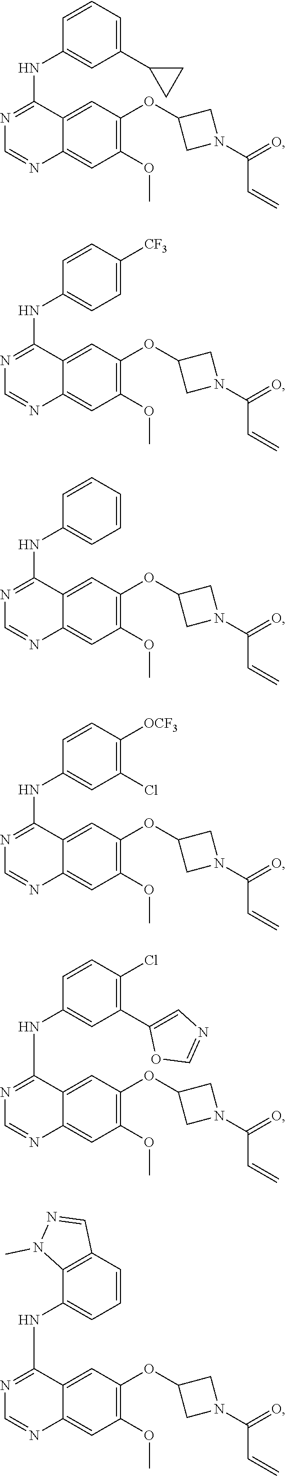

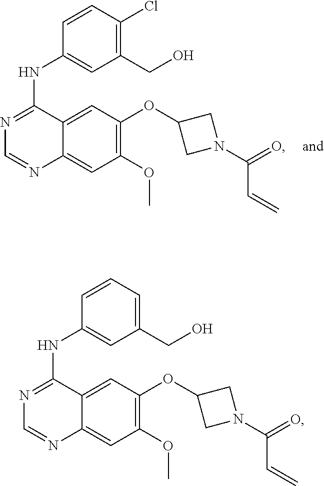 [SMILES: C=CC(=O)N1CC(Oc2cc3c(Nc4ccc(Cl)c(CO)c4)ncnc3cc2OC)C1 ; C=CC(=O)N1CC(Oc2cc3c(Nc4cccc(CO)c4)ncnc3cc2OC)C1]

or a salt thereof.

In some embodiments, the quinazoline-based tyrosine kinase inhibitor may be a compound chosen from:

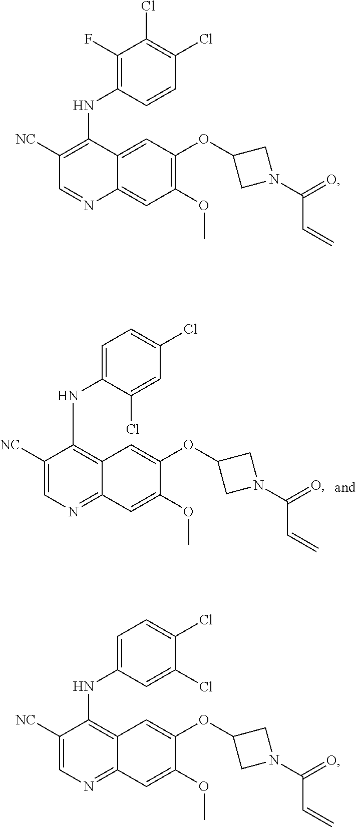 [SMILES: C=CC(=O)N1CC(Oc2cc3c(Nc4ccc(Cl)c(Cl)c4F)c(C#N)cnc3cc2OC)C1 ; C=CC(=O)N1CC(Oc2cc3c(Nc4ccc(Cl)cc4Cl)c(C#N)cnc3cc2OC)C1 ; C=CC(=O)N1CC(Oc2cc3c(Nc4ccc(Cl)c(Cl)c4)c(C#N)cnc3cc2OC)C1]

or a salt thereof.

In some embodiments, the quinazoline-based tyrosine kinase inhibitor may be a compound chosen from:

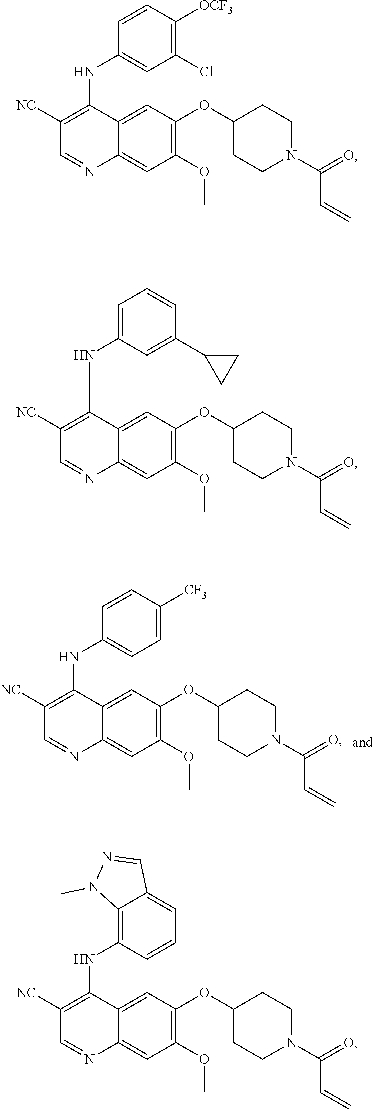 [SMILES: C=CC(=O)N1CCC(Oc2cc3c(Nc4ccc(OC(F)(F)F)c(Cl)c4)c(C#N)cnc3cc2OC)CC1 ; C=CC(=O)N1CCC(Oc2cc3c(Nc4cccc(C5CC5)c4)c(C#N)cnc3cc2OC)CC1 ; C=CC(=O)N1CCC(Oc2cc3c(Nc4ccc(C(F)(F)F)cc4)c(C#N)cnc3cc2OC)CC1 ; C=CC(=O)N1CCC(Oc2cc3c(Nc4cccc5cnn(C)c45)c(C#N)cnc3cc2OC)CC1]

or a salt thereof.

The following schemes can be used to practice the present disclosure.

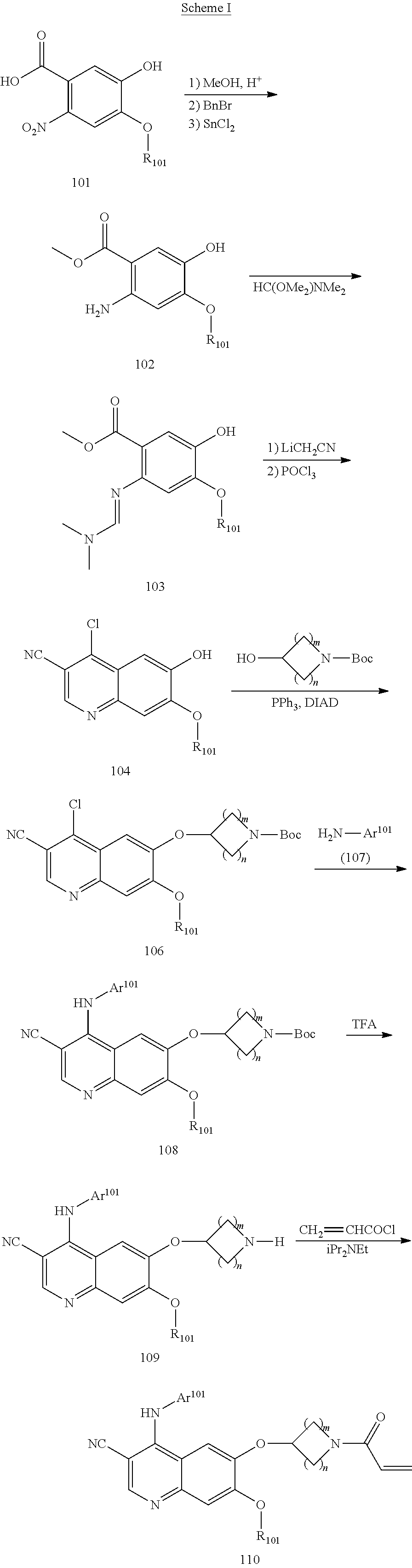

The functional groups of starting material 101 are manipulated via sequential Fisher esterification, Williamson ether formation, and nitro group reduction to give functionalized benzene 102. Condensation with DMF dimethyl acetal gives amidine 103, which is converted to chloro quinoline 104 via ring formation with acetonitrile anion, followed by chlorination of the intermediate quinolone compound (not shown). Mitsunobu-type coupling of secondary alcohol 105 with phenol 104 provides ether 106. S_{N}Ar reaction with arylamine 107 gives the substitution product 108. After removal of the Boc protecting group, secondary amine 109 is reacted with acryloyl chloride to give amide 107.

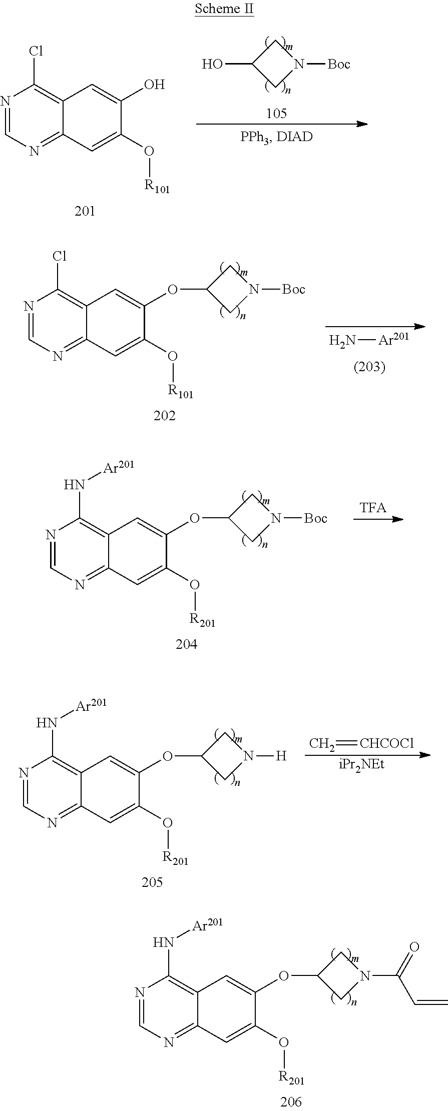

Synthesis proceeds as for Scheme I, with the difference being the choice of quinazoline starting material 201.

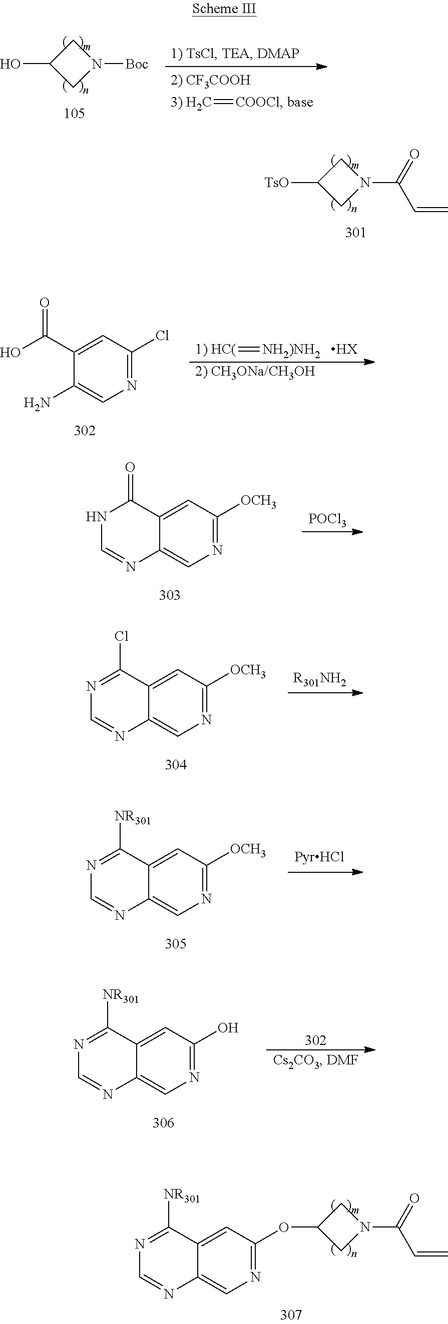

Heterocyclic tosylate 301 is prepared in three steps from Boc-protected hydroxy cycloamine 105 (Scheme I). Anthranilic acid analogue 302 is converted into a bicyclic arene with formamidine, followed by displacement of the choride to form phenolic ether 303. Reaction with phosphorus oxychloride converts the amide functionality to chloro compound 304. Reaction with R₃₀₁NH₂ gives aminoarene 305. The methoxy group is removed under acidic conditions to give phenol 306. Finally, reaction of the phenol with tosylate 301 under Williamson ether synthesis conditions gives 307.

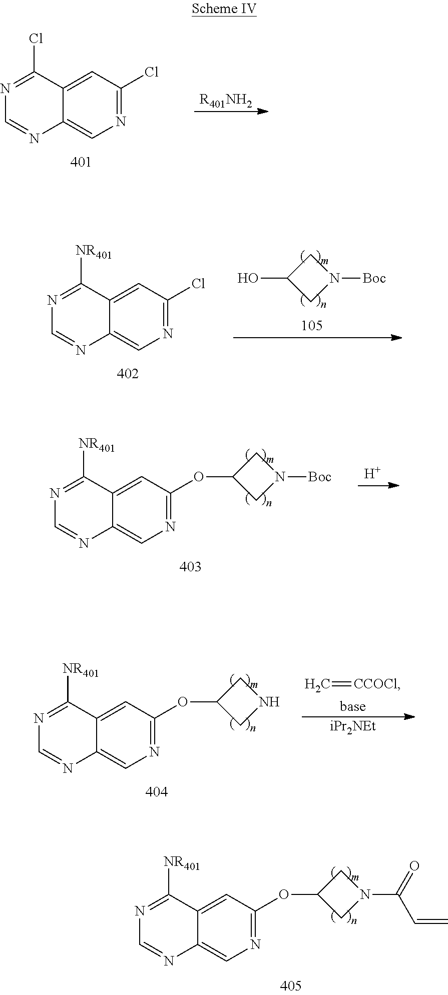

Pyrido[3,4-d]pyrimidine derivative 401 is reacted selectively at the 4-position to give aniline compound 402. Reaction with hydroxy cycloamine 105 (Scheme 1) gives ether 403. The Boc protecting group is removed under acidic conditions to give secondary amine 404, which is then reacted with acryloyl chloride to give amide 405.

When ranges of values are disclosed, and the notation “from n₁ . . . to n₂” or “between n₁ . . . and n₂” is used, where n₁ and n₂ are the numbers, then unless otherwise specified, this notation is intended to include the numbers themselves and the range between them. This range may be integral or continuous between and including the end values. By way of example, the range “from 2 to 6 carbons” is intended to include two, three, four, five, and six carbons, since carbons come in integer units. Compare, by way of example, the range “from 1 to 3 μM (micromolar),” which is intended to include 1 μM, 3 μM, and everything in between to any number of significant figures (e.g., 1.255 μM, 2.1 μM, 2.9999 μM, etc.).

The term “about,” as used herein, is intended to qualify the numerical values which it modifies, denoting such a value as variable within a margin of error. When no particular margin of error, such as a standard deviation to a mean value given in a chart or table of data, is recited, the term “about” should be understood to mean that range which would encompass the recited value and the range which would be included by rounding up or down to that figure as well, taking into account significant figures.

The term “acyl,” as used herein, alone or in combination, refers to a carbonyl attached to an alkenyl, alkyl, aryl, cycloalkyl, heteroaryl, heterocycle, or any other moiety were the atom attached to the carbonyl is carbon. An “acetyl” group refers to a —C(O)CH₃ group. An “alkylcarbonyl” or “alkanoyl” group refers to an alkyl group attached to the parent molecular moiety through a carbonyl group. Examples of such groups include methylcarbonyl and ethylcarbonyl. Examples of acyl groups include formyl, alkanoyl and aroyl.

The term “alkenyl,” as used herein, alone or in combination, refers to a straight-chain or branched-chain hydrocarbon radical having one or more double bonds and containing from 2 to 20 carbon atoms. In certain embodiments, said alkenyl will comprise from 2 to 6 carbon atoms. The term “alkenylene” refers to a carbon-carbon double bond system attached at two or more positions such as ethenylene [(—CH═CH—),(—C::C—)]. Examples of suitable alkenyl radicals include ethenyl, propenyl, 2-methylpropenyl, 1,4-butadienyl and the like. Unless otherwise specified, the term “alkenyl” may include “alkenylene” groups.

The term “alkoxy,” as used herein, alone or in combination, refers to an alkyl ether radical, wherein the term alkyl is as defined below. Examples of suitable alkyl ether radicals include methoxy, ethoxy, n-propoxy, isopropoxy, n-butoxy, iso-butoxy, sec-butoxy, tert-butoxy, and the like.

The term “alkyl,” as used herein, alone or in combination, refers to a straight-chain or branched-chain alkyl radical containing from 1 to 20 carbon atoms. In certain embodiments, said alkyl will comprise from 1 to 10 carbon atoms. In further embodiments, said alkyl will comprise from 1 to 8 carbon atoms. Alkyl groups may be optionally substituted as defined herein. Examples of alkyl radicals include methyl, ethyl, n-propyl, isopropyl, n-butyl, isobutyl, sec-butyl, tert-butyl, pentyl, iso-amyl, hexyl, octyl, noyl and the like. The term “alkylene,” as used herein, alone or in combination, refers to a saturated aliphatic group derived from a straight or branched chain saturated hydrocarbon attached at two or more positions, such as methylene (—CH₂—). Unless otherwise specified, the term “alkyl” may include “alkylene” groups.

The term “alkylamino,” as used herein, alone or in combination, refers to an alkyl group attached to the parent molecular moiety through an amino group. Suitable alkylamino groups may be mono- or dialkylated, forming groups such as, for example, N-methylamino, N-ethylamino, N,N-dimethylamino, N,N-ethylmethylamino and the like.

The term “alkylidene,” as used herein, alone or in combination, refers to an alkenyl group in which one carbon atom of the carbon-carbon double bond belongs to the moiety to which the alkenyl group is attached.

The term “alkylthio,” as used herein, alone or in combination, refers to an alkyl thioether (R—S—) radical wherein the term alkyl is as defined above and wherein the sulfur may be singly or doubly oxidized. Examples of suitable alkyl thioether radicals include methylthio, ethylthio, n-propylthio, isopropylthio, n-butylthio, iso-butylthio, sec-butylthio, tert-butylthio, methanesulfonyl, ethanesulfinyl, and the like.

The term “alkynyl,” as used herein, alone or in combination, refers to a straight-chain or branched chain hydrocarbon radical having one or more triple bonds and containing from 2 to 20 carbon atoms. In certain embodiments, said alkynyl comprises from 2 to 6 carbon atoms. In further embodiments, said alkynyl comprises from 2 to 4 carbon atoms. The term “alkynylene” refers to a carbon-carbon triple bond attached at two positions such as ethynylene (—C:::C—, —C≡C—). Examples of alkynyl radicals include ethynyl, propynyl, hydroxypropynyl, butyn-1-yl, butyn-2-yl, pentyn-1-yl, 3-methylbutyn-1-yl, hexyn-2-yl, and the like. Unless otherwise specified, the term “alkynyl” may include “alkynylene” groups.

The terms “amido” and “carbamoyl,” as used herein, alone or in combination, refer to an amino group as described below attached to the parent molecular moiety through a carbonyl group, or vice versa. The term “C-amido” as used herein, alone or in combination, refers to a —C(O)N(RR′) group with R and R′ as defined herein or as defined by the specifically enumerated “R” groups designated. The term “N-amido” as used herein, alone or in combination, refers to a RC(O)N(R′)— group, with R and R′ as defined herein or as defined by the specifically enumerated “R” groups designated. The term “acylamino” as used herein, alone or in combination, embraces an acyl group attached to the parent moiety through an amino group. An example of an “acylamino” group is acetylamino (CH₃C(O)NH—).

The term “amino,” as used herein, alone or in combination, refers to —NRR′, wherein R and R′ are independently chosen from hydrogen, alkyl, acyl, heteroalkyl, aryl, cycloalkyl, heteroaryl, and heterocycloalkyl, any of which may themselves be optionally substituted. Additionally, R and R′ may combine to form heterocycloalkyl, either of which may be optionally substituted.

The term “aryl,” as used herein, alone or in combination, means a carbocyclic aromatic system containing one, two or three rings wherein such polycyclic ring systems are fused together. The term “aryl” embraces aromatic groups such as phenyl, naphthyl, anthracenyl, and phenanthryl.

The term “arylalkenyl” or “aralkenyl,” as used herein, alone or in combination, refers to an aryl group attached to the parent molecular moiety through an alkenyl group.

The term “arylalkoxy” or “aralkoxy,” as used herein, alone or in combination, refers to an aryl group attached to the parent molecular moiety through an alkoxy group.

The term “arylalkyl” or “aralkyl,” as used herein, alone or in combination, refers to an aryl group attached to the parent molecular moiety through an alkyl group.

The term “arylalkynyl” or “aralkynyl,” as used herein, alone or in combination, refers to an aryl group attached to the parent molecular moiety through an alkynyl group.

The term “arylalkanoyl” or “aralkanoyl” or “aroyl,” as used herein, alone or in combination, refers to an acyl radical derived from an aryl-substituted alkanecarboxylic acid such as benzoyl, napthoyl, phenylacetyl, 3-phenylpropionyl (hydrocinnamoyl), 4-phenylbutyryl, (2-naphthyl)acetyl, 4-chlorohydrocinnamoyl, and the like.

The term aryloxy as used herein, alone or in combination, refers to an aryl group attached to the parent molecular moiety through an oxy.

The terms “benzo” and “benz,” as used herein, alone or in combination, refer to the divalent radical C₆H₄═ derived from benzene. Examples include benzothiophene and benzimidazole.

The term “carbamate,” as used herein, alone or in combination, refers to an ester of carbamic acid (—NHCOO—) which may be attached to the parent molecular moiety from either the nitrogen or acid end, and which may be optionally substituted as defined herein.

The term “O-carbamyl” as used herein, alone or in combination, refers to a —OC(O)NRR′, group-with R and R′ as defined herein.

The term “N-carbamyl” as used herein, alone or in combination, refers to a ROC(O)NR′— group, with R and R′ as defined herein.

The term “carbonyl,” as used herein, when alone includes formyl [—C(O)H] and in combination is a —C(O)— group.

The term “carboxyl” or “carboxy,” as used herein, refers to —C(O)OH or the corresponding “carboxylate” anion, such as is in a carboxylic acid salt. An “O-carboxy” group refers to a RC(O)O— group, where R is as defined herein. A “C-carboxy” group refers to a —C(O)OR groups where R is as defined herein.

The term “cyano,” as used herein, alone or in combination, refers to —CN.

The term “cycloalkyl,” or, alternatively, “carbocycle,” as used herein, alone or in combination, refers to a saturated or partially saturated monocyclic, bicyclic or tricyclic alkyl group wherein each cyclic moiety contains from 3 to 12 carbon atom ring members and which may optionally be a benzo fused ring system which is optionally substituted as defined herein. In certain embodiments, said cycloalkyl will comprise from 5 to 7 carbon atoms. Examples of such cycloalkyl groups include cyclopropyl, cyclobutyl, cyclopentyl, cyclohexyl, cycloheptyl, tetrahydronapthyl, indanyl, octahydronaphthyl, 2,3-dihydro-1H-indenyl, adamantyl and the like. “Bicyclic” and “tricyclic” as used herein are intended to include both fused ring systems, such as decahydronaphthalene, octahydronaphthalene as well as the multicyclic (multicentered) saturated or partially unsaturated type. The latter type of isomer is exemplified in general by, bicyclo[1,1,1]pentane, camphor, adamantane, and bicyclo[3,2,1]octane.

The term “ester,” as used herein, alone or in combination, refers to a carboxy group bridging two moieties linked at carbon atoms.

The term “ether,” as used herein, alone or in combination, refers to an oxy group bridging two moieties linked at carbon atoms.

The term “halo,” or “halogen,” as used herein, alone or in combination, refers to fluorine, chlorine, bromine, or iodine.

The term “haloalkoxy,” as used herein, alone or in combination, refers to a haloalkyl group attached to the parent molecular moiety through an oxygen atom.

The term “haloalkyl,” as used herein, alone or in combination, refers to an alkyl radical having the meaning as defined above wherein one or more hydrogens are replaced with a halogen. Specifically embraced are monohaloalkyl, dihaloalkyl and polyhaloalkyl radicals. A monohaloalkyl radical, for one example, may have an iodo, bromo, chloro or fluoro atom within the radical. Dihalo and polyhaloalkyl radicals may have two or more of the same halo atoms or a combination of different halo radicals. Examples of haloalkyl radicals include fluoromethyl, difluoromethyl, trifluoromethyl, chloromethyl, dichloromethyl, trichloromethyl, pentafluoroethyl, heptafluoropropyl, difluorochloromethyl, dichlorofluoromethyl, difluoroethyl, difluoropropyl, dichloroethyl and dichloropropyl. “Haloalkylene” refers to a haloalkyl group attached at two or more positions. Examples include fluoromethylene (—CFH—), difluoromethylene (—CF₂—), chloromethylene (—CHCl—) and the like.

The term “heteroalkyl,” as used herein, alone or in combination, refers to a stable straight or branched chain, or combinations thereof, fully saturated or containing from 1 to 3 degrees of unsaturation, consisting of the stated number of carbon atoms and from one to three heteroatoms chosen from N, O, and S, and wherein the N and S atoms may optionally be oxidized and the N heteroatom may optionally be quaternized. The heteroatom(s) may be placed at any interior position of the heteroalkyl group. Up to two heteroatoms may be consecutive, such as, for example, —CH₂—NH—OCH₃.

The term “heteroaryl,” as used herein, alone or in combination, refers to a 3 to 15 membered unsaturated heteromonocyclic ring, or a fused monocyclic, bicyclic, or tricyclic ring system in which at least one of the fused rings is aromatic, which contains at least one atom chosen from N, O, and S. In certain embodiments, said heteroaryl will comprise from 1 to 4 heteroatoms as ring members. In further embodiments, said heteroaryl will comprise from 1 to 2 heteroatoms as ring members. In certain embodiments, said heteroaryl will comprise from 5 to 7 atoms. The term also embraces fused polycyclic groups wherein heterocyclic rings are fused with aryl rings, wherein heteroaryl rings are fused with other heteroaryl rings, wherein heteroaryl rings are fused with heterocycloalkyl rings, or wherein heteroaryl rings are fused with cycloalkyl rings. Examples of heteroaryl groups include pyrrolyl, pyrrolinyl, imidazolyl, pyrazolyl, pyridyl, pyrimidinyl, pyrazinyl, pyridazinyl, triazolyl, pyranyl, furyl, thienyl, oxazolyl, isoxazolyl, oxadiazolyl, thiazolyl, thiadiazolyl, isothiazolyl, indolyl, isoindolyl, indolizinyl, benzimidazolyl, quinolyl, isoquinolyl, quinoxalinyl, quinazolinyl, indazolyl, benzotriazolyl, benzodioxolyl, benzopyranyl, benzoxazolyl, benzoxadiazolyl, benzothiazolyl, benzothiadiazolyl, benzofuryl, benzothienyl, chromonyl, coumarinyl, benzopyranyl, tetrahydroquinolinyl, tetrazolopyridazinyl, tetrahydroisoquinolinyl, thienopyridinyl, furopyridinyl, pyrrolopyridinyl and the like. Exemplary tricyclic heterocyclic groups include carbazolyl, benzidolyl, phenanthrolinyl, dibenzofuranyl, acridinyl, phenanthridinyl, xanthenyl and the like.

The terms “heterocycloalkyl” and, interchangeably, “heterocycle,” as used herein, alone or in combination, each refer to a saturated, partially unsaturated, or fully unsaturated (but nonaromatic) monocyclic, bicyclic, or tricyclic heterocyclic group containing at least one heteroatom as a ring member, wherein each said heteroatom may be independently chosen from nitrogen, oxygen, and sulfur. In certain embodiments, said hetercycloalkyl will comprise from 1 to 4 heteroatoms as ring members. In further embodiments, said hetercycloalkyl will comprise from 1 to 2 heteroatoms as ring members. In certain embodiments, said hetercycloalkyl will comprise from 3 to 8 ring members in each ring. In further embodiments, said hetercycloalkyl will comprise from 3 to 7 ring members in each ring. In yet further embodiments, said hetercycloalkyl will comprise from 5 to 6 ring members in each ring. “Heterocycloalkyl” and “heterocycle” are intended to include sulfones, sulfoxides, N-oxides of tertiary nitrogen ring members, and carbocyclic fused and benzo fused ring systems; additionally, both terms also include systems where a heterocycle ring is fused to an aryl group, as defined herein, or an additional heterocycle group. Examples of heterocycle groups include aziridinyl, azetidinyl, 1,3-benzodioxolyl, dihydroisoindolyl, dihydroisoquinolinyl, dihydrocinnolinyl, dihydrobenzodioxinyl, dihydro[1,3]oxazolo[4,5-b]pyridinyl, benzothiazolyl, dihydroindolyl, dihy-dropyridinyl, 1,3-dioxanyl, 1,4-dioxanyl, 1,3-dioxolanyl, isoindolinyl, morpholinyl, piperazinyl, pyrrolidinyl, tetrahydropyridinyl, piperidinyl, thiomorpholinyl, and the like. The heterocycle groups may be optionally substituted unless specifically prohibited.

The term “hydrazinyl” as used herein, alone or in combination, refers to two amino groups joined by a single bond, i.e., —N—N—.

The term “hydroxy,” as used herein, alone or in combination, refers to —OH.

The term “hydroxyalkyl,” as used herein, alone or in combination, refers to a hydroxy group attached to the parent molecular moiety through an alkyl group.

The term “imino,” as used herein, alone or in combination, refers to ═N—.

The term “iminohydroxy,” as used herein, alone or in combination, refers to ═N(OH) and ═N—O—.

The phrase “in the main chain” refers to the longest contiguous or adjacent chain of carbon atoms starting at the point of attachment of a group to the compounds of any one of the formulas disclosed herein.

The term “isocyanato” refers to a —NCO group.

The term “isothiocyanato” refers to a —NCS group.

The phrase “linear chain of atoms” refers to the longest straight chain of atoms independently chosen from carbon, nitrogen, oxygen and sulfur.

The term “lower,” as used herein, alone or in a combination, where not otherwise specifically defined, means containing from 1 to and including 6 carbon atoms (i.e., C₁-C₆ alkyl).

The term “lower aryl,” as used herein, alone or in combination, means phenyl or naphthyl, either of which may be optionally substituted as provided.

The term “lower heteroaryl,” as used herein, alone or in combination, means either 1) monocyclic heteroaryl comprising five or six ring members, of which between one and four said members may be heteroatoms chosen from N, O, and S, or 2) bicyclic heteroaryl, wherein each of the fused rings comprises five or six ring members, comprising between them one to four heteroatoms chosen from N, O, and S.

The term “lower cycloalkyl,” as used herein, alone or in combination, means a monocyclic cycloalkyl having between three and six ring members (i.e., C₃-C₆ cycloalkyl). Lower cycloalkyls may be unsaturated. Examples of lower cycloalkyl include cyclopropyl, cyclobutyl, cyclopentyl, and cyclohexyl.

The term “lower heterocycloalkyl,” as used herein, alone or in combination, means a monocyclic heterocycloalkyl having between three and six ring members, of which between one and four may be heteroatoms chosen from N, O, and S (i.e., C₃-C₆ heterocycloalkyl). Examples of lower heterocycloalkyls include pyrrolidinyl, imidazolidinyl, pyrazolidinyl, piperidinyl, piperazinyl, and morpholinyl. Lower heterocycloalkyls may be unsaturated.

The term “lower amino,” as used herein, alone or in combination, refers to —NRR′, wherein R and R′ are independently chosen from hydrogen and lower alkyl, either of which may be optionally substituted.

The term “mercaptyl” as used herein, alone or in combination, refers to an RS— group, where R is as defined herein.

The term “nitro,” as used herein, alone or in combination, refers to —NO₂.

The terms “oxy” or “oxa,” as used herein, alone or in combination, refer to —O—.

The term “oxo,” as used herein, alone or in combination, refers to ═O.

The term “perhaloalkoxy” refers to an alkoxy group where all of the hydrogen atoms are replaced by halogen atoms.

The term “perhaloalkyl” as used herein, alone or in combination, refers to an alkyl group where all of the hydrogen atoms are replaced by halogen atoms.

The terms “sulfonate,” “sulfonic acid,” and “sulfonic,” as used herein, alone or in combination, refer the —SO₃H group and its anion as the sulfonic acid is used in salt formation.

The term “sulfanyl,” as used herein, alone or in combination, refers to —S—.

The term “sulfinyl,” as used herein, alone or in combination, refers to —S(O)—.

The term “sulfonyl,” as used herein, alone or in combination, refers to —S(O)₂—.

The term “N-sulfonamido” refers to a RS(═O)₂NR′— group with R and R′ as defined herein.

The term “S-sulfonamido” refers to a —S(═O)₂NRR′, group, with R and R′ as defined herein.

The terms “thia” and “thio,” as used herein, alone or in combination, refer to a —S— group or an ether wherein the oxygen is replaced with sulfur. The oxidized derivatives of the thio group, namely sulfinyl and sulfonyl, are included in the definition of thia and thio.

The term “thiol,” as used herein, alone or in combination, refers to an —SH group.

The term “thiocarbonyl,” as used herein, when alone includes thioformyl —C(S)H and in combination is a —C(S)— group.

The term “N-thiocarbamyl” refers to an ROC(S)NR′— group, with R and R′ as defined herein.

The term “O-thiocarbamyl” refers to a —OC(S)NRR′, group with R and R′ as defined herein.

The term “thiocyanato” refers to a —CNS group.

The term “trihalomethanesulfonamido” refers to a X₃CS(O)₂NR— group with X is a halogen and R as defined herein.

The term “trihalomethanesulfonyl” refers to a X₃CS(O)₂— group where X is a halogen.

The term “trihalomethoxy” refers to a X₃CO— group where X is a halogen.

The term “trisubstituted silyl,” as used herein, alone or in combination, refers to a silicone group substituted at its three free valences with groups as listed herein under the definition of substituted amino. Examples include trimethysilyl, tert-butyldimethylsilyl, triphenylsilyl and the like.

Any definition herein may be used in combination with any other definition to describe a composite structural group. By convention, the trailing element of any such definition is that which attaches to the parent moiety. For example, the composite group alkylamido would represent an alkyl group attached to the parent molecule through an amido group, and the term alkoxyalkyl would represent an alkoxy group attached to the parent molecule through an alkyl group.

When a group is defined to be “null,” what is meant is that said group is absent.

The term “optionally substituted” means the anteceding group may be substituted or unsubstituted. When substituted, the substituents of an “optionally substituted” group may include, without limitation, one or more substituents independently chosen from the following groups or a particular designated set of groups, alone or in combination: lower alkyl, lower alkenyl, lower alkynyl, lower alkanoyl, lower heteroalkyl, lower heterocycloalkyl, lower haloalkyl, lower haloalkenyl, lower haloalkynyl, lower perhaloalkyl, lower perhaloalkoxy, lower cycloalkyl, phenyl, aryl, aryloxy, lower alkoxy, lower haloalkoxy, oxo, lower acyloxy, carbonyl, carboxyl, lower alkylcarbonyl, lower carboxyester, lower carboxamido, cyano, hydrogen, halogen, hydroxy, amino, lower alkylamino, arylamino, amido, nitro, thiol, lower alkylthio, lower haloalkylthio, lower perhaloalkylthio, arylthio, sulfonate, sulfonic acid, trisubstituted silyl, N₃, SH, SCH₃, C(O)CH₃, CO₂CH₃, CO₂H, pyridinyl, thiophene, furanyl, lower carbamate, and lower urea. Where structurally feasible, two substituents may be joined together to form a fused five-, six-, or seven-membered carbocyclic or heterocyclic ring consisting of zero to three heteroatoms, for example forming methylenedioxy or ethylenedioxy. An optionally substituted group may be unsubstituted (e.g., —CH₂CH₃), fully substituted (e.g., —CF₂CF₃), monosubstituted (e.g., —CH₂CH₂F) or substituted at a level anywhere in-between fully substituted and monosubstituted (e.g., —CH₂CF₃). Where substituents are recited without qualification as to substitution, both substituted and unsubstituted forms are encompassed. Where a substituent is qualified as “substituted,” the substituted form is specifically intended. Additionally, different sets of optional substituents to a particular moiety may be defined as needed; in these cases, the optional substitution will be as defined, often immediately following the phrase, “optionally substituted with.”

The term R or the term R′, appearing by itself and without a number designation, unless otherwise defined, refers to a moiety chosen from hydrogen, alkyl, cycloalkyl, heteroalkyl, aryl, heteroaryl and heterocycloalkyl, any of which may be optionally substituted. Such R and R′ groups should be understood to be optionally substituted as defined herein. Whether an R group has a number designation or not, every R group, including R, R′ and Rⁿ where n=(1, 2, 3, . . . n), every substituent, and every term should be understood to be independent of every other in terms of selection from a group. Should any variable, substituent, or term (e.g. aryl, heterocycle, R, etc.) occur more than one time in a formula or generic structure, its definition at each occurrence is independent of the definition at every other occurrence. Those of skill in the art will further recognize that certain groups may be attached to a parent molecule or may occupy a position in a chain of elements from either end as written. For example, an unsymmetrical group such as —C(O)N(R)— may be attached to the parent moiety at either the carbon or the nitrogen.

Asymmetric centers exist in the compounds disclosed herein. These centers are designated by the symbols “R” or “S,” depending on the configuration of substituents around the chiral carbon atom. It should be understood that the disclosure encompasses all stereochemical isomeric forms, including diastereomeric, enantiomeric, and epimeric forms, as well as d-isomers and 1-isomers, and mixtures thereof. Individual stereoisomers of compounds can be prepared synthetically from commercially available starting materials which contain chiral centers or by preparation of mixtures of enantiomeric products followed by separation such as conversion to a mixture of diastereomers followed by separation or recrystallization, chromatographic techniques, direct separation of enantiomers on chiral chromatographic columns, or any other appropriate method known in the art. Starting compounds of particular stereochemistry are either commercially available or can be made and resolved by techniques known in the art. Additionally, the compounds disclosed herein may exist as geometric isomers. The present disclosure includes all cis, trans, syn, anti, entgegen (E), and zusammen (Z) isomers as well as the appropriate mixtures thereof. Additionally, compounds may exist as tautomers; all tautomeric isomers are provided by this disclosure. Additionally, the compounds disclosed herein can exist in unsolvated as well as solvated forms with pharmaceutically acceptable solvents such as water, ethanol, and the like. In general, the solvated forms are considered equivalent to the unsolvated forms.

The term “bond” refers to a covalent linkage between two atoms, or two moieties when the atoms joined by the bond are considered to be part of larger substructure. A bond may be single, double, or triple unless otherwise specified. A dashed line between two atoms in a drawing of a molecule indicates that an additional bond may be present or absent at that position.

The compounds disclosed herein can exist as therapeutically acceptable salts. The present disclosure includes compounds listed above in the form of salts, including acid addition salts. Suitable salts include those formed with both organic and inorganic acids. Such acid addition salts will normally be pharmaceutically acceptable. However, salts of non-pharmaceutically acceptable salts may be of utility in the preparation and purification of the compound in question. Basic addition salts may also be formed and be pharmaceutically acceptable. For a more complete discussion of the preparation and selection of salts, refer to Pharmaceutical Salts: Properties, Selection, and Use (Stahl, P. Heinrich. Wiley-VCHA, Zurich, Switzerland, 2002).

The term “therapeutically acceptable salt,” as used herein, represents salts or zwitterionic forms of the compounds disclosed herein which are water or oil-soluble or dispersible and therapeutically acceptable as defined herein. The salts can be prepared during the final isolation and purification of the compounds or separately by reacting the appropriate compound in the form of the free base with a suitable acid. Representative acid addition salts include acetate, adipate, alginate, L-ascorbate, aspartate, benzoate, benzenesulfonate (besylate), bisulfate, butyrate, camphorate, camphorsulfonate, citrate, digluconate, formate, fumarate, gentisate, glutarate, glycerophosphate, glycolate, hemisulfate, heptanoate, hexanoate, hippurate, hydrochloride, hydrobromide, hydroiodide, 2-hydroxyethansulfonate (isethionate), lactate, maleate, malonate, DL-mandelate, mesitylenesulfonate, methanesulfonate, naphthylenesulfonate, nicotinate, 2-naphthalenesulfonate, oxalate, pamoate, pectinate, persulfate, 3-phenylproprionate, phosphonate, picrate, pivalate, propionate, pyroglutamate, succinate, sulfonate, tartrate, L-tartrate, trichloroacetate, trifluoroacetate, phosphate, glutamate, bicarbonate, para-toluenesulfonate (p-tosylate), and undecanoate. Also, basic groups in the compounds disclosed herein can be quaternized with methyl, ethyl, propyl, and butyl chlorides, bromides, and iodides; dimethyl, diethyl, dibutyl, and diamyl sulfates; decyl, lauryl, myristyl, and steryl chlorides, bromides, and iodides; and benzyl and phenethyl bromides. Examples of acids which can be employed to form therapeutically acceptable addition salts include inorganic acids such as hydrochloric, hydrobromic, sulfuric, and phosphoric, and organic acids such as oxalic, maleic, succinic, and citric. Salts can also be formed by coordination of the compounds with an alkali metal or alkaline earth ion. Hence, the present disclosure contemplates sodium, potassium, magnesium, and calcium salts of the compounds disclosed herein, and the like.

Basic addition salts can be prepared during the final isolation and purification of the compounds by reacting a carboxy group with a suitable base such as the hydroxide, carbonate, or bicarbonate of a metal cation or with ammonia or an organic primary, secondary, or tertiary amine. The cations of therapeutically acceptable salts include lithium, sodium, potassium, calcium, magnesium, and aluminum, as well as nontoxic quaternary amine cations such as ammonium, tetramethylammonium, tetraethylammonium, methylamine, dimethylamine, trimethylamine, triethylamine, diethylamine, ethylamine, tributylamine, pyridine, N,N-dimethylaniline, N-methylpiperidine, N-methylmorpholine, dicyclohexylamine, procaine, dibenzylamine, N,N-dibenzylphenethylamine, 1-ephenamine, and N,N-dibenzylethylenediamine. Other representative organic amines useful for the formation of base addition salts include ethylenediamine, ethanolamine, diethanolamine, piperidine, and piperazine.

While it may be possible for the compounds of the subject disclosure to be administered as the raw chemical, it is also possible to present them as a pharmaceutical formulation. Accordingly, provided herein are pharmaceutical formulations which comprise one or more of certain compounds disclosed herein, or one or more pharmaceutically acceptable salts, esters, prodrugs, amides, or solvates thereof, together with one or more pharmaceutically acceptable carriers thereof and optionally one or more other therapeutic ingredients. The carrier(s) must be “acceptable” in the sense of being compatible with the other ingredients of the formulation and not deleterious to the recipient thereof. Proper formulation is dependent upon the route of administration chosen. Any of the well-known techniques, carriers, and excipients may be used as suitable and as understood in the art. The pharmaceutical compositions disclosed herein may be manufactured in any manner known in the art, e.g., by means of conventional mixing, dissolving, granulating, dragee-making, levigating, emulsifying, encapsulating, entrapping or compression processes.

The formulations include those suitable for oral, parenteral (including subcutaneous, intradermal, intramuscular, intravenous, intraarticular, and intramedullary), intraperitoneal, transmucosal, transdermal, rectal and topical (including dermal, buccal, sublingual and intraocular) administration although the most suitable route may depend upon for example the condition and disorder of the recipient. The formulations may conveniently be presented in unit dosage form and may be prepared by any of the methods well known in the art of pharmacy. Typically, these methods include the step of bringing into association a compound of the subject disclosure or a pharmaceutically acceptable salt, ester, amide, prodrug or solvate thereof (“active ingredient”) with the carrier which constitutes one or more accessory ingredients. In general, the formulations are prepared by uniformly and intimately bringing into association the active ingredient with liquid carriers or finely divided solid carriers or both and then, if necessary, shaping the product into the desired formulation.

### II. Anti-HER2/HER3 Antibodies

An “anti-HER2/HER3 antibody” as used herein includes any molecule that interferes with the function of HER2 and/or HER3. Thus, an anti-HER2/HER3 antibody includes an anti-HER2 antibody (e.g., trastuzumab or pertuzumab), an anti-HER3 antibody, and an anti-HER2/HER3 bispecific antibody (e.g., the antibodies disclosed in WO2018/182422 or MCLA-128). A HER2/HER3 targeting antibody may prevent the formation of HER2/HER2 dimers and/or HER2/HER3 dimers (e.g., trastuzumab or pertuzumab). In some cases, a HER2/HER3 targeting antibody may be an antibody drug conjugate (e.g., T-DM1 or U3-1402).

In certain embodiments, the HER2/HER3 targeting antibody is trastuzumab (Genentech and Roche), trastuzumab emtansine (T-DM1; Genentech and Roche), pertuzumab (Genentech), ertumaxomab (Fresenius), margetuximab (MacroGenics), MCLA-128 (zenocutuzumab; Merus) MM-111 (Merrimack), MM-121 (Merrimack), CT-P06 (Celltrion), GSK2849330 (GlaxoSmithKline), PF-05280014 (Pfizer), MM-302 (Merrimack), SB3 (Merck & Co), CMAB302 (Shanghai CP Guojian), RG7116 (lemretuzumab; Genentech/Roche), TrasGEX (Glycotope), ARX788 (Ambrx and Zhejiang Medicine), SYD985 (Synthon), FS102 (Bristol-Myers Squibb and f-star), BCD-022 (Biocad), ABP 980 (Amgen), DS-8201a (Daiichi Sankyo), HLX02 (Shanghai Henlius), SAR256212 (Sanofi Oncology), RG7597 (Genentech), U3-1402 (Daiichi Sankyo), or CANMAb (Biocon and Mylan).

Trastuzumab (CAS 180288-69-1, HERCEPTIN®, huMAb4D5-8, rhuMAb HER2, Genentech) is a humanized, IgG1 kappa, monoclonal antibody that selectively binds with high affinity to the extracellular domain of the human epidermal growth factor receptor 2 protein, HER2 (ErbB2) (U.S. Pat. Nos. 5,677,171; 5,821,337; 6,054,297; 6,165,464; 6,339,142; 6,407,213; 6,639,055; 6,719,971; 6,800,738; 7,074,404). Trastuzumab contains human framework regions with the complementarity-determining regions of a murine antibody (4D5) that binds to HER2. Trastuzumab binds to the HER2 antigen and thus inhibits the growth of cancerous cells. Trastuzumab has been shown, in both in vitro assays and in animals, to inhibit the proliferation of human tumor cells that overexpress HER2. Trastuzumab is a mediator of antibody-dependent cellular cytotoxicity, ADCC.

Trastuzumab emtansine, also known as ado-trastuzumab emtansine and sold under the trade name KADCYLA®, is an antibody-drug conjugate consisting of the humanized monoclonal antibody trastuzumab covalently linked to the cytotoxic agent emtansine (DM1). Trastuzumab alone stops growth of cancer cells by binding to the HER2 receptor, whereas trastuzumab emtansine undergoes receptor-mediated internalization into cells, is catabolized in lysosomes where DM1-containing catabolites are released and subsequently bind tubulin to cause mitotic arrest and cell death. Trastuzumab binding to HER2 prevents homodimerization or heterodimerization (HER2/HER3) of the receptor, ultimately inhibiting the activation of MAPK and PI3K/AKT cellular signaling pathways. Because the monoclonal antibody targets HER2, and HER2 is only over-expressed in cancer cells, the conjugate delivers the cytotoxic agent DM1 specifically to tumor cells. The conjugate is abbreviated T-DM1. T-DM1 may be administered at a dose of 2-3 mg/kg, such as 3.6 mg/kg. The T-DM1 may be administered by intravenous infusion.

Pertuzumab (CAS Reg. No. 380610-27-5, OMNITARG®, 2C4, Genentech) is a recombinant, humanized monoclonal antibody that inhibits dimerization of HER2 (U.S. Pat. Nos. 6,054,297; 6,407,213; 6,800,738; 6,627,196, 6,949,245; 7,041,292). Pertuzumab contains the human IgG1 (x) framework sequences. Pertuzumab and trastuzumab target different extracellular regions of the HER2 tyrosine kinase receptor. Pertuzumab binds to an epitope within sub-domain 2 of HER2, while the epitope from trastuzumab is localized to sub-domain 4. Pertuzumab blocks the ability of the HER2 receptor to collaborate with other HER receptor family members, i.e., HER1/EGFR, HER3, and HER4 (U.S. Pat. No. 6,949,245). In cancer cells, interfering with the ability of HER2 to collaborate with other HER family receptors blocks cell signaling and may ultimately lead to cancer cell growth inhibition and death of the cancer cell.

Additional exemplary anti-HER2/IER3 antibodies include MM-121/SAR256212, which is a fully human monoclonal antibody that targets the HER3 receptor and which has been reported to be useful in the treatment of non-small cell lung cancer (NSCLC), breast cancer and ovarian cancer. SAR256212 is an investigational fully human monoclonal antibody that targets the HER3 (ErbB3) receptor. Duligotuzmab (MEHD7945A, RG7597) is a humanized IgG1 monoclonal antibody that targets HER1 and HER3, and has been described as being useful in head and neck cancers. Margetuximab (MGAH22) is an Fc-optimized monoclonal antibody that targets HER2.

Antibodies according to the present disclosure may be defined, in the first instance, by their binding specificity. Those of skill in the art, by assessing the binding specificity/affinity of a given antibody using techniques well known to those of skill in the art, can determine whether such antibodies fall within the scope of the instant claims. Various techniques known to persons of ordinary skill in the art can be used to determine whether an antibody interacts with a polypeptide or protein. Exemplary techniques include, for example, routine cross-blocking assays. Cross-blocking can be measured in various binding assays such as ELISA, biolayer interferometry, or surface plasmon resonance. Other methods include alanine scanning mutational analysis, peptide blot analysis, peptide cleavage analysis, high-resolution electron microscopy techniques using single particle reconstruction, cryoEM, or tomography, crystallographic studies, and NMR analysis.

The present disclosure includes antibodies that may bind to the same epitope, or a portion of the epitope. Likewise, the present disclosure also includes antibodies that compete for binding to a target or a fragment thereof with any of the specific exemplary antibodies described herein. One can easily determine whether an antibody binds to the same epitope as, or competes for binding with, a reference antibody by using routine methods known in the art. For example, to determine if a test antibody binds to the same epitope as a reference, the reference antibody is allowed to bind to target under saturating conditions. Next, the ability of a test antibody to bind to the target molecule is assessed. If the test antibody is able to bind to the target molecule following saturation binding with the reference antibody, it can be concluded that the test antibody binds to a different epitope than the reference antibody. On the other hand, if the test antibody is not able to bind to the target molecule following saturation binding with the reference antibody, then the test antibody may bind to the same epitope as the epitope bound by the reference antibody.

Two antibodies bind to the same or overlapping epitope if each competitively inhibits (blocks) binding of the other to the antigen. That is, a 1-, 5-, 10-, 20- or 100-fold excess of one antibody inhibits binding of the other by at least 50% but preferably 75%, 90% or even 99% as measured in a competitive binding assay. Alternatively, two antibodies have the same epitope if essentially all amino acid mutations in the antigen that reduce or eliminate binding of one antibody reduce or eliminate binding of the other. Two antibodies have overlapping epitopes if some amino acid mutations that reduce or eliminate binding of one antibody reduce or eliminate binding of the other.

Additional routine experimentation (e.g., peptide mutation and binding analyses) can then be carried out to confirm whether the observed lack of binding of the test antibody is in fact due to binding to the same epitope as the reference antibody or if steric blocking (or another phenomenon) is responsible for the lack of observed binding. Experiments of this sort can be performed using ELISA, RIA, surface plasmon resonance, flow cytometry or any other quantitative or qualitative antibody-binding assay available in the art. Structural studies with EM or crystallography also can demonstrate whether or not two antibodies that compete for binding recognize the same epitope.

In another aspect, the antibodies may be defined by their variable sequence, which include additional “framework” regions. Furthermore, the antibodies sequences may vary from these sequences, optionally using methods discussed in greater detail below. For example, nucleic acid sequences may vary from those set out above in that (a) the variable regions may be segregated away from the constant domains of the light and heavy chains, (b) the nucleic acids may vary from those set out above while not affecting the residues encoded thereby, (c) the nucleic acids may vary from those set out above by a given percentage, e.g., 70%, 75%, 80%, 85%, 90%, 91%, 92%, 93%, 94%, 95%, 96%, 97%, 98% or 99% homology, (d) the nucleic acids may vary from those set out above by virtue of the ability to hybridize under high stringency conditions, as exemplified by low salt and/or high temperature conditions, such as provided by about 0.02 M to about 0.15 M NaCl at temperatures of about 50° C. to about 70° C., (e) the amino acids may vary from those set out above by a given percentage, e.g., 80%, 85%, 90%, 91%, 92%, 93%, 94%, 95%, 96%, 97%, 98% or 99% homology, or (f) the amino acids may vary from those set out above by permitting conservative substitutions (discussed below).

When comparing polynucleotide and polypeptide sequences, two sequences are said to be “identical” if the sequence of nucleotides or amino acids in the two sequences is the same when aligned for maximum correspondence, as described below. Comparisons between two sequences are typically performed by comparing the sequences over a comparison window to identify and compare local regions of sequence similarity. A “comparison window” as used herein, refers to a segment of at least about 20 contiguous positions, usually 30 to about 75, 40 to about 50, in which a sequence may be compared to a reference sequence of the same number of contiguous positions after the two sequences are optimally aligned.

Optimal alignment of sequences for comparison may be conducted using the Megalign program in the Lasergene suite of bioinformatics software (DNASTAR, Inc., Madison, Wis.), using default parameters. This program embodies several alignment schemes described in the following references: Dayhoff, M. O. (1978) A model of evolutionary change in proteins—Matrices for detecting distant relationships. In Dayhoff, M. O. (ed.) Atlas of Protein Sequence and Structure, National Biomedical Research Foundation, Washington D.C. Vol. 5, Suppl. 3, pp. 345-358; Hein J. (1990) Unified Approach to Alignment and Phylogeny pp. 626-645 Methods in Enzymology vol. 183, Academic Press, Inc., San Diego, Calif.; Higgins, D. G. and Sharp, P. M. (1989) CABIOS 5:151-153; Myers, E. W. and Muller W. (1988) CABIOS 4:11-17; Robinson, E. D. (1971) Comb. Theor 11:105; Santou, N. Nes, M. (1987) Mol. Biol. Evol. 4:406-425; Sneath, P. H. A. and Sokal, R. R. (1973) Numerical Taxonomy—the Principles and Practice of Numerical Taxonomy, Freeman Press, San Francisco, Calif.; Wilbur, W. J. and Lipman, D. J. (1983) Proc. Natl. Acad., Sci. USA 80:726-730.

Alternatively, optimal alignment of sequences for comparison may be conducted by the local identity algorithm of Smith and Waterman (1981) Add. APL. Math 2:482, by the identity alignment algorithm of Needleman and Wunsch (1970) J. Mol. Biol. 48:443, by the search for similarity methods of Pearson and Lipman (1988) Proc. Natl. Acad. Sci. USA 85: 2444, by computerized implementations of these algorithms (GAP, BESTFIT, BLAST, FASTA, and TFASTA in the Wisconsin Genetics Software Package, Genetics Computer Group (GCG), 575 Science Dr., Madison, Wis.), or by inspection.

One particular example of algorithms that are suitable for determining percent sequence identity and sequence similarity are the BLAST and BLAST 2.0 algorithms, which are described in Altschul et al. (1977) Nucl. Acids Res. 25:3389-3402 and Altschul et al. (1990) J. Mol. Biol. 215:403-410, respectively. BLAST and BLAST 2.0 can be used, for example with the parameters described herein, to determine percent sequence identity for the polynucleotides and polypeptides of the disclosure. Software for performing BLAST analyses is publicly available through the National Center for Biotechnology Information. The rearranged nature of an antibody sequence and the variable length of each gene requires multiple rounds of BLAST searches for a single antibody sequence. Also, manual assembly of different genes is difficult and error-prone. The sequence analysis tool IgBLAST (world-wide-web at ncbi.nlm.nih.gov/igblast/) identifies matches to the germline V, D and J genes, details at rearrangement junctions, the delineation of Ig V domain framework regions and complementarity determining regions. IgBLAST can analyze nucleotide or protein sequences and can process sequences in batches and allows searches against the germline gene databases and other sequence databases simultaneously to minimize the chance of missing possibly the best matching germline V gene.

In one illustrative example, cumulative scores can be calculated using, for nucleotide sequences, the parameters M (reward score for a pair of matching residues; always >0) and N (penalty score for mismatching residues; always <0). Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T and X determine the sensitivity and speed of the alignment. The BLASTN program (for nucleotide sequences) uses as defaults a wordlength (W) of 11, and expectation (E) of 10, and the BLOSUM62 scoring matrix (see Henikoff and Henikoff (1989) Proc. Natl. Acad. Sci. USA 89:10915) alignments, (B) of 50, expectation (E) of 10, M=5, N=−4 and a comparison of both strands.

For amino acid sequences, a scoring matrix can be used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T and X determine the sensitivity and speed of the alignment.

In one approach, the “percentage of sequence identity” is determined by comparing two optimally aligned sequences over a window of comparison of at least 20 positions, wherein the portion of the polynucleotide or polypeptide sequence in the comparison window may comprise additions or deletions (i.e., gaps) of 20 percent or less, usually 5 to 15 percent, or 10 to 12 percent, as compared to the reference sequences (which does not comprise additions or deletions) for optimal alignment of the two sequences. The percentage is calculated by determining the number of positions at which the identical nucleic acid bases or amino acid residues occur in both sequences to yield the number of matched positions, dividing the number of matched positions by the total number of positions in the reference sequence (i.e., the window size) and multiplying the results by 100 to yield the percentage of sequence identity.

Yet another way of defining an antibody is as a “derivative” of any of the described antibodies and their antigen-binding fragments. The term “derivative” refers to an antibody or antigen-binding fragment thereof that immunospecifically binds to an antigen but which comprises, one, two, three, four, five or more amino acid substitutions, additions, deletions or modifications relative to a “parental” (or wild-type) molecule. Such amino acid substitutions or additions may introduce naturally occurring (i.e., DNA-encoded) or non-naturally occurring amino acid residues. The term “derivative” encompasses, for example, as variants having altered CH1, hinge, CH2, CH3 or CH4 regions, so as to form, for example antibodies, etc., having variant Fc regions that exhibit enhanced or impaired effector or binding characteristics. The term “derivative” additionally encompasses non-amino acid modifications, for example, amino acids that may be glycosylated (e.g., have altered mannose, 2-N-acetylglucosamine, galactose, fucose, glucose, sialic acid, 5-N-acetylneuraminic acid, 5-glycolneuraminic acid, etc. content), acetylated, pegylated, phosphorylated, amidated, derivatized by known protecting/blocking groups, proteolytic cleavage, linked to a cellular ligand or other protein, etc. In some embodiments, the altered carbohydrate modifications modulate one or more of the following: solubilization of the antibody, facilitation of subcellular transport and secretion of the antibody, promotion of antibody assembly, conformational integrity, and antibody-mediated effector function. In a specific embodiment, the altered carbohydrate modifications enhance antibody mediated effector function relative to the antibody lacking the carbohydrate modification. Carbohydrate modifications that lead to altered antibody mediated effector function are well known in the art.

A derivative antibody or antibody fragment can be generated with an engineered sequence or glycosylation state to confer preferred levels of activity in antibody dependent cellular cytotoxicity (ADCC), antibody-dependent cellular phagocytosis (ADCP), antibody-dependent neutrophil phagocytosis (ADNP), or antibody-dependent complement deposition (ADCD) functions as measured by bead-based or cell-based assays or in vivo studies in animal models.

A derivative antibody or antibody fragment may be modified by chemical modifications using techniques known to those of skill in the art, including, but not limited to, specific chemical cleavage, acetylation, formulation, metabolic synthesis of tunicamycin, etc. In one embodiment, an antibody derivative will possess a similar or identical function as the parental antibody. In another embodiment, an antibody derivative will exhibit an altered activity relative to the parental antibody. For example, a derivative antibody (or fragment thereof) can bind to its epitope more tightly or be more resistant to proteolysis than the parental antibody.

### III. Methods of Treatment

The present invention provides methods of treating a cancer patient with a quinazoline-based TKI, either alone or in combination with an anti-HER2/HER3 antibody. Such treatment may also be in combination with another therapeutic regime, such as chemotherapy or immunotherapy. Certain aspects of the present invention can be used to select a cancer patient for treatment based on the presence of an NRG1 fusion in the patient's cancer cells. In various aspects, about 5%, 10%, 15%, 20%, 25%, 30%, 35%, 40%, 45%, 50%, 55%, 60%, 65%, 70%, 75%, 80%, 85%, 90%, 95%, or 100% of the cells that comprise the cancer may harbor an NRG1 fusion, which indicates that the patient is a candidate for treatment. In some aspects, the patient's cancer cells lack a mutation at EGFR T790 and/or at EGFR C797. In some aspects, the patient's cancer cells lack a mutation at HER2 T798 and/or at HER2 C805.

In certain aspects, the subject was determined to have an NRG1 fusion by analyzing a genomic sample from the subject. In some aspects, the genomic sample is isolated from saliva, blood, urine, or tumor tissue. In particular aspects, the presence of an NRG1 fusion is determined by nucleic acid sequencing (e.g., DNA sequencing of tumor tissue or circulating free DNA from plasma) or PCR analyses.

In certain aspects, the quinazoline-based TKI and/or anti-HER2/IER3 antibody are administered intravenously, subcutaneously, intraosseously, orally, transdermally, in sustained release, in controlled release, in delayed release, as a suppository, or sublingually. In some aspects, administering the quinazoline-based TKI and/or anti-HER2/IER3 antibody comprises local, regional or systemic administration. In particular aspects, the quinazoline-based TKI and/or anti-HER2/HER3 antibody are administered two or more times, such as daily, every other day, or weekly. The quinazoline-based TKI and the anti-HER2/HER3 antibody need not be administered by the same route or on the same schedule.

In some aspects, the quinazoline-based TKI is administered prior to or after the anti-HER2/HER3 antibody, such as 1 day, 2 days, 3 days, 4 days, 5 days, 6 days, 7 days, 2 weeks, 3 weeks, 1 month or more apart. In some aspects, the quinazoline-based TKI is administered simultaneously with the anti-HER2/IER3 antibody.

The term “subject” or “patient” as used herein refers to any individual to which the subject methods are performed. Generally the patient is human, although as will be appreciated by those in the art, the patient may be an animal. Thus other animals, including mammals such as rodents (including mice, rats, hamsters and guinea pigs), cats, dogs, rabbits, farm animals including cows, horses, goats, sheep, pigs, etc., and primates (including monkeys, chimpanzees, orangutans and gorillas) are included within the definition of patient.

“Treatment” and “treating” refer to administration or application of a therapeutic agent to a subject or performance of a procedure or modality on a subject for the purpose of obtaining a therapeutic benefit of a disease or health-related condition. For example, a treatment may include administration chemotherapy, immunotherapy, radiotherapy, performance of surgery, or any combination thereof.

The methods described herein are useful in inhibiting survival or proliferation of cells (e.g., tumor cells), treating proliferative disease (e.g., cancer, psoriasis), and treating pathogenic infection. Generally, the terms “cancer” and “cancerous” refer to or describe the physiological condition in mammals that is typically characterized by unregulated cell growth. More specifically, cancers that are treated in connection with the methods provided herein include, but are not limited to, solid tumors, metastatic cancers, or non-metastatic cancers. In certain embodiments, the cancer may originate in the lung, kidney, bladder, blood, bone, bone marrow, brain, breast, colon, esophagus, duodenum, small intestine, large intestine, colon, rectum, anus, gum, head, liver, nasopharynx, neck, ovary, pancreas, prostate, skin, stomach, testis, tongue, or uterus.

The cancer may specifically be of the following histological type, though it is not limited to these: neoplasm, malignant; carcinoma; non-small cell lung cancer; renal cancer; renal cell carcinoma; clear cell renal cell carcinoma; lymphoma; blastoma; sarcoma; carcinoma, undifferentiated; meningioma; brain cancer; oropharyngeal cancer; nasopharyngeal cancer; biliary cancer; pheochromocytoma; pancreatic islet cell cancer; Li-Fraumeni tumor; thyroid cancer; parathyroid cancer; pituitary tumor; adrenal gland tumor; osteogenic sarcoma tumor; neuroendocrine tumor; breast cancer; lung cancer; head and neck cancer; prostate cancer; esophageal cancer; tracheal cancer; liver cancer; bladder cancer; stomach cancer; pancreatic cancer; ovarian cancer; uterine cancer; cervical cancer; testicular cancer; colon cancer; rectal cancer; skin cancer; giant and spindle cell carcinoma; small cell carcinoma; small cell lung cancer; papillary carcinoma; oral cancer; oropharyngeal cancer; nasopharyngeal cancer; respiratory cancer; urogenital cancer; squamous cell carcinoma; lymphoepithelial carcinoma; basal cell carcinoma; pilomatrix carcinoma; transitional cell carcinoma; papillary transitional cell carcinoma; adenocarcinoma; gastrointestinal cancer; gastrinoma, malignant; cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma; carcinoid tumor, malignant; branchiolo-alveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; nonencapsulating sclerosing carcinoma; adrenal cortical carcinoma; endometroid carcinoma; skin appendage carcinoma; apocrine adenocarcinoma; sebaceous adenocarcinoma; ceruminous adenocarcinoma; mucoepidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating duct carcinoma; medullary carcinoma; lobular carcinoma; inflammatory carcinoma; paget's disease, mammary; acinar cell carcinoma; adenosquamous carcinoma; adenocarcinoma with squamous metaplasia; thymoma, malignant; ovarian stromal tumor, malignant; thecoma, malignant; granulosa cell tumor, malignant; androblastoma, malignant; sertoli cell carcinoma; leydig cell tumor, malignant; lipid cell tumor, malignant; paraganglioma, malignant; extra-mammary paraganglioma, malignant; pheochromocytoma; glomangiosarcoma; malignant melanoma; amelanotic melanoma; superficial spreading melanoma; malignant melanoma in giant pigmented nevus; lentigo maligna melanoma; acral lentiginous melanoma; nodular melanoma; epithelioid cell melanoma; blue nevus, malignant; sarcoma; fibrosarcoma; fibrous histiocytoma, malignant; myxosarcoma; liposarcoma; leiomyosarcoma; rhabdomyosarcoma; embryonal rhabdomyosarcoma; alveolar rhabdomyosarcoma; stromal sarcoma; mixed tumor, malignant; mullerian mixed tumor; nephroblastoma; hepatoblastoma; carcinosarcoma; mesenchymoma, malignant; brenner tumor, malignant; phyllodes tumor, malignant; synovial sarcoma; mesothelioma, malignant; dysgerminoma; embryonal carcinoma; teratoma, malignant; struma ovarii, malignant; choriocarcinoma; mesonephroma, malignant; hemangiosarcoma; hemangioendothelioma, malignant; kaposi's sarcoma; hemangiopericytoma, malignant; lymphangiosarcoma; osteosarcoma; juxtacortical osteosarcoma; chondrosarcoma; chondroblastoma, malignant; mesenchymal chondrosarcoma; giant cell tumor of bone; ewing's sarcoma; odontogenic tumor, malignant; ameloblastic odontosarcoma; ameloblastoma, malignant; ameloblastic fibrosarcoma; an endocrine or neuroendocrine cancer or hematopoietic cancer; pinealoma, malignant; chordoma; central or peripheral nervous system tissue cancer; glioma, malignant; ependymoma; astrocytoma; protoplasmic astrocytoma; fibrillary astrocytoma; astroblastoma; glioblastoma; oligodendroglioma; oligodendroblastoma; primitive neuroectodermal; cerebellar sarcoma; ganglioneuroblastoma; neuroblastoma; retinoblastoma; olfactory neurogenic tumor; meningioma, malignant; neurofibrosarcoma; neurilemmoma, malignant; granular cell tumor, malignant; B-cell lymphoma; malignant lymphoma; Hodgkin's disease; Hodgkin's; low grade/follicular non-Hodgkin's lymphoma; paragranuloma; malignant lymphoma, small lymphocytic; malignant lymphoma, large cell, diffuse; malignant lymphoma, follicular; mycosis fungoides; mantle cell lymphoma; Waldenstrom's macroglobulinemia; other specified non-hodgkin's lymphomas; malignant histiocytosis; multiple myeloma; mast cell sarcoma; immunoproliferative small intestinal disease; leukemia; lymphoid leukemia; plasma cell leukemia; erythroleukemia; lymphosarcoma cell leukemia; myeloid leukemia; basophilic leukemia; eosinophilic leukemia; monocytic leukemia; mast cell leukemia; megakaryoblastic leukemia; myeloid sarcoma; chronic lymphocytic leukemia (CLL); acute lymphoblastic leukemia (ALL); Hairy cell leukemia; chronic myeloblastic leukemia; and hairy cell leukemia.

The term “therapeutic benefit” or “therapeutically effective” as used throughout this application refers to anything that promotes or enhances the well-being of the subject with respect to the medical treatment of this condition. This includes, but is not limited to, a reduction in the frequency or severity of the signs or symptoms of a disease. For example, treatment of cancer may involve, for example, a reduction in the invasiveness of a tumor, reduction in the growth rate of the cancer, or prevention of metastasis. Treatment of cancer may also refer to prolonging survival of a subject with cancer.

Likewise, an effective response of a patient or a patient's “responsiveness” to treatment refers to the clinical or therapeutic benefit imparted to a patient at risk for, or suffering from, a disease or disorder. Such benefit may include cellular or biological responses, a complete response, a partial response, a stable disease (without progression or relapse), or a response with a later relapse. For example, an effective response can be reduced tumor size or progression-free survival in a patient diagnosed with cancer.

Regarding neoplastic condition treatment, depending on the stage of the neoplastic condition, neoplastic condition treatment involves one or a combination of the following therapies: surgery to remove the neoplastic tissue, radiation therapy, and chemotherapy. Other therapeutic regimens may be combined with the administration of the anticancer agents, e.g., therapeutic compositions and chemotherapeutic agents. For example, the patient to be treated with such anti-cancer agents may also receive radiation therapy and/or may undergo surgery.

For the treatment of disease, the appropriate dosage of a therapeutic composition will depend on the type of disease to be treated, as defined above, the severity and course of the disease, previous therapy, the patient's clinical history and response to the agent, and the discretion of the physician. The agent may be suitably administered to the patient at one time or over a series of treatments.

The methods and compositions, including combination therapies, enhance the therapeutic or protective effect, and/or increase the therapeutic effect of another anti-cancer or anti-hyperproliferative therapy. Therapeutic and prophylactic methods and compositions can be provided in a combined amount effective to achieve the desired effect, such as the killing of a cancer cell and/or the inhibition of cellular hyperproliferation. A tissue, tumor, or cell can be contacted with one or more compositions or pharmacological formulation(s) comprising one or more of the agents or by contacting the tissue, tumor, and/or cell with two or more distinct compositions or formulations. Also, it is contemplated that such a combination therapy can be used in conjunction with radiotherapy, surgical therapy, or immunotherapy.

Administration in combination can include simultaneous administration of two or more agents in the same dosage form, simultaneous administration in separate dosage forms, and separate administration. That is, the subject therapeutic composition and another therapeutic agent can be formulated together in the same dosage form and administered simultaneously. Alternatively, subject therapeutic composition and another therapeutic agent can be simultaneously administered, wherein both the agents are present in separate formulations. In another alternative, the therapeutic agent can be administered just followed by the other therapeutic agent or vice versa. In the separate administration protocol, the subject therapeutic composition and another therapeutic agent may be administered a few minutes apart, or a few hours apart, or a few days apart.

An anti-cancer first treatment may be administered before, during, after, or in various combinations relative to a second anti-cancer treatment. The administrations may be in intervals ranging from concurrently to minutes to days to weeks. In embodiments where the first treatment is provided to a patient separately from the second treatment, one would generally ensure that a significant period of time did not expire between the time of each delivery, such that the two compounds would still be able to exert an advantageously combined effect on the patient. In such instances, it is contemplated that one may provide a patient with the first therapy and the second therapy within about 12 to 24 or 72 h of each other and, more particularly, within about 6-12 h of each other. In some situations, it may be desirable to extend the time period for treatment significantly where several days (2, 3, 4, 5, 6, or 7) to several weeks (1, 2, 3, 4, 5, 6, 7, or 8) lapse between respective administrations.

In certain embodiments, a course of treatment will last 1-90 days or more (this such range includes intervening days). It is contemplated that one agent may be given on any day of day 1 to day 90 (this such range includes intervening days) or any combination thereof, and another agent is given on any day of day 1 to day 90 (this such range includes intervening days) or any combination thereof. Within a single day (24-hour period), the patient may be given one or multiple administrations of the agent(s). Moreover, after a course of treatment, it is contemplated that there is a period of time at which no anti-cancer treatment is administered. This time period may last 1-7 days, and/or 1-5 weeks, and/or 1-12 months or more (this such range includes intervening days), depending on the condition of the patient, such as their prognosis, strength, health, etc. It is expected that the treatment cycles would be repeated as necessary.

Various combinations may be employed. For the example below, either (a) quinazoline-based TKI is “A” and an anti-HER2/IER3 antibody is “B” or (b) quinazoline-based TKI, either alone or in combination with an anti-HER2/HER3 antibody, is “A” and another anti-cancer therapy is “B”:

A/B/A B/A/B B/B/A A/A/B A/B/B B/A/A A/B/B/B B/A/B/B
B/B/B/A B/B/A/B A/A/B/B A/B/A/B A/B/B/A B/B/A/A
B/A/B/A B/A/A/B A/A/A/B B/A/A/A A/B/A/A A/A/B/A

Administration of any compound or therapy of the present invention to a patient will follow general protocols for the administration of such compounds, taking into account the toxicity, if any, of the agents. Therefore, in some embodiments there is a step of monitoring toxicity that is attributable to combination therapy.

1. Chemotherapy

A wide variety of chemotherapeutic agents may be used in accordance with the present invention. The term “chemotherapy” refers to the use of drugs to treat cancer. A “chemotherapeutic agent” is used to connote a compound or composition that is administered in the treatment of cancer. These agents or drugs are categorized by their mode of activity within a cell, for example, whether and at what stage they affect the cell cycle. Alternatively, an agent may be characterized based on its ability to directly cross-link DNA, to intercalate into DNA, or to induce chromosomal and mitotic aberrations by affecting nucleic acid synthesis.

Examples of chemotherapeutic agents include alkylating agents, such as thiotepa and cyclosphosphamide; alkyl sulfonates, such as busulfan, improsulfan, and piposulfan; aziridines, such as benzodopa, carboquone, meturedopa, and uredopa; ethylenimines and methylamelamines, including altretamine, triethylenemelamine, trietylenephosphoramide, triethiylenethiophosphoramide, and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards, such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, and uracil mustard; nitrosureas, such as carmustine, chlorozotocin, fotemustine, lomustine, nimustine, and ranimnustine; antibiotics, such as the enediyne antibiotics (e.g., calicheamicin, especially calicheamicin gamma 1I and calicheamicin omega I1); dynemicin, including dynemicin A; bisphosphonates, such as clodronate; an esperamicin; as well as neocarzinostatin chromophore and related chromoprotein enediyne antiobiotic chromophores, aclacinomysins, actinomycin, authrarnycin, azaserine, bleomycins, cactinomycin, carabicin, carminomycin, carzinophilin, chromomycinis, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins, such as mitomycin C, mycophenolic acid, nogalarnycin, olivomycins, peplomycin, potfiromycin, puromycin, quelamycin, rodorubicin, streptonigrin, streptozocin, tubercidin, ubenimex, zinostatin, and zorubicin; anti-metabolites, such as methotrexate and 5-fluorouracil (5-FU); folic acid analogues, such as denopterin, pteropterin, and trimetrexate; purine analogs, such as fludarabine, 6-mercaptopurine, thiamiprine, and thioguanine; pyrimidine analogs, such as ancitabine, azacitidine, 6-azauridine, carmofur, cytarabine, dideoxyuridine, doxifluridine, enocitabine, and floxuridine; androgens, such as calusterone, dromostanolone propionate, epitiostanol, mepitiostane, and testolactone; anti-adrenals, such as mitotane and trilostane; folic acid replenisher, such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids, such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSKpolysaccharide complex; razoxane; rhizoxin; sizofiran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; taxoids, e.g., paclitaxel and docetaxel gemcitabine; 6-thioguanine; mercaptopurine; platinum coordination complexes, such as cisplatin, oxaliplatin, and carboplatin; vinblastine; platinum; etoposide (VP-16); ifosfamide; mitoxantrone; vincristine; vinorelbine; novantrone; teniposide; edatrexate; daunomycin; aminopterin; xeloda; ibandronate; irinotecan (e.g., CPT-11); topoisomerase inhibitor RFS 2000; difluorometlhylornithine (DFMO); retinoids, such as retinoic acid; capecitabine; carboplatin, procarbazine, plicomycin, gemcitabien, navelbine, farnesyl-protein tansferase inhibitors, transplatinum, and pharmaceutically acceptable salts, acids, or derivatives of any of the above.

2. Radiotherapy

Other factors that cause DNA damage and have been used extensively include what are commonly known as γ-rays, X-rays, and/or the directed delivery of radioisotopes to tumor cells. Other forms of DNA damaging factors are also contemplated, such as microwaves, proton beam irradiation (U.S. Pat. Nos. 5,760,395 and 4,870,287), and UV-irradiation. It is most likely that all of these factors affect a broad range of damage on DNA, on the precursors of DNA, on the replication and repair of DNA, and on the assembly and maintenance of chromosomes. Dosage ranges for X-rays range from daily doses of 50 to 200 roentgens for prolonged periods of time (3 to 4 wk), to single doses of 2000 to 6000 roentgens. Dosage ranges for radioisotopes vary widely, and depend on the half-life of the isotope, the strength and type of radiation emitted, and the uptake by the neoplastic cells.

3. Immunotherapy

The skilled artisan will understand that additional immunotherapies may be used in combination or in conjunction with methods of the invention. In the context of cancer treatment, immunotherapeutics, generally, rely on the use of immune effector cells and molecules to target and destroy cancer cells. Rituximab (Rituxan®) is such an example. The immune effector may be, for example, an antibody specific for some marker on the surface of a tumor cell. The antibody alone may serve as an effector of therapy or it may recruit other cells to actually affect cell killing. The antibody also may be conjugated to a drug or toxin (chemotherapeutic, radionuclide, ricin A chain, cholera toxin, pertussis toxin, etc.) and serve merely as a targeting agent. Alternatively, the effector may be a lymphocyte carrying a surface molecule that interacts, either directly or indirectly, with a tumor cell target. Various effector cells include cytotoxic T cells and NK cells.

In one aspect of immunotherapy, the tumor cell must bear some marker that is amenable to targeting, i.e., is not present on the majority of other cells. Many tumor markers exist and any of these may be suitable for targeting in the context of the present invention. Common tumor markers include CD20, carcinoembryonic antigen, tyrosinase (p97), gp68, TAG-72, HMFG, Sialyl Lewis Antigen, MucA, MucB, PLAP, laminin receptor, erb B, and p155. An alternative aspect of immunotherapy is to combine anticancer effects with immune stimulatory effects. Immune stimulating molecules also exist including: cytokines, such as IL-2, IL-4, IL-12, GM-CSF, gamma-IFN, chemokines, such as MIP-1, MCP-1, IL-8, and growth factors, such as FLT3 ligand.

Examples of immunotherapies currently under investigation or in use are immune adjuvants, e.g., Mycobacterium bovis, Plasmodium falciparum, dinitrochlorobenzene, and aromatic compounds (U.S. Pat. Nos. 5,801,005 and 5,739,169; Hui and Hashimoto, Infection Immun., 66(11):5329-5336, 1998; Christodoulides et al., Microbiology, 144(Pt 11):3027-3037, 1998); cytokine therapy, e.g., interferons α, β, and γ, IL-1, GM-CSF, and TNF (Bukowski et al., Clinical Cancer Res., 4(10):2337-2347, 1998; Davidson et al., J. Immunother., 21(5):389-398, 1998; Hellstrand et al., Acta Oncologica, 37(4):347-353, 1998); gene therapy, e.g., TNF, IL-1, IL-2, and p53 (Qin et al., Proc. Natl. Acad. Sci. USA, 95(24):14411-14416, 1998; Austin-Ward and Villaseca, Revista Medica de Chile, 126(7):838-845, 1998; U.S. Pat. Nos. 5,830,880 and 5,846,945); and monoclonal antibodies, e.g., anti-CD20, anti-ganglioside GM2, and anti-p185 (Hanibuchi et al., Int. J. Cancer, 78(4):480-485, 1998; U.S. Pat. No. 5,824,311). It is contemplated that one or more anti-cancer therapies may be employed with the antibody therapies described herein.

In some embodiment, the immune therapy could be adoptive immunotherapy, which involves the transfer of autologous antigen-specific T cells generated ex vivo. The T cells used for adoptive immunotherapy can be generated either by expansion of antigen-specific T cells or redirection of T cells through genetic engineering. Isolation and transfer of tumor specific T cells has been shown to be successful in treating melanoma. Novel specificities in T cells have been successfully generated through the genetic transfer of transgenic T cell receptors or chimeric antigen receptors (CARs). CARs are synthetic receptors consisting of a targeting moiety that is associated with one or more signaling domains in a single fusion molecule. In general, the binding moiety of a CAR consists of an antigen-binding domain of a single-chain antibody (scFv), comprising the light and variable fragments of a monoclonal antibody joined by a flexible linker. Binding moieties based on receptor or ligand domains have also been used successfully. The signaling domains for first generation CARs are derived from the cytoplasmic region of the CD3zeta or the Fc receptor gamma chains. CARs have successfully allowed T cells to be redirected against antigens expressed at the surface of tumor cells from various malignancies including lymphomas and solid tumors.

In one embodiment, the present application provides for a combination therapy for the treatment of cancer wherein the combination therapy comprises adoptive T cell therapy and a checkpoint inhibitor. In one aspect, the adoptive T cell therapy comprises autologous and/or allogenic T-cells. In another aspect, the autologous and/or allogenic T-cells are targeted against tumor antigens.

Immunomodulatory agents include immune checkpoint inhibitors, agonists of co-stimulatory molecules, and antagonists of immune inhibitory molecules. The immunomodulatory agents may be drugs, such as small molecules, recombinant forms of ligand or receptors, or antibodies, such as human antibodies (e.g., International Patent Publication WO2015/016718; Pardoll, Nat Rev Cancer, 12(4): 252-264, 2012; both incorporated herein by reference). Known inhibitors of immune checkpoint proteins or analogs thereof may be used, in particular chimerized, humanized, or human forms of antibodies may be used. As the skilled person will know, alternative and/or equivalent names may be in use for certain antibodies mentioned in the present disclosure. Such alternative and/or equivalent names are interchangeable in the context of the present disclosure. For example, it is known that lambrolizumab is also known under the alternative and equivalent names MK-3475 and pembrolizumab.

Co-stimulatory molecules are ligands that interact with receptors on the surface of the immune cells, e.g., CD28, 4-1BB, OX40 (also known as CD134), ICOS, and GITR. As an example, the complete protein sequence of human OX40 has Genbank accession number NP_003318. In some embodiments, the immunomodulatory agent is an anti-OX40 antibody (e.g., a human antibody, a humanized antibody, or a chimeric antibody), an antigen binding fragment thereof, an immunoadhesin, a fusion protein, or oligopeptide. Anti-human-OX40 antibodies (or VH and/or VL domains derived therefrom) suitable for use in the present methods can be generated using methods well known in the art. Alternatively, art recognized anti-OX40 antibodies can be used. An exemplary anti-OX40 antibody is PF-04518600 (see, e.g., WO 2017/130076). ATOR-1015 is a bispecific antibody targeting CTLA4 and OX40 (see, e.g., WO 2017/182672, WO 2018/091740, WO 2018/202649, WO 2018/002339).

Another co-stimulatory molecule that can be targeted in the methods provided herein is ICOS, also known as CD278. The complete protein sequence of human ICOS has Genbank accession number NP_036224. In some embodiments, the immune checkpoint inhibitor is an anti-ICOS antibody (e.g., a human antibody, a humanized antibody, or a chimeric antibody), an antigen binding fragment thereof, an immunoadhesin, a fusion protein, or oligopeptide. Anti-human-ICOS antibodies (or VH and/or VL domains derived therefrom) suitable for use in the present methods can be generated using methods well known in the art. Alternatively, art recognized anti-ICOS antibodies can be used. Exemplary anti-ICOS antibodies include JTX-2011 (see, e.g., WO 2016/154177, WO 2018/187191) and GSK3359609 (see, e.g., WO 2016/059602).

Yet another co-stimulatory molecule that can be targeted in the methods provided herein is glucocorticoid-induced tumour necrosis factor receptor-related protein (GITR), also known as TNFRSF18 and AITR. The complete protein sequence of human GITR has Genbank accession number NP_004186. In some embodiments, the immunomodulatory agent is an anti-GITR antibody (e.g., a human antibody, a humanized antibody, or a chimeric antibody), an antigen binding fragment thereof, an immunoadhesin, a fusion protein, or oligopeptide. Anti-human-GITR antibodies (or VH and/or VL domains derived therefrom) suitable for use in the present methods can be generated using methods well known in the art. Alternatively, art recognized anti-GITR antibodies can be used. An exemplary anti-GITR antibody is TRX518 (see, e.g., WO 2006/105021).

Immune checkpoint proteins that may be targeted by immune checkpoint blockade include adenosine A2A receptor (A2AR), B7-H3 (also known as CD276), B and T lymphocyte attenuator (BTLA), CCL5, CD27, CD38, CD8A, CMKLR1, cytotoxic T-lymphocyte-associated protein 4 (CTLA-4, also known as CD152), CXCL9, CXCR5, HLA-DRB1, HLA-DQA1, HLA-E, killer-cell immunoglobulin (KIR), lymphocyte activation gene-3 (LAG-3, also known as CD223), Mer tyrosine kinase (MerTK), NKG7, programmed death 1 (PD-1), programmed death-ligand 1 (PD-L1, also known as CD274), PDCD1LG2, PSMB10, STAT1, T cell immunoreceptor with Ig and ITIM domains (TIGIT), T-cell immunoglobulin domain and mucin domain 3 (TIM-3), and V-domain Ig suppressor of T cell activation (VISTA, also known as C10orf54). In particular, immune checkpoint inhibitors targeting the PD-1 axis and/or CTLA-4 have received FDA approval broadly across diverse cancer types.

In some embodiments, a PD-1 binding antagonist is a molecule that inhibits the binding of PD-1 to its ligand binding partners. In a specific aspect, the PD-1 ligand binding partners are PD-L1 and/or PD-L2. In another embodiment, a PD-L1 binding antagonist is a molecule that inhibits the binding of PD-L1 to its binding partners. In a specific aspect, PD-L1 binding partners are PD-1 and/or B7-1. In another embodiment, a PD-L2 binding antagonist is a molecule that inhibits the binding of PD-L2 to its binding partners. In a specific aspect, a PD-L2 binding partner is PD-1. The antagonist may be an antibody, an antigen binding fragment thereof, an immunoadhesin, a fusion protein, or an oligopeptide. Exemplary antibodies are described in U.S. Pat. Nos. 8,735,553, 8,354,509, and 8,008,449, all of which are incorporated herein by reference. Other PD-1 axis antagonists for use in the methods provided herein are known in the art, such as described in U.S. Patent Application Publication Nos. 2014/0294898, 2014/022021, and 2011/0008369, all of which are incorporated herein by reference.

In some embodiments, a PD-1 binding antagonist is an anti-PD-1 antibody (e.g., a human antibody, a humanized antibody, or a chimeric antibody). In some embodiments, the anti-PD-1 antibody is selected from the group consisting of nivolumab, pembrolizumab, and CT-011. In some embodiments, the PD-1 binding antagonist is an immunoadhesin (e.g., an immunoadhesin comprising an extracellular or PD-1 binding portion of PD-L1 or PD-L2 fused to a constant region (e.g., an Fc region of an immunoglobulin sequence)). In some embodiments, the PD-1 binding antagonist is AMP-224. Nivolumab, also known as MDX-1106-04, MDX-1106, ONO-4538, BMS-936558, and OPDIVO©, is an anti-PD-1 antibody described in WO2006/121168. Pembrolizumab, also known as MK-3475, Merck 3475, lambrolizumab, KEYTRUDA©, and SCH-900475, is an anti-PD-1 antibody described in WO2009/114335. CT-011, also known as hBAT or hBAT-1, is an anti-PD-1 antibody described in WO2009/101611. AMP-224, also known as B7-DCIg, is a PD-L2-Fc fusion soluble receptor described in WO2010/027827 and WO2011/066342.

Another immune checkpoint protein that can be targeted in the methods provided herein is the cytotoxic T-lymphocyte-associated protein 4 (CTLA-4), also known as CD152. The complete cDNA sequence of human CTLA-4 has the Genbank accession number L15006. CTLA-4 is found on the surface of T cells and acts as an “off” switch when bound to CD80 or CD86 on the surface of antigen-presenting cells. CTLA-4 is similar to the T-cell co-stimulatory protein, CD28, and both molecules bind to CD80 and CD86, also called B7-1 and B7-2 respectively, on antigen-presenting cells. CTLA-4 transmits an inhibitory signal to T cells, whereas CD28 transmits a stimulatory signal. Intracellular CTLA-4 is also found in regulatory T cells and may be important to their function. T cell activation through the T cell receptor and CD28 leads to increased expression of CTLA-4, an inhibitory receptor for B7 molecules.

In some embodiments, the immune checkpoint inhibitor is an anti-CTLA-4 antibody (e.g., a human antibody, a humanized antibody, or a chimeric antibody), an antigen binding fragment thereof, an immunoadhesin, a fusion protein, or oligopeptide. Anti-human-CTLA-4 antibodies (or VH and/or VL domains derived therefrom) suitable for use in the present methods can be generated using methods well known in the art. Alternatively, art recognized anti-CTLA-4 antibodies can be used. For example, the anti-CTLA-4 antibodies disclosed in U.S. Pat. No. 8,119,129; PCT Publn. Nos. WO 01/14424, WO 98/42752, WO 00/37504 (CP675,206, also known as tremelimumab; formerly ticilimumab); U.S. Pat. No. 6,207,156; Hurwitz et al. (1998) Proc Natl Acad Sci USA, 95(17): 10067-10071; Camacho et al. (2004) J Clin Oncology, 22(145): Abstract No. 2505 (antibody CP-675206); and Mokyr et al. (1998) Cancer Res, 58:5301-5304 can be used in the methods disclosed herein. The teachings of each of the aforementioned publications are hereby incorporated by reference. Antibodies that compete with any of these art-recognized antibodies for binding to CTLA-4 also can be used. For example, a humanized CTLA-4 antibody is described in International Patent Application No. WO2001/014424, WO2000/037504, and U.S. Pat. No. 8,017,114; all incorporated herein by reference.

An exemplary anti-CTLA-4 antibody is ipilimumab (also known as 10D1, MDX-010, MDX-101, and Yervoy®) or antigen binding fragments and variants thereof (see, e.g., WO 01/14424). In other embodiments, the antibody comprises the heavy and light chain CDRs or VRs of ipilimumab. Accordingly, in one embodiment, the antibody comprises the CDR1, CDR2, and CDR3 domains of the VH region of ipilimumab, and the CDR1, CDR2, and CDR3 domains of the VL region of ipilimumab. In another embodiment, the antibody competes for binding with and/or binds to the same epitope on CTLA-4 as the above-mentioned antibodies. In another embodiment, the antibody has an at least about 90% variable region amino acid sequence identity with the above-mentioned antibodies (e.g., at least about 90%, 95%, or 99% variable region identity with ipilimumab). Other molecules for modulating CTLA-4 include CTLA-4 ligands and receptors such as described in U.S. Pat. Nos. 5,844,905, 5,885,796 and International Patent Application Nos. WO1995001994 and WO1998042752; all incorporated herein by reference, and immunoadhesins such as described in U.S. Pat. No. 8,329,867, incorporated herein by reference.

Another immune checkpoint protein that can be targeted in the methods provided herein is lymphocyte-activation gene 3 (LAG-3), also known as CD223. The complete protein sequence of human LAG-3 has the Genbank accession number NP-002277. LAG-3 is found on the surface of activated T cells, natural killer cells, B cells, and plasmacytoid dendritic cells. LAG-3 acts as an “off” switch when bound to MHC class II on the surface of antigen-presenting cells. Inhibition of LAG-3 both activates effector T cells and inhibitor regulatory T cells. In some embodiments, the immune checkpoint inhibitor is an anti-LAG-3 antibody (e.g., a human antibody, a humanized antibody, or a chimeric antibody), an antigen binding fragment thereof, an immunoadhesin, a fusion protein, or oligopeptide. Anti-human-LAG-3 antibodies (or VH and/or VL domains derived therefrom) suitable for use in the present methods can be generated using methods well known in the art. Alternatively, art recognized anti-LAG-3 antibodies can be used. An exemplary anti-LAG-3 antibody is relatlimab (also known as BMS-986016) or antigen binding fragments and variants thereof (see, e.g., WO 2015/116539). Other exemplary anti-LAG-3 antibodies include TSR-033 (see, e.g., WO 2018/201096), MK-4280, and REGN3767. MGD013 is an anti-LAG-3/PD-1 bispecific antibody described in WO 2017/019846. FS118 is an anti-LAG-3/PD-L1 bispecific antibody described in WO 2017/220569.

Another immune checkpoint protein that can be targeted in the methods provided herein is V-domain Ig suppressor of T cell activation (VISTA), also known as C10orf54. The complete protein sequence of human VISTA has the Genbank accession number NP_071436. VISTA is found on white blood cells and inhibits T cell effector function. In some embodiments, the immune checkpoint inhibitor is an anti-VISTA3 antibody (e.g., a human antibody, a humanized antibody, or a chimeric antibody), an antigen binding fragment thereof, an immunoadhesin, a fusion protein, or oligopeptide. Anti-human-VISTA antibodies (or VH and/or VL domains derived therefrom) suitable for use in the present methods can be generated using methods well known in the art. Alternatively, art recognized anti-VISTA antibodies can be used. An exemplary anti-VISTA antibody is JNJ-61610588 (also known as onvatilimab) (see, e.g., WO 2015/097536, WO 2016/207717, WO 2017/137830, WO 2017/175058). VISTA can also be inhibited with the small molecule CA-170, which selectively targets both PD-L1 and VISTA (see, e.g., WO 2015/033299, WO 2015/033301).

Another immune checkpoint protein that can be targeted in the methods provided herein is CD38. The complete protein sequence of human CD38 has Genbank accession number NP_001766. In some embodiments, the immune checkpoint inhibitor is an anti-CD38 antibody (e.g., a human antibody, a humanized antibody, or a chimeric antibody), an antigen binding fragment thereof, an immunoadhesin, a fusion protein, or oligopeptide. Anti-human-CD38 antibodies (or VH and/or VL domains derived therefrom) suitable for use in the present methods can be generated using methods well known in the art. Alternatively, art recognized anti-CD38 antibodies can be used. An exemplary anti-CD38 antibody is daratumumab (see, e.g., U.S. Pat. No. 7,829,673).

Another immune checkpoint protein that can be targeted in the methods provided herein is T cell immunoreceptor with Ig and ITIM domains (TIGIT). The complete protein sequence of human TIGIT has Genbank accession number NP_776160. In some embodiments, the immune checkpoint inhibitor is an anti-TIGIT antibody (e.g., a human antibody, a humanized antibody, or a chimeric antibody), an antigen binding fragment thereof, an immunoadhesin, a fusion protein, or oligopeptide. Anti-human-TIGIT antibodies (or VH and/or VL domains derived therefrom) suitable for use in the present methods can be generated using methods well known in the art. Alternatively, art recognized anti-TIGIT antibodies can be used. An exemplary anti-TIGIT antibody is MK-7684 (see, e.g., WO 2017/030823, WO 2016/028656).

Other immune inhibitory molecules that can be targeted for immunomodulation include STAT3 and indoleamine 2,3-dioxygenase (IDO). By way of example, the complete protein sequence of human IDO has Genbank accession number NP_002155. In some embodiments, the immunomodulatory agent is a small molecule IDO inhibitor. Exemplary small molecules include BMS-986205, epacadostat (INCB24360), and navoximod (GDC-0919).

4. Surgery

Approximately 60% of persons with cancer will undergo surgery of some type, which includes preventative, diagnostic or staging, curative, and palliative surgery. Curative surgery includes resection in which all or part of cancerous tissue is physically removed, excised, and/or destroyed and may be used in conjunction with other therapies, such as the treatment of the present invention, chemotherapy, radiotherapy, hormonal therapy, gene therapy, immunotherapy, and/or alternative therapies. Tumor resection refers to physical removal of at least part of a tumor. In addition to tumor resection, treatment by surgery includes laser surgery, cryosurgery, electrosurgery, and microscopically-controlled surgery (Mohs' surgery).

Upon excision of part or all of cancerous cells, tissue, or tumor, a cavity may be formed in the body. Treatment may be accomplished by perfusion, direct injection, or local application of the area with an additional anti-cancer therapy. Such treatment may be repeated, for example, every 1, 2, 3, 4, 5, 6, or 7 days, or every 1, 2, 3, 4, and 5 weeks or every 1, 2, 3, 4, 5, 6, 7, 8, 9, 10, 11, or 12 months. These treatments may be of varying dosages as well.

5. Other Agents

It is contemplated that other agents may be used in combination with certain aspects of the present invention to improve the therapeutic efficacy of treatment. These additional agents include agents that affect the upregulation of cell surface receptors and GAP junctions, cytostatic and differentiation agents, inhibitors of cell adhesion, agents that increase the sensitivity of the hyperproliferative cells to apoptotic inducers, or other biological agents. Increases in intercellular signaling by elevating the number of GAP junctions would increase the anti-hyperproliferative effects on the neighboring hyperproliferative cell population. In other embodiments, cytostatic or differentiation agents can be used in combination with certain aspects of the present invention to improve the anti-hyperproliferative efficacy of the treatments. Inhibitors of cell adhesion are contemplated to improve the efficacy of the present invention. Examples of cell adhesion inhibitors are focal adhesion kinase (FAKs) inhibitors and Lovastatin. It is further contemplated that other agents that increase the sensitivity of a hyperproliferative cell to apoptosis, such as the antibody c225, could be used in combination with certain aspects of the present invention to improve the treatment efficacy.

### IV. Kits

In various aspects of the invention, a kit is envisioned containing, diagnostic agents, therapeutic agents and/or delivery agents. In some embodiments, the present invention contemplates a kit for detecting an NRG1 fusion in a patient's tumor cells. In some embodiments, the present invention contemplates a kit for preparing and/or administering a therapy of the invention. The kit may comprise reagents capable of use in administering an active or effective agent(s) of the invention. Reagents of the kit may include one or more anti-cancer components of a combination therapy, as well as reagents to prepare, formulate, and/or administer the components of the invention or perform one or more steps of the inventive methods. In some embodiments, the kit may also comprise a suitable container means, which is a container that will not react with components of the kit, such as an eppendorf tube, an assay plate, a syringe, a bottle, or a tube. The container may be made from sterilizable materials such as plastic or glass. The kit may further include an instruction sheet that outlines the procedural steps of the methods, and will follow substantially the same procedures as described herein or are known to those of ordinary skill.

### V. Examples

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

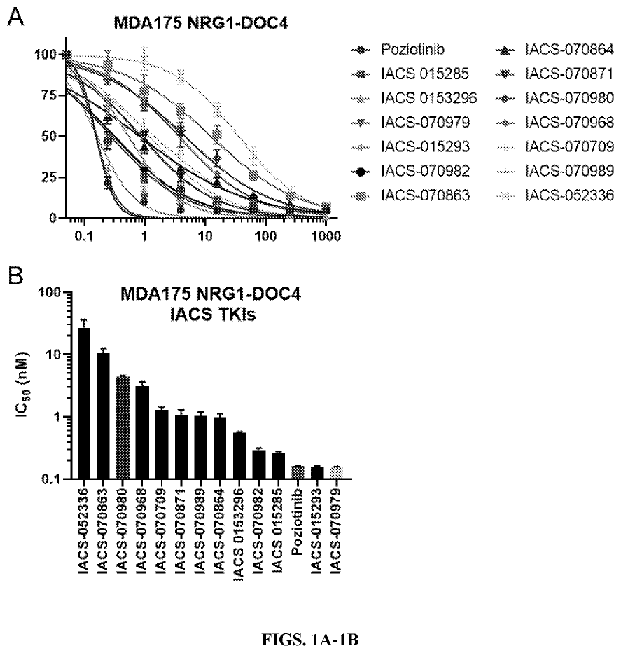

The cell viability of NRG1-DOC4 fusion breast cancer cell line, MDA175-VII, was tested with treatment of novel quinazoline-based TKIs alone and in combination with anti-HER2/3 therapies including trastuzumab, pertuzumab, and T-DM1. Cell viability was determined by the Cell Titer Glo assay. Novel quinazoline-based TKIs potently inhibited the cell viability of MDA175-VII NRG1-DOC4 fusion cells (Table 1; FIGS. 1A-1B). These data show that novel quinazoline-based TKIs potently inhibit NRG1 fusions at concentrations lower than other panHER inhibitors.

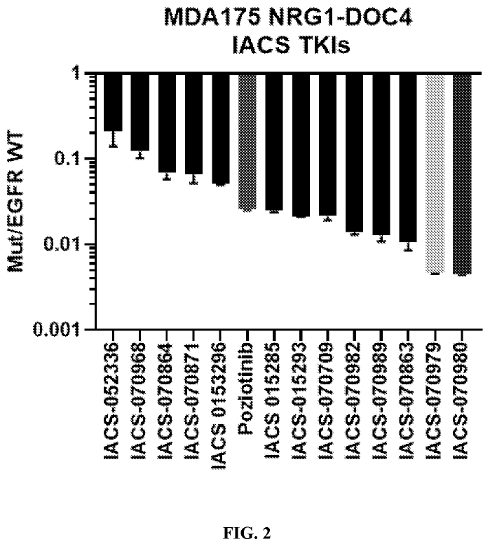

Further, because inhibition of wild-type (WT) EGFR often leads to off-target adverse events in patients, the IC₅₀ values of Ba/F3 cells expressing WT EGFR (+10 ng/μL EGF) treated with the novel quinazoline-based TKIs were determined and compared to the IC₅₀ values of cells harboring NRG1-fusions. Novel quinazoline-based TKIs were selective in inhibiting MDA175-VII NRG1 fusion cells (FIG. 2).

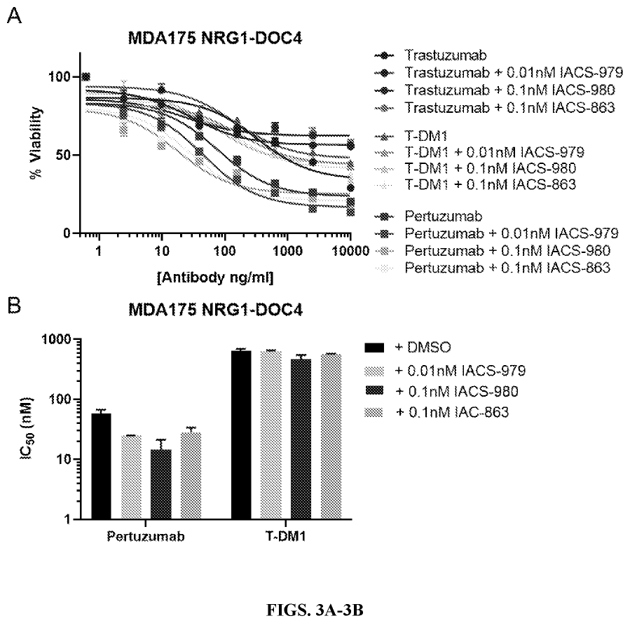

Finally, the addition of low dose quinazoline-based TKIs to anti-HER2/3 therapies led to a slight decrease in cell viability compared to anti-HER2/3 therapies alone (Table 2; FIGS. 3A-3B). These preliminary data suggest that these compounds are more potent than other panHER inhibitors tested for NRG1 fusions.

TABLE 1
Average IC₅₀ values for MDA175-VII (NRG1-DOC4
fusion) cells treated with IACS inhibitors

| Drug | Average IC₅₀, nM |
|---|---|
| IACS-015285 | 0.268 |
| IACS-015296 | 0.557 |
| IACS-070979 | 0.157 |
| IACS-015293 | 0.160 |
| IACS-070982 | 0.296 |
| IACS-070863 | 10.497 |
| IACS-070864 | 0.979 |
| IACS-070871 | 1.076 |
| IACS-070980 | 4.462 |
| IACS-070968 | 3.123 |
| IACS-070709 | 1.290 |
| IACS-070989 | 1.027 |
| IACS-052336 | 26.770 |

TABLE 2
Average IC₅₀ values for MDA175-VII (NRG1-DOC4
fusion) cells treated with anti-HER2 antibodies
with or without low dose IACS inhibitors

| Drug | Average IC₅₀, nM |
|---|---|
| Pertuzumab | 58.219 |
| Pertuzumab + 0.01 nM IACS-070979 | 24.945 |
| Pertuzumab + 0.1 nM IACS-070980 | 14.748 |
| Pertuzumab + 0.1 nM IACS-070863 | 28.298 |
| T-DM1 | 651.624 |
| T-DM1 + 0.01 nM IACS-070979 | 630.233 |
| T-DM1 + 0.1 nM IACS-070980 | 468.325 |
| T-DM1 + 0.1 nM IACS-070863 | 566.985 |

### Example 2

Ba/F3 cells generation. Ba/F3 cells stably co-expressing WT ErbB2 and WT ErbB3 or WT ErbB3 and WT ErbB4 are generated as previously described. Briefly, retroviral or lentiviral constructs are transfected into Phoenix 293T cells to produce virus which is incubated with Ba/F3 cell lines over night. Virus is removed and cells are cultured in puromycin for 10 days to select for Ba/F3 cell lines stably expressing retrovirus constructs. After selection, cells are sorted using anti-HER2, anti-HER3, and anti-HER4 antibodies (Biolegend). Cell lines are then transduced again with lentivirus containing NRG-fusion plasmids in Table 3A. Cells are then sorted by FACS for NRG1 expression. Stable cell lines are then deprived of IL-3. Resulting stable cell lines are used in downstream analyses including drug screening.

Drug screening and IC50 determination. Drug screening is performed as previously described. Briefly, cells are plated in 384-well plates (Greiner Bio-One) at 2000-3000 cells per well in technical triplicate. Seven different concentrations of quinazoline-based TKIs or DMSO vehicle are added to reach a final volume of 40 μL per well. After 72 hours, 11 μL of Cell Titer Glo (Promega) is added to each well. Plates are incubated for a minimum of 10 minutes, and bioluminescence is determined using a FLUOstar OPTIMA plate reader (BMG LABTECH). Raw bioluminescence values are normalized to DMSO control treated cells, and values are plotted in GraphPad Prism. Non-linear regressions are used to fit the normalized data with a variable slope, and IC₅₀ values are determined by GraphPad prism by interpolation of concentrations at 50% inhibition. Drug screens are performed in technical triplicate on each plate and either duplicate or triplicate biological replicates.

Overexpression models. Overexpression models are generated by lentiviral transduction of NRG1 fusions in Table 3A. Lentiviruses are generated using Lenti-X cells Lenti-X single shot kit (Takarabio). Lenti viruses are generated as described by the manufacturer. Lentiviruses are then added to the cell lines in Table 3B. After 24 hours of viral transduction, virus is removed and cells are placed into 2 μg/ml puromycin for selection. After 10 days of selection, protein and RNA are harvested from cell lines and expression of NRG1-fusions are determined by western blotting and RT-PCR, respectively. Stable cell lines with NRG1-fusion expression are used for downstream analyses including western blotting and ELISAs.

Determination inhibition of HER signaling by western blotting and ELISA in overexpressing cell lines. Parental and overexpressing (OE) cell lines are plated in 10 cm dishes and treated with quinazoline-based TKIs in increasing doses. Cells are incubated with inhibitor for a time course, and protein is harvested using lysis buffer (Cell Signaling). Expression of NRG1-fusions, phospho- and total -EGFR, HER2, HER3, and HER4 are determined by western blotting and blots are exposed using BioRad Chemidoc imager. To quantify changes in protein expression, protein from parental and OE expressing cell lines treated with quinazoline-based TKIs are loaded onto ELISAs (Cell Signaling), and ELISAs are completed by manufacturer instructions.

TABLE 1A
NRG1 fusion plasmids

| | | |
|---|---|---|
| NRG1-CD74 | NRG1-ATP1B1 | NRG1-SLC3A2 |
| NRG1-SDC4 | NRG1-VAMP1 | NRG1-CLU |
| NRG1-RBPMS | | |

TABLE 1B
Human cell line models

| Cell Line | Primary Tumor |
|---|---|
| H324 | NSCLC |
| H1819 | NSCLC |
| H2170 | NSCLC |

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
Drilon et al., Response to ERBB3-Directed Targeted Therapy in NRG1-Rearranged Cancers. Cancer Discov., 8:686-695, 2018.
Fernandez-Cuesta et al., CD74-NRG1 fusions in lung adenocarcinoma. Cancer Discov., 4:415-422, 2014.
Holbro et al., The ErbB2/ErbB3 heterodimer functions as an oncogenic unit: ErbB2 requires ErbB3 to drive breast tumor cell proliferation. Proc. Natl. Acad. Sci. USA, 100:8933-8938, 2003.
Jonna et al., Detection of NRG1 Gene Fusions in Solid Tumors. Clin. Cancer Res., 25:4966-4972, 2019.
Jung et al., VAMP2-NRG1 Fusion Gene is a Novel Oncogenic Driver of Non-Small-Cell Lung Adenocarcinoma. J. Thorac. Oncol., 10:1107-1111, 2015.
Robichaux et al., Mechanisms and clinical activity of an EGFR and HER2 exon 20-selective kinase inhibitor in non-small cell lung cancer. Nat. Med., 24:638-646, 2018.
Robichaux et al., Pan-cancer landscape and functional analysis of HER2 mutations identifies poziotinib as a clinically active inhibitor and enhancer of T-DM1 activity. Cancer Cell, 36:444-457, 2019.
Shin et al., Dual Targeting of ERBB2/ERBB3 for the Treatment of SLC3A2-NRG1-Mediated Lung Cancer. Mol. Cancer Ther., 17:2024-2033, 2018.

What is claimed is:

1. A method of treating a patient having a cancer, the method comprising (a) determining or having determined whether the patient's cancer has an NRG1 fusion; (b) selecting or having selected the patient for treatment with a quinazoline-based tyrosine kinase inhibitor (TKI) when the patient's cancer has an NRG1 fusion; and (c) administering or having administered to the selected patient a therapeutically effective amount of the quinazoline-based TKI.

2. A method of treating a patient having a cancer, the method comprising administering to the patient a therapeutically effective amount of a quinazoline-based TKI, wherein the cancer has an NRG1 fusion.

3. The method of claim 1 or 2, wherein the NRG1 fusion is an NRG1-DOC4 fusion, an NRG1-VAMP2 fusion, an NRG1-CLU fusion, an NRG1-SLC3A2 fusion, an NRG1-CD74 fusion, an NRG1-ATP1B1 fusion, or an NRG1-SDC4 fusion.

4. The method of any one of claims 1-3, wherein the quinazoline-based TKI is IACS-015285, IACS-015296, IACS-070979, IACS-015293, IACS-070982, IACS-070863, IACS-070864, IACS-070871, IACS-070980, IACS-070968, IACS-070709, IACS-070989, or IACS-052336.

5. The method of any one of claims 1-4, further comprising administering to the patient an anti-HER2/HER3 antibody.

6. The method of claim 5, wherein the anti-HER2/HER3 antibody comprises trastuzumab, pertuzumab, or T-DM1.

7. The method of any one of claims 1-6, wherein step (a) comprises (i) obtaining or having obtained a biological sample from the patient; and (ii) performing or having performed an assay on the biological sample to determine the patient's cancer has an NRG1 fusion.

8. The method of any one of claims 1-7, further comprising administering a further anti-cancer therapy to the patient.

9. The method of claim 8, wherein the further anti-cancer therapy is a surgical therapy, a chemotherapy, a radiation therapy, a cryotherapy, a hormonal therapy, a toxin therapy, an immunotherapy, or a cytokine therapy.

10. The method of any one of claims 1-9, wherein the cancer is a breast cancer, a lung cancer, a colorectal cancer, a neuroblastoma, a pancreatic cancer, a brain cancer, a stomach cancer, a skin cancer, a testicular cancer, a prostate cancer, an ovarian cancer, a liver cancer, an esophageal cancer, a cervical cancer, a head and neck cancer, a melanoma, or a glioblastoma.

11. The method of any one of claims 1-10, wherein the cancer is a breast cancer or a lung cancer.

12. The method of any one of claims 1-11, wherein the patient has previously undergone at least one round of anti-cancer therapy.

13. The method of any one of claims 1-12, further comprising reporting the presence of an NRG1 fusion in the patient's cancer.

14. The method of claim 13, wherein reporting comprises preparing a written or electronic report.

15. The method of claim 13 or 14, further comprising providing the report to the subject, a doctor, a hospital, or an insurance company.

16. A method of selecting a patient having a cancer for treatment with a quinazoline-based TKI, the method comprising (a) determining or having determined whether the patient's cancer has an NRG1 fusion; (b) selecting or having selected the patient for treatment with a quinazoline-based TKI when the patient's cancer has an NRG1 fusion.

17. The method of claim 16, wherein step (a) comprises (i) obtaining or having obtained a biological sample from the patient; and (ii) performing or having performed an assay on the biological sample to determine the patient's cancer has an NRG1 fusion.

18. The method of claim 16 or 17, further comprising (c) administering or having administered to the selected patient a therapeutically effective amount of a quinazoline-based TKI.

19. The method of any one of claims 16-18, wherein the NRG1 fusion is an NRG1-DOC4 fusion, an NRG1-VAMP2 fusion, an NRG1-CLU fusion, an NRG1-SLC3A2 fusion, an NRG1-CD74 fusion, an NRG1-ATP1B1 fusion, or an NRG1-SDC4 fusion.

20. The method of any one of claims 16-19, wherein the quinazoline-based TKI is IACS-015285, IACS-015296, IACS-070979, IACS-015293, IACS-070982, IACS-070863, IACS-070864, IACS-070871, IACS-070980, IACS-070968, IACS-070709, IACS-070989, or IACS-052336.

21. The method of claim 18 or 20, further comprising administering to the patient an anti-HER2/IER3 antibody.

22. The method of claim 21, wherein the anti-HER2/HER3 antibody comprises trastuzumab, pertuzumab, or T-DM1.

23. The method of any one of claims 18-22, further comprising administering a further anti-cancer therapy to the patient.

24. The method of claim 23, wherein the further anti-cancer therapy is a surgical therapy, a chemotherapy, a radiation therapy, a cryotherapy, a hormonal therapy, a toxin therapy, an immunotherapy, or a cytokine therapy.

25. The method of any one of claims 16-24, wherein the cancer is a breast cancer, a lung cancer, a colorectal cancer, a neuroblastoma, a pancreatic cancer, a brain cancer, a stomach cancer, a skin cancer, a testicular cancer, a prostate cancer, an ovarian cancer, a liver cancer, an esophageal cancer, a cervical cancer, a head and neck cancer, a melanoma, or a glioblastoma.

26. The method of any one of claims 16-25, wherein the cancer is a breast cancer or a lung cancer.

27. The method of any one of claims 16-26, wherein the patient has previously undergone at least one round of anti-cancer therapy.

28. The method of any one of claims 18-27, further comprising reporting the presence of an NRG1 fusion in the patient's cancer.

29. The method of claim 28, wherein reporting comprises preparing a written or electronic report.

30. The method of claim 28 or 29, further comprising providing the report to the subject, a doctor, a hospital, or an insurance company.

31. A method of treating a patient having a cancer, the method comprising (a) determining or having determined whether the patient's cancer has an NRG1 fusion; (b) selecting or having selected the patient for treatment with a quinazoline-based TKI and an anti-HER2/HER3 antibody when the patient's cancer has an NRG1 fusion; and (c) administering or having administered to the selected patient a combined therapeutically effective amount of a quinazoline-based TKI and an anti-HER2/HER3 antibody.

32. A method of treating a patient having a cancer, the method comprising administering to the patient a combined therapeutically effective amount of a quinazoline-based TKI and an anti-HER2/HER3 antibody, wherein the cancer has an NRG1 fusion.

33. The method of claim 31 or 32, wherein the NRG1 fusion is an NRG1-DOC4 fusion, an NRG1-VAMP2 fusion, an NRG1-CLU fusion, an NRG1-SLC3A2 fusion, an NRG1-CD74 fusion, an NRG1-ATP1B1 fusion, or an NRG1-SDC4 fusion.

34. The method of any one of claims 31-33, wherein the quinazoline-based TKI is IACS-015285, IACS-015296, IACS-070979, IACS-015293, IACS-070982, IACS-070863, IACS-070864, IACS-070871, IACS-070980, IACS-070968, IACS-070709, IACS-070989, or IACS-052336.

35. The method of any one of claims 31-34, wherein the anti-HER2/HER3 antibody comprises trastuzumab, pertuzumab, or T-DM1.

36. The method of any one of claims 31-35, wherein step (a) comprises (i) obtaining or having obtained a biological sample from the patient; and (ii) performing or having performed an assay on the biological sample to determine the patient's cancer has an NRG1 fusion.

37. The method of any one of claims 31-36, further comprising administering a further anti-cancer therapy to the patient.

38. The method of claim 37, wherein the further anti-cancer therapy is a surgical therapy, a chemotherapy, a radiation therapy, a cryotherapy, a hormonal therapy, a toxin therapy, an immunotherapy, or a cytokine therapy.

39. The method of any one of claims 31-38, wherein the cancer is a breast cancer, a lung cancer, a colorectal cancer, a neuroblastoma, a pancreatic cancer, a brain cancer, a stomach cancer, a skin cancer, a testicular cancer, a prostate cancer, an ovarian cancer, a liver cancer, an esophageal cancer, a cervical cancer, a head and neck cancer, a melanoma, or a glioblastoma.

40. The method of any one of claims 31-39, wherein the cancer is a breast cancer or a lung cancer.

41. The method of any one of claims 31-40, wherein the patient has previously undergone at least one round of anti-cancer therapy.

42. The method of any one of claims 31-41, further comprising reporting the presence of an NRG1 fusion in the patient's cancer.

43. The method of claim 42, wherein reporting comprises preparing a written or electronic report.

44. The method of claim 42 or 43, further comprising providing the report to the subject, a doctor, a hospital, or an insurance company.

45. A method of selecting a patient having a cancer for treatment with a quinazoline-based TKI and an anti-HER2/HER3 antibody, the method comprising (a) determining or having determined whether the patient's cancer has an NRG1 fusion; (b) selecting or having selected the patient for treatment with a quinazoline-based TKI and an anti-HER2/IER3 antibody when the patient's cancer has an NRG1 fusion.

46. The method of claim 45, wherein step (a) comprises (i) obtaining or having obtained a biological sample from the patient; and (ii) performing or having performed an assay on the biological sample to determine the patient's cancer has an NRG1 fusion.

47. The method of claim 45 or 46, further comprising (c) administering or having administered to the selected patient a combined therapeutically effective amount of a quinazoline-based TKI and an anti-HER2/HER3 antibody.

48. The method of any one of claims 45-47, wherein the NRG1 fusion is an NRG1-DOC4 fusion, an NRG1-VAMP2 fusion, an NRG1-CLU fusion, an NRG1-SLC3A2 fusion, an NRG1-CD74 fusion, an NRG1-ATP1B1 fusion, or an NRG1-SDC4 fusion.

49. The method of any one of claims 45-48, wherein the quinazoline-based TKI is IACS-015285, IACS-015296, IACS-070979, IACS-015293, IACS-070982, IACS-070863, IACS-070864, IACS-070871, IACS-070980, IACS-070968, IACS-070709, IACS-070989, or IACS-052336.

50. The method of any one of claims 45-49, wherein the anti-HER2/HER3 antibody comprises trastuzumab, pertuzumab, or T-DM1.

51. The method of any one of claims 47-50, further comprising administering a further anti-cancer therapy to the patient.

52. The method of claim 51, wherein the further anti-cancer therapy is a surgical therapy, a chemotherapy, a radiation therapy, a cryotherapy, a hormonal therapy, a toxin therapy, an immunotherapy, or a cytokine therapy.

53. The method of any one of claims 45-52, wherein the cancer is a breast cancer, a lung cancer, a colorectal cancer, a neuroblastoma, a pancreatic cancer, a brain cancer, a stomach cancer, a skin cancer, a testicular cancer, a prostate cancer, an ovarian cancer, a liver cancer, an esophageal cancer, a cervical cancer, a head and neck cancer, a melanoma, or a glioblastoma.

54. The method of any one of claims 45-53, wherein the cancer is a breast cancer or a lung cancer.

55. The method of any one of claims 45-54, wherein the patient has previously undergone at least one round of anti-cancer therapy.

56. The method of any one of claims 47-55, further comprising reporting the presence of an NRG1 fusion in the patient's cancer.

57. The method of claim 56, wherein reporting comprises preparing a written or electronic report.

58. The method of claim 56 or 57, further comprising providing the report to the subject, a doctor, a hospital, or an insurance company.
